SEQUENCE LISTING (36 sequences)

SEQ ID NO: 1 - DNA, 1494 bp, Chlamydia trachomatis
atggtgaaaagattattatgtgtgttgctatcgacatcagttttctcttcgccaatgctaggctatagtgcgtcaaagaaagattctaaggctgatatttgtcttgcagtatcctcaggagatcaagaggtttcacaagaagatctgctcaaagaagtatcccgaggattttctcgggtcgctgctaaggcaacgcctggagttgtatatatagaaaattttcctaaaacagggaaccaggctattgcttctccaggaaacaaaagaggctttcaagagaacccttttgattattttaatgacgaattttttaatcgattttttggattgccttcgcatagagagcagcagcgtccgcagcagcgtgatgctgtaagaggaactgggttcattgtttctgaagatggttatgttgttactaaccatcatgtagtcgaggatgcaggaaaaattcatgttactctccacgatggacaaaaatacacagctaagatcgtggggttagatccaaaaacagatcttgctgtgatcaaaattcaagcagagaaattaccatttttgacttttgggaattctgatcagctgcagataggtgactgggctattgctattggaaatccttttggattgcaagcaacggtcactgtcggggtcgttagtgctaaaggaagaaatcagctacatattgtagatttcgaagactttattcaaacagatgctgccattaatcctgggaattcaggcggtccattgttaaacatcaatggtcaagttatcggggttaatactgccattgtcagtggtagcgggggatatattggaatagggtttgctattcctagcttgatggctaaacgagtcattgatcaattgattagtgatgggcaggtaacaagaggctttttgggagttaccttgcaaccgatagattctgaattggctacttgttacaaattggaaaaagtgtacggagctttggtgacggatgttgttaaaggttctccagcagaaaaagcggggctgcgccaagaagatgtcattgtggcttacaatggaaaagaagtagagtctttgagtgcgttgcgtaatgccatttccctaatgatgccagggactcgtgttattttaaaaatcgttcgtgaagggaaaacaatcgagatacctgtgacggttacacagatcccaacagaggatggcgtttcagcgttgcagaagatgggagtccgtgttcagaacattactccagaaatttgtaagaaactcggattggcagcagatacccgagggattctggtagttgctgtggaggcaggctcgcctgcagcttctgcaggcgtcgctcctggacagcttatcttagcggtgaataggcagcgagtcgcttccgttgaagagttaaatcaggttttgaaaaactcgaaaggagagaatgttctccttatggtttctcaaggagatgtggtgcgattcatcgtcttgaaatcagacgagtag

SEQ ID NO: 2 - protein, 497 aa, Chlamydia trachomatis
MVKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVVSAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 3 - DNA, 1512 bp, Chlamydia trachomatis chimeric
atggtgaaaagattattatgtgtgttgctatcgacatcagttttctcttcgccaatgctaggctatagtgcgtcaaagaaagattctaaggctgatatttgtcttgcagtatcctcaggagatcaagaggtttcacaagaagatctgctcaaagaagtatcccgaggattttctcgggtcgctgctaaggcaacgcctggagttgtatatatagaaaattttcctaaaacagggaaccaggctattgcttctccaggaaacaaaagaggctttcaagagaacccttttgattattttaatgacgaattttttaatcgattttttggattgccttcgcatagagagcagcagcgtccgcagcagcgtgatgctgtaagaggaactgggttcattgtttctgaagatggttatgttgttactaaccatcatgtagtcgaggatgcaggaaaaattcatgttactctccacgatggacaaaaatacacagctaagatcgtggggttagatccaaaaacagatcttgctgtgatcaaaattcaagcagagaaattaccatttttgacttttgggaattctgatcagctgcagataggtgactgggctattgctattggaaatccttttggattgcaagcaacggtcactgtcggggtcgttagtgctaaaggaagaaatcagctacatattgtagatttcgaagactttattcaaacagatgctgccattaatcctgggaattcaggcggtccattgttaaacatcaatggtcaagttatcggggttaatactgccattgtcagtggtagcgggggatatattggaatagggtttgctattcctagcttgatggctaaacgagtcattgatcaattgattagtgatgggcaggtaacaagaggctttttgggagttaccttgcaaccgatagattctgaattggctacttgttacaaattggaaaaagtgtacggagctttggtgacggatgttgttaaaggttctccagcagaaaaagcggggctgcgccaagaagatgtcattgtggcttacaatggaaaagaagtagagtctttgagtgcgttgcgtaatgccatttccctaatgatgccagggactcgtgttattttaaaaatcgttcgtgaagggaaaacaatcgagatacctgtgacggttacacagatcccaacagaggatggcgtttcagcgttgcagaagatgggagtccgtgttcagaacattactccagaaatttgtaagaaactcggattggcagcagatacccgagggattctggtagttgctgtggaggcaggctcgcctgcagcttctgcaggcgtcgctcctggacagcttatcttagcggtgaataggcagcgagtcgcttccgttgaagagttaaatcaggttttgaaaaactcgaaaggagagaatgttctccttatggtttctcaaggagatgtggtgcgattcatcgtcttgaaatcagacgagcaccaccaccaccaccactag

SEQ ID NO: 4 - protein, 503 aa, Chlamydia trachomatis chimeric
MVKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVVSAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDHHHHHHE

SEQ ID NO: 5 - DNA, 1494 bp, Chlamydia trachomatis
atgatgaaaagattattatgtgtgttgctatcgacatcagttttctcttcgccaatgctaggctatagtgcgtcaaagaaagattctaaggctgatatttgtcttgcagtatcctcaggagatcaagaggtttcacaagaagatctgctcaaagaagtatcccgaggattttctcgggtcgctgctaaggcaacgcctggagttgtatatatagaaaattttcctaaaacagggaaccaggctattgcttctccaggaaacaaaagaggctttcaagagaacccttttgattattttaatgacgaattttttaatcgattttttggattgccttcgcatagagagcagcagcgtccgcagcagcgtgatgctgtaagaggaactgggttcattgtttctgaagatggttatgttgttactaaccatcatgtagtcgaggatgcaggaaaaattcatgttactctccacgacggacaaaaatacacagctaagatcgtggggttagatccaaaaacagatcttgctgtgatcaaaattcaagcggagaaattaccatttttgacttttgggaattctgatcagctgcagataggtgactgggctattgctattggaaatccttttggattgcaagcaacggtcactgtcggggtcattagtgctaaaggaagaaatcagctacatattgtagatttcgaagactttattcaaacagatgctgccattaatcctgggaattcaggcggtccattgttaaacatcaatggtcaagttatcggggttaatactgccattgtcagtggtagcgggggatatattggaatagggtttgctattcctagcttgatggctaaacgagtcattgatcaattgattagtgatgggcaggtaacaagaggctttttgggagttaccttgcaaccgatagattctgaattggctacttgttacaaattggaaaaagtgtacggagctttggtgacggatgttgttaaaggttctccagcagaaaaagcagggctgcgccaagaagatgtcattgtggcttacaatggaaaagaagtagagtctttgagtgcgttgcgtaatgccatttccctaatgatgccagggactcgtgttgttttaaaaatcgttcgtgaagggaaaacaatcgagatacctgtgacggttacacagatcccaacagaggatggcgtttcagcgttgcagaagatgggagtccgtgttcagaacattactccagaaatttgtaagaaactcggattggcagcagatacccgagggattctggtagttgctgtggaggcaggctcgcctgcagcttctgcaggcgtcgctcctggacagcttatcttagcggtgaataggcagcgagtcgcttccgttgaagagttaaatcaggttttgaaaaactcgaaaggagagaatgttctccttatggtttctcaaggagatgtggtgcgattcatcgtcttgaaatcagacgagtag

SEQ ID NO: 6 - protein, 497 aa, Chlamydia trachomatis
MMKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVISAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVVLKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 7 - DNA, 1494 bp, Chlamydia trachomatis
atgatgaaaagattattatgtgtgttgctatcgacatcagttttctcttcgcccatgttgggctatagtgcgccaaagaaagattccagtactggcatttgtcttgcagcatctcaaagtgatcgggaactttcccaagaagatttgctaaaagaagtgtctagaggattttccaaagtcgctgctcaggcaactccaggagttgtgtatatagaaaattttcctaaaactgggagtcaagctattgcttctcctgggaataaaaggggttttcaagagaatccctttgattatttcaatgatgagtttttcaatcgattttttggtttaccctcgcatagagagcagcctcgtccccaacagcgtgatgctgtaagaggaacaggttttattgtgtcagaagatgggtacgttgtgaccaaccatcacgtagtggaagatgcggggaaaattcatgttactttacacgatggacaaaaatacaccgcaaaaatcataggattagatcctaaaacggatctcgctgtgattaagatccaagcaaaaaatctcccttttttaacttttggaaactctgatcagcttcagataggggattggtcaatagccattggaaatcctttcggattacaagccacagtaaccgttggcgtgattagtgctaagggaagaaaccaattacatattgttgattttgaagattttattcagacggatgcagcaattaatcccgggaattcaggtggtccattattgaacattgatggacaggttattggagtgaatacagcaatcgttagcggtagcgggggatacattggaataggatttgccattcctagcttaatggctaaacgagttattgaccaactcattagcgatggacaggtgacgagaggatttttaggagtaaccttacagcctattgattcggagcttgccgcttgttacaaattagaaaaggtgtacggagccttgattacggatgttgttaagggatctcctgcagaaaaagcaggtttgcgccaggaagatgtcattgttgcttacaatgggaaagaagtggagtctttgagtgctttacgtaatgcgatttctttgatgatgccagggactcgtgttgtcttaaaagttgtgcgtgaagggaaattcattgaaatacctgtcactgttacacaaattcctgcggaggatggggtatctgctcttcaaaaaatgggagttcgggtacagaatcttactccagagatatgcaagaaactaggattagcgtctgatactcgagggatttttgtagtgtccgtagaagctggttctcctgcagcttctgcaggagtggttccaggacaacttattctggctgtaaacagacagagagtttcttctgttgaagaattgaatcaggtcttgaagaatgcaaaaggagagaatgttctccttatggtttctcaaggagaagtcattcgattcgttgttttaaagtctgatgaatag

SEQ ID NO: 8 - protein, 497 aa, Chlamydia trachomatis
MMKRLLCVLLSTSVFSSPMLGYSAPKKDSSTGICLAASQSDRELSQEDLLKEVSRGFSKVAAQATPGVVYIENFPKTGSQAIASPGNKRGFQENPFDYFNDEFFNRFFGLPSHREQPRPQQRDAVRGTGFIVSEDGYVVTNHHVVEDAGKIHVTLHDGQKYTAKIIGLDPKTDLAVIKIQAKNLPFLTFGNSDQLQIGDWSIAIGNPFGLQATVTVGVISAKGRNQLHIVDFEDFIQTDAAINPGNSGGPLLNIDGQVIGVNTAIVSGSGGYIGIGFAIPSLMAKRVIDQLISDGQVTRGFLGVTLQPIDSELAACYKLEKVYGALITDVVKGSPAEKAGLRQEDVIVAYNGKEVESLSALRNAISLMMPGTRVVLKVVREGKFIEIPVTVTQIPAEDGVSALQKMGVRVQNLTPEICKKLGLASDTRGIFVVSVEAGSPAASAGVVPGQLILAVNRQRVSSVEELNQVLKNAKGENVLLMVSQGEVIRFVVLKSDE

SEQ ID NO: 9 - DNA, 31 bp, Artificial
  synthetic primer_bind
cccggccatggtgaaaagattatgtgtgttg

SEQ ID NO: 10 - DNA, 56 bp, Artificial
  synthetic primer_bind
ccggggtcgacctagtggtggtggtggtggtgctcgtctgatttcaagacgatgaa

SEQ ID NO: 11 - DNA, 18 bp, Artificial
  synthetic primer_bind
atgatgaaaagattatta

SEQ ID NO: 12 - DNA, 16 bp, Artificial
  synthetic primer_bind
tagatccaaaaacaga

SEQ ID NO: 13 - DNA, 18 bp, Artificial
  synthetic primer_bind
aagtagagtctttgagtg

SEQ ID NO: 14 - DNA, 18 bp, Artificial
  synthetic primer_bind
atatacaactccaggcgt

SEQ ID NO: 15 - DNA, 18 bp, Artificial
  synthetic primer_bind
ggaaatggcattacgcaa

SEQ ID NO: 16 - DNA, 18 bp, Artificial
  synthetic primer_bind
ggaaatggcattacgcaa

SEQ ID NO: 17 - DNA, 18 bp, Artificial
  synthetic primer_bind
ctactcgtctgatttcaa

SEQ ID NO: 18 - protein, 171 aa, Chlamydia trachomatis
VTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 19 - protein, 61 aa, Chlamydia trachomatis
AGSPAASAGVAPGQLILAVNRQRVASVEELNQVLKNSKGENVLLMVSQGDVVRFIVLKSDE

SEQ ID NO: 20 - protein, 110 aa, Chlamydia trachomatis
VTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVE

SEQ ID NO: 21 - protein, 19 aa, Chlamydia trachomatis
MVKRLLCVLLSTSVFSSPM

SEQ ID NO: 22 - protein, 79 aa, Chlamydia trachomatis
MVKRLLCVLLSTSVFSSPMLGYSASKKDSKADICLAVSSGDQEVSQEDLLKEVSRGFSRVAAKATPGVVYIENFPKTGN

SEQ ID NO: 23 - protein, 112 aa, Chlamydia trachomatis
MVKRLLCVLLSTSVFSSPMLGYSASKKDSKADICLAVSSGDQEVSQEDLLKEVSRGFSRVAAKATPGVVYIENFPKTGNQAIASPGNKRGFQENPFDYFNDEFFNRFFGLPS

SEQ ID NO: 24 - protein, 60 aa, Chlamydia trachomatis
LGYSASKKDSKADICLAVSSGDQEVSQEDLLKEVSRGFSRVAAKATPGVVYIENFPKTGN

SEQ ID NO: 25 - protein, 93 aa, Chlamydia trachomatis
LGYSASKKDSKADICLAVSSGDQEVSQEDLLKEVSRGFSRVAAKATPGVVYIENFPKTGNQAIASPGNKRGFQENPFDYFNDEFFNRFFGLPS

SEQ ID NO: 26 - protein, 33 aa, Chlamydia trachomatis
QAIASPGNKRGFQENPFDYFNDEFFNRFFGLPS

SEQ ID NO: 27 - protein, 429 aa, Chlamydia trachomatis
VYIENFPKTGNQAIASPGNKRGFQENPFDYFNDEFFNRFFGLPSHREQQRPQQRDAVRGTGFIVSEDGYVVTNHHVVEDAGKIHVTLHDGQKYTAKIVGLDPKTDLAVIKIQAEKLPFLTFGNSDQLQIGDWAIAIGNPFGLQATVTVGVVSAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 28 - protein, 353 aa, Chlamydia trachomatis
VEDAGKIHVTLHDGQKYTAKIVGLDPKTDLAVIKIQAEKLPFLTFGNSDQLQIGDWAIAIGNPFGLQATVTVGVVSAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 29 - protein, 279 aa, Chlamydia trachomatis
VSAKGRNQLHIVDFEDFIQTDAAINPGNSGGPLLNINGQVIGVNTAIVSGSGGYIGIGFAIPSLMAKRVIDQLISDGQVTRGFLGVTLQPIDSELATCYKLEKVYGALVTDVVKGSPAEKAGLRQEDVIVAYNGKEVESLSALRNAISLMMPGTRVILKIVREGKTIEIPVTVTQIPTEDGVSALQKMGVRVQNITPEICKKLGLAADTRGILVVAVEAGSPAASAGVAPGQLILAVNRQRVASVEELNQVLKNSKGENVLLMVSQGDVVRFIVLKSDE

SEQ ID NO: 30 - protein, 127 aa, Chlamydia trachomatis fusion
AGSPAASAGVAPGQLILAVNRQRVASVEELNQVLKNSKGENVLLMVSQGDVVRFIVLKSDEGGSGGSGSPAASAGVAPGQLILAVNRQRVASVEELNQVLKNSKGENVLLMVSQGDVVRFIVLKSDE

SEQ ID NO: 31 - protein, 483 aa, Chlamydia pneumoniae
MRSWLAVLVGSGLLALPLSGQAVGKKESRVSELPQDVLLKEISGGFSKVATKATPAVVYIESFPKSQAVTHPSPGRRGPYENPFDYFNDEFFNRFFGLPSQREKPQSKEAVRGTGFLVSPDGYIVTNNHVVEDTGKIHVTLHDGQKYPATVIGLDPKTDLAVIKIKSQNLPYLSFGNSDHLKVGDWAIAIGNPFGLQATVTVGVISAKGRNQLHIADFEDFIQTDAAINPGNSGGPLLNIDGQVIGVNTAIVSGSGGYIGIGFAIPSLMANRIIDQLIRDGQVTRGFLGVTLQPIDAELAACYKLEKVYGALVTDVVKGSPADKAGLKQEDVIIAYNGKEVDSLSMFRNAVSLMNPDTRIVLKVVREGKVIEIPVTVSQAPKEDGMSALQRVGIRVQNLTPETAKKLGIAPETKGILIISVEPGSVAASSGIAPGQLILAVNRQKVSSIEDLNRTLKDSNNENILLMVSQGDVIRFIALKPEE

SEQ ID NO: 32 - DNA, 1452 bp, Chlamydia pneumoniae
ttgcgttcgtggctagctgtacttgttggttcaggtctgctagctcttcctttatcagggcaagctgtcgggaaaaaagaatctcgagtttccgagctgcctcaagacgttcttcttaaagagatctcgggagggttttctaaggtcgctaccaaggcgactcccgctgttgtgtacatagaaagtttcccaaagagccaggctgtaacacatccttctcctggacgccgtgggccttatgaaaatccttttgattattttaatgatgagtttttcaatcgtttttttggtctaccttcacagagggaaaaacctcaaagtaaagaggcggttcgaggaacaggtttcctagtatctccagatggctatattgtgactaataaccatgttgtcgaagatacaggtaagattcacgtaactcttcatgatgggcaaaagtacccagcaactgtaatcggactcgatcctaaaacagaccttgcagtcattaaaattaaatcccaaaacctcccgtatctttcttttggaaactccgaccacttaaaagtcggagattgggcaattgcaattggaaatcccttcggtcttcaagctacggtcaccgtaggtgtcatcagtgctaaaggaagaaatcaactccacattgcagattttgaagattttattcagacagatgctgcgattaatccaggcaactctggaggccctcttctaaatattgatggacaggtcatcggtgttaatactgccattgtcagtggtagtggtggctatattggaatcgggtttgcgattcctagccttatggcaaatagaatcatagatcagctgattcgtgatggtcaagttacccgaggattcttaggagtgactttacaacctatagatgcggaactcgctgcttgctacaaactcgaaaaggtttatggcgctttagtcacagatgttgttaaaggatctccagcagataaagcagggctaaaacaagaagatgtgatcattgcttataatgggaaagaagtcgattcactgagtatgttccgtaatgctgtttctttaatgaatccagatacacgtattgttctaaaggtagttcgtgaaggaaaggttatcgaaatacccgtgacagtttctcaagctccaaaagaagatggaatgtcggctttacagcgtgtgggaatccgtgtgcaaaacctaactcctgaaactgctaagaagctgggaattgctccagagactaaaggcattttgattataagtgttgaaccagggtctgtagcagcttcttcaggaattgctcctggtcagctgatccttgctgtgaatagacaaaaagtatcttcgattgaagatctgaatagaacgttaaaagattctaacaatgagaatattcttcttatggtttctcaaggagatgttattcgcttcattgccctgaaacctgaagaataa

SEQ ID NO: 33 - protein, 488 aa, Chlamydia pneumoniae
MITKQLRSWLAVLVGSSLLALPLSGQAVGKKESRVSELPQDVLLKEISGGFSKVATKATPAVVYIESFPKSQAVTHPSPGRRGPYENPFDYFNDEFFNRFFGLPSQREKPQSKEAVRGTGFLVSPDGYIVTNNHVVEDTGKIHVTLHDGQKYPATVIGLDPKTDLAVIKIKSQNLPYLSFGNSDHLKVGDWAIAIGNPFGLQATVTVGVISAKGRNQLHIADFEDFIQTDAAINPGNSGGPLLNIDGQVIGVNTAIVSGSGGYIGIGFAIPSLMANRIIDQLIRDGQVTRGFLGVTLQPIDAELAACYKLEKVYGALVTDVVKGSPADKAGLKQEDVIIAYNGKEVDSLSMFRNAVSLMNPDTRIVLKVVREGKVIEIPVTVSQAPKEDGMSALQRVGIRVQNLTPETAKKLGIAPETKGILIISVEPGSVAASSGIAPGQLILAVNRQKVSSIEDLNRTLKDSNNENILLMVSQGDVIRFIALKPEE

SEQ ID NO: 34 - DNA, 1467 bp, Chlamydia pneumoniae
atgataactaagcaattgcgttcgtggctagctgtacttgttggttcaagtctgctagctcttcctttatcagggcaagctgtcgggaaaaaagaatctcgagtttccgagctgcctcaagacgttcttcttaaagagatctcgggagggttttctaaggtcgctaccaaggcgactcccgctgttgtgtacatagaaagtttcccaaagagccaggctgtaacacatccttctcctggacgccgtgggccttatgaaaatccttttgattattttaatgatgagtttttcaatcgtttttttggtctaccttcacagagggaaaaacctcaaagtaaagaggcggttcgaggaacaggtttcctagtatctccagatggctatattgtgactaataaccatgttgtcgaagatacaggtaagattcacgtaactcttcatgatgggcaaaagtacccagcaactgtaatcggactcgatcctaaaacagaccttgcagtcattaaaattaaatcccaaaacctcccgtatctttcttttggaaactccgaccacttaaaagtcggagattgggcaattgcaattggaaatcccttcggtcttcaagctacggtcaccgtaggtgtcatcagtgctaaaggaagaaatcaactccacattgcagattttgaagattttattcagacagatgctgcgattaatccaggcaactctggaggccctcttctaaatattgatggacaggtcatcggtgttaatactgccattgtcagtggtagtggtggctatattggaatcgggtttgcgattcctagccttatggcaaatagaatcatagatcagctgattcgtgatggtcaagttacccgaggattcttaggagtgactttacaacctatagatgcggaactcgctgcttgctacaaactcgaaaaggtttatggcgctttagtcacagatgttgttaaaggatctccagcagataaagcagggctaaaacaagaagatgtgatcattgcttataatgggaaagaagtcgattcactgagtatgttccgtaatgctgtttctttaatgaatccagatacacgtattgttctaaaggtagttcgtgaaggaaaggttatcgaaatacccgtgacagtttctcaagctccaaaagaagatggaatgtcggctttacagcgtgtgggaatccgtgtgcaaaacctaactcctgaaactgctaagaagctgggaattgctccagagactaaaggcattttgattataagtgttgaaccagggtctgtagcagcttcttcaggaattgctcctggtcagctgatccttgctgtgaatagacaaaaagtatcttcgattgaagatctgaatagaacgttaaaagattctaacaatgagaatattcttcttatggtttctcaaggagatgttattcgcttcattgccctgaaacctgaagaataa

SEQ ID NO: 35 - protein, 488 aa, Chlamydia pneumoniae
  MISC_FEATURE (17)..(17): Xaa can be any amino acid
MITKQLRSWLAVLVGSXLLALPLSGQAVGKKESRVSELPQDVLLKEISGGFSKVATKATPAVVYIESFPKSQAVTHPSPGRRGPYENPFDYFNDEFFNRFFGLPSQREKPQSKEAVRGTGFLVSPDGYIVTNNHVVEDTGKIHVTLHDGQKYPATVIGLDPKTDLAVIKIKSQNLPYLSFGNSDHLKVGDWAIAIGNPFGLQATVTVGVISAKGRNQLHIADFEDFIQTDAAINPGNSGGPLLNIDGQVIGVNTAIVSGSGGYIGIGFAIPSLMANRIIDQLIRDGQVTRGFLGVTLQPIDAELAACYKLEKVYGALVTDVVKGSPADKAGLKQEDVIIAYNGKEVDSLSMFRNAVSLMNPDTRIVLKVVREGKVIEIPVTVSQAPKEDGMSALQRVGIRVQNLTPETAKKLGIAPETKGILIISVEPGSVAASSGIAPGQLILAVNRQKVSSIEDLNRTLKDSNNENILLMVSQGDVIRFIALKPEE

SEQ ID NO: 36 - DNA, 1467 bp, Chlamydia pneumoniae
atgataactaagcaattgcgttcgtggctagctgtacttgttggttcargtctgctagctcttcctttatcagggcaagctgtcgggaaaaaagaatctcgagtttccgagctgcctcaagacgttcttcttaaagagatctcgggagggttttctaaggtcgctaccaaggcgactcccgctgttgtgtacatagaaagtttcccaaagagccaggctgtaacacatccttctcctggacgccgtgggccttatgaaaatccttttgattattttaatgatgagtttttcaatcgtttttttggtctaccttcacagagggaaaaacctcaaagtaaagaggcggttcgaggaacaggtttcctagtatctccagatggctatattgtgactaataaccatgttgtcgaagatacaggtaagattcacgtaactcttcatgatgggcaaaagtacccagcaactgtaatcggactcgatcctaaaacagaccttgcagtcattaaaattaaatcccaaaacctcccgtatctttcttttggaaactccgaccacttaaaagtcggagattgggcaattgcaattggaaatcccttcggtcttcaagctacggtcaccgtaggtgtcatcagtgctaaaggaagaaatcaactccacattgcagattttgaagattttattcagacagatgctgcgattaatccaggcaactctggaggccctcttctaaatattgatggacaggtcatcggtgttaatactgccattgtcagtggtagtggtggctatattggaatcgggtttgcgattcctagccttatggcaaatagaatcatagatcagctgattcgtgatggtcaagttacccgaggattcttaggagtgactttacaacctatagatgcggaactcgctgcttgctacaaactcgaaaaggtttatggcgctttagtcacagatgttgttaaaggatctccagcagataaagcagggctaaaacaagaagatgtgatcattgcttataatgggaaagaagtcgattcactgagtatgttccgtaatgctgtttctttaatgaatccagatacacgtattgttctaaaggtagttcgtgaaggaaaggttatcgaaatacccgtgacagtttctcaagctccaaaagaagatggaatgtcggctttacagcgtgtgggaatccgtgtgcaaaacctaactcctgaaactgctaagaagctgggaattgctccagagactaaaggcattttgattataagtgttgaaccagggtctgtagcagcttcttcaggaattgctcctggtcagctgatccttgctgtgaatagacaaaaagtatcttcgattgaagatctgaatagaacgttaaaagattctaacaatgagaatattcttcttatggtttctcaaggagatgttattcgcttcattgccctgaaacctgaagaataa

(19) United States
(12) Patent Application Publication
(10) Pub. No.: US 2011/0027321 A1
(43) Pub. Date: Feb. 3, 2011

(54) Chlamydia Vaccine Comprising HtrA Polypeptides

(75) Inventors: Hang Lu, Gaithersbyrg, MD (US); Gary S. Nabors, Damascus, MD (US); W. James Jackson, Marriottsville, MD (US); Xuan Z. Ding, Rockville, MD (US); Steven F. Roberts, Bethesda, MD (US)

Correspondence Address: Sterne, Kessler, Goldstein & Fox, P.L.L.C., 1100 New York Avenue, NW, Washington, DC, 20005, US

(73) Assignee: EMERGENT PRODUCT DEVELOPMENT GAITHERSBURG INC., Gaithersburg, MD (US)

(21) Appl. No.: 12/601,431

(22) PCT Filed: May 23, 2008
(86) PCT No.: PCT/US2008/006656

§ 371 Date: Oct. 20, 2010

Related U.S. Application Data
(60) Provisional application No. 60/940,327, filed on May 25, 2007.

(51) Int. Cl. A61K 39/118; C12N 15/31; C12N 15/63; C12N 1/00; A61K 31/7052; A61K 38/16; C12P 21/00; C12Q 1/68; G01N 33/571; A61P 37/00; A61P 31/04

(52) U.S. Cl. 424/263.1; 536/23.7; 435/320.1; 435/243; 514/44 R; 514/21.2; 435/69.1; 435/6; 435/7.36

(57) ABSTRACT

The present invention provides vaccine compositions useful in prevention and treatment of Chlamydia sp. (e.g., C. trachomatis or C. pneumoniae) infection. Provided are polypeptide vaccine antigens comprising Chlamydia HtrA-derived sequences, including epitopic fragments, analogs, derivatives, and variants. Also provided are a method for inducing an immune response to a subject against Chlamydia infection, a method for preventing Chlamydia infection, or a method for treating a disease or symptom caused by or resulting from infection with Chlamydia, for instance, C. trachomatis or C. pneumoniae. In one embodiment, the present invention is drawn to C. trachomatis HtrA, which induces a cellular immune response and imparts partial protective immunity in vivo.

### BACKGROUND OF THE INVENTION

### Field of Invention

This invention relates to a vaccine useful against infection by Chlamydia sp. (e.g., C. trachomatis and C. pneumoniae), a process for making the vaccine, and a method for immunizing a human or animal against Chlamydia infection.

Bacteria of the family Chlamydiaceae infect a variety of host species and are associated with a wide range of different disease pathologies, including genital, ocular and neonatal infection. C. trachomatis is the world's most common cause of sexually transmitted disease. The World Health Organization estimates that at least 90 million people are infected each year. In the United States, genital infection with C. trachomatis is the single most frequently reported infectious disease, with an estimated 4 million cases per year. Approximately 10% of women suffering C. trachomatis genital infection eventually become infertile. Children born to C. trachomatis-infected mothers are at high risk of ocular and respiratory infection. C. trachomatis is also a leading cause of ocular infection in tropical and sub-tropical nations, causing blindness in an estimated 6 million people per year. (Schachter, Julius, Chapter 6, “Infection and Disease Epidemiology,” pp 139-169, in Chlamydia: Intracellular Biology, Pathogenesis and Immunity, Stephens, Richard S., ed., ASM Press, Washington, D.C., 1999; Hogan, et al., Infect Immun., 72(4):1843-555 (2004); Peipert, N Engl J Med, 349:2424-30 (2003)).

Chlamydiaceae possess a unique developmental cycle which allows them to evade host immune responses: Chlamydiaceae exist inside a eukaryotic host cell as a non-infectious reticulate body (RB), or, alternately, outside a host cell as an infectious elementary body (EB). Chlamydia reproduces inside the host cell as RBs then develop into EBs, which exit the host in order to spread to neighboring cells. The critical step in establishment of Chlamydia infection is adhesion of an infectious EB to a target cell followed by receptor-mediated endocytosis, or other mechanism of high-affinity absorption, termed “parasite specific endocytosis.” Chlamydia cell surface antigens are widely believed to direct both “parasite specific endocytosis” and also host immune responses.

In general, antibiotic treatment of C. trachomatis genital infection is effective only during early stages of infection, which are typically asymptomatic. Most victims don't realize they're infected until later stages, when antibiotics are no longer particularly useful. Accordingly, efforts have been undertaken worldwide to identify whole organism or sub-unit vaccines that are protective against C. trachomatis infection.

No effective vaccine against C. trachomatis infection has yet been developed. A significant impediment to vaccine development has been that the hosts' own immune responses contribute to pathogenesis of infection. Pathologies arise from persistent inflammation rather than from intrinsic destructiveness of the Chlamydia organism. Some anti-Chlamydia antibodies actively exacerbate infection, promoting its spread by enhancing “parasite specific endocytosis.” Ideally, an effective vaccine will not include “sensitizing antigens” which promote an ineffective or even destructive humoral immune response without contributing to protective immunity. (Bailey, et al., Epidemiol. Infec., 111(2):315-24, 1993; Stephens, Richard S., Introduction, pp xi-xxii, and Schachter, Julius, Chapter 6, “Infection and Disease Epidemiology,” pp 139-169, and Rank, Roger, Chapter 9, “Models of Immunity,” pp 239-295, in Chlamydia: Intracellular Biology, Pathogenesis and Immunity, Stephens, Richard S., ed., ASM Press, Washington, D.C., 1999).

The molecular mechanisms of immunity to C. trachomatis remain elusive. In humans, immunity provided by prior infection is generally incomplete, short-lived and specific to one of 18 different serovars of C. trachomatis. Because Chlamydia live and reproduce inside a host cell as RBs, host immune responses mediated by blood-borne antibodies cannot clear infections. Some antibodies are “neutralizing” in vitro, in that they prevent the spread of infectious EBs in cell culture experiments. However, standing alone, these “neutralizing” antibodies are not sufficient to impart protective immunity. For example, most people suffering from chronic C. trachomatis genital infection have high levels of anti-Chlamydia IgG which are “neutralizing” in vitro. (Stephens, Richard S., Introduction, pp xi-xxii, and Schachter, Julius, Chapter 6, “Infection and Disease Epidemiology,” pp 139-169, and Brunham, Robert, Chapter 8, “Human Immunity to Chlamydiae,” pp 211-238, in Chlamydia: Intracellular Biology, Pathogenesis and Immunity, Stephens, Richard S., ed., ASM Press, Washington, D.C., 1999).

Furthermore, “neutralizing” antibodies are completely unnecessary for protective immunity in some animal models. Protective immunity in vivo against C. trachomatis appears primarily due to cellular immune responses. While some anti-Chlamydia antibodies actively exacerbate infection, antibodies generated against known protective antigens contribute to protective immunity by contributing to immune memory and optimum cellular immune response. Ideally, an effective vaccine antigen will promote a cellular immune response as well as a humoral immune response that contributes to cross-strain or genus-specific protective immunity. Id.

High levels of antibodies that were “neutralizing” in vitro were produced in response to immunization with one promising, genetically engineered antigen. This recombinant, chimeric polypeptide was derived from known epitopes of the immunodominant C. trachomatis major outer membrane protein (MOMP). A known T-helper epitope (directing a cellular immune response) was combined with known B-cell epitopes (directing a humoral immune response). Surprisingly, this highly immunogenic, “neutralizing” antigen failed to impart protective immunity in vivo. (Su, et al., Vaccine, 13(11):1023-1032, 1995). A similar situation was observed with a cysteine-rich, 61 kDa C. trachomatis outer membrane protein. The cysteine-rich 61 kD protein was highly immunogenic, but failed to impart protective immunity in vivo. (Bowie, et al., Chlamydial infections: proceedings of the Seventh International Symposium on Human Chlamydial Infections, British Columbia, Canada, 24-19 Jun. 1990, pp 265-268, Cambridge University Press).

Because complete genome sequences are known from two strains of C. trachomatis (Stephens, et al., Science, 282(5389):754-9, 1998; Read, et al., Nucleic Acids Res., 28(6):1397-406, 2000), polypeptides that might be useful as vaccine antigens can be preliminarily selected using computerized, purely bioinformatic approaches. For example, Grandi et al., WO 03/049762 used sequence comparisons with known vaccine antigens from other species to identify 130 “candidate” C. trachomatis antigens. Each of these “candidates” was recombinantly expressed and screened for immunogenicity. Anti-sera raised against some of the most immunogenic prospective antigens were also tested for “neutralization” of C. trachomatis infection in vitro.

One prospective C. trachomatis vaccine antigen tested by Grandi et al. was the HtrA (high temperature requirement) serine protease. HtrA is an important heat-shock inducible protein. It acts both as a molecular chaperone under normal conditions, facilitating proper folding of cellular proteins, and also as protease under conditions of heat stress, digesting improperly folded proteins. HtrAs generally have a broad substrate specificity, recognizing cleavage sites from solvent-protected regions of proteins that would not normally be exposed at the protein surface. In vitro testing, carried out by Grandi et al. identified HtrA as an antigen that stimulates high levels of antibodies that are not “neutralizing” in vitro. (Grandi, et al., Genomics, Proteomics and Vaccines, Part 11 Searching the Chlamydia Genomes for New Vaccine Candidates, pp 245-266, John Wiley & Sons, Ltd., West Sussex, England, 2004).

### SUMMARY OF THE INVENTION

The present invention includes isolated polynucleotides which encode Chlamydia HtrA polypeptides, including, for instance, isolated polynucleotides which encode C. trachomatis and C. pneumoniae HtrA polypeptides. In certain embodiments, the present invention is directed to an isolated polynucleotide which encodes a polypeptide comprising an amino acid sequence at least 75% identical to a reference amino acid sequence selected from a group consisting of amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ID NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 30; and a combination of at least two of any of said polypeptide fragments or variants or derivatives thereof, provided that the amino acid sequence is not SEQ ID NO: 6, or SEQ ID NO: 8; where the polypeptide, when administered to a subject in need thereof in a sufficient amount, induces an immune response against Chlamydia sp. The present invention also includes an isolated polynucleotide comprising a nucleic acid which encodes amino acids 21-497 of SEQ ID NO: 2.

Also included is an isolated polynucleotide comprising a nucleic acid which encodes an amino acid sequence at least 75% identical to a reference amino acid sequence selected from the group consisting of: amino acids a-483 of SEQ ID NO: 2; amino acids 22-b of SEQ ID NO: 2; amino acids a-b of SEQ ID NO: 2; amino acids c-379 of SEQ ID NO: 2; amino acids 300-d of SEQ ID NO: 2; amino acids c-d of SEQ ID NO: 2; amino acids e-483 of SEQ ID NO: 2; amino acids 428-b of SEQ ID NO: 2; amino acids e-b of SEQ ID NO: 2; amino acids c-483 of SEQ ID NO: 2; amino acids 300-b of SEQ ID NO: 2; amino acids c-b of SEQ ID NO: 2; amino acids f-288 of SEQ ID NO: 2; amino acids 128-g of SEQ ID NO: 2; amino acids f-g of SEQ ID NO: 2; amino acids f-379 of SEQ ID NO: 2; amino acids 128-d of SEQ ID NO: 2; amino acids f-d of SEQ ID NO: 2; amino acids f-483 of SEQ ID NO: 2; amino acids 128-b of SEQ ID NO: 2; amino acids f-b of SEQ ID NO: 2; amino acids a-379 of SEQ ID NO: 2; amino acids 22-d of SEQ ID NO: 2; amino acids a-d of SEQ ID NO: 2; amino acids a-288 of SEQ ID NO: 2; amino acids 22-g of SEQ ID NO: 2; and amino acids a-g of SEQ ID NO: 2; wherein a is any integer between 17 and 22, b is any integer between 483-497, c is any integer between 290-300, d is any integer between 379-389, e is any integer between 394-428, f is any integer between 95-128, and g is any integer between 288 and 297. In another embodiment, the present invention is directed to the polynucleotide of the present invention, where the polynucleotide does not include a polynucleotide sequence encoding an amino acid sequence selected from the group consisting of: amino acids f-288 of SEQ ID NO: 2; amino acids 128-g of SEQ ID NO: 2; amino acids f-g of SEQ ID NO: 2; amino acids c-379 of SEQ ID NO: 2; amino acids 300-d of SEQ ID NO: 2; and amino acids c-d of SEQ ID NO: 2, where c is any integer between 290-300, d is any integer between 379-389, f is any integer between 95-128, and g is any integer between 288-297, provided that the amino acid sequence is not SEQ ID NO: 6, or SEQ ID NO: 8, and wherein the polypeptides encoded by the polynucleotides induce an immune response against Chlamydia sp. when administered to a subject in need thereof in a sufficient amount.

Also provided is an isolated polynucleotide as described above, which encodes a polypeptide with no or reduced catalytic activity. For instance, the polynucleotide may encode a polypeptide having one or more amino acid substitutions at a residue selected from the group consisting of residue 105 of SEQ ID NO: 2, residue 137 of SEQ ID NO: 2, residue 143 of SEQ ID NO: 2, residue 155 of SEQ ID NO: 2, residue 247 of SEQ ID NO: 2 and a combination of two or more residues.

In certain embodiments, the reference amino acid sequence is selected from the group consisting of: amino acids 22-483 of SEQ ID NO: 2; amino acids 17-483 of SEQ ID NO: 2; amino acids 22-497 of SEQ ID NO: 2; amino acids 17-497 of SEQ ID NO: 2; amino acids 290-381 of SEQ ID NO: 2; amino acids 290-379 of SEQ ID NO: 2; amino acids 300-382 of SEQ ID NO: 2; amino acids 291-381 of SEQ ID NO: 2; amino acids 300-389 of SEQ ID NO: 2; amino acids 394-485 of SEQ ID NO: 2; amino acids 428-483 of SEQ ID NO: 2; amino acids 404-486 of SEQ ID NO: 2; amino acids 407-485 of SEQ ID NO: 2; amino acids 404-493 of SEQ ID NO: 2; amino acids 290-485 of SEQ ID NO: 2; amino acids 290-483 of SEQ ID NO: 2; amino acids 300-486 of SEQ ID NO: 2; amino acids 291-485 of SEQ ID NO: 2; amino acids 300-493 of SEQ ID NO: 2; amino acids 128-381 of SEQ ID NO: 2; amino acids 128-379 of SEQ ID NO: 2; amino acids 110-382 of SEQ ID NO: 2; amino acids 95-389 of SEQ ID NO: 2; amino acids 128-485 of SEQ ID NO: 2; amino acids 128-483 of SEQ ID NO: 2; amino acids 110-486 of SEQ ID NO: 2; and amino acids 95-493 of SEQ ID NO: 2.

The present invention is also directed to a polynucleotide as described above further comprising a heterologous nucleic acid. The polynucleotide in some embodiments encodes a polypeptide which has reduced or decreased catalytic activity.

In another embodiment, the coding region encoding the HtrA polypeptide is codon-optimized.

Also included is a vector comprising the polynucleotide or a host cell comprising the vector. In certain embodiments, the invention includes a method of producing a polypeptide, comprising culturing the host cell and recovering the polypeptide. For instance, the present invention includes the use of a baculovirus expression system to produce a Chlamydia HtrA polypeptide in an insect host cell.

The present invention further includes an isolated polypeptide encoded by a polynucleotide described above, or a composition comprising such a polypeptide. The composition can further comprise an adjuvant. For instance, the present invention includes a C. trachomatis or C. pneumoniae HtrA polypeptide or polypeptide fragment which exhibits reduced or decreased protease activity. In one embodiment, the HtrA polypeptide or polypeptide fragment of the invention exhibits no protease activity.

The invention includes a Chlamydia HtrA polypeptide or polypeptide fragment comprising one or more modified protease cleavage sites. In one embodiment, the HtrA polypeptide or polypeptide of the invention is protease resistant.

In other embodiment, the invention is directed to a method for determining the presence of nucleic acids of Chlamydia sp. in a test sample, comprising the steps of: a) contacting the test sample with the polynucleotide or complement sequence thereof to produce duplexes; and b) determining the production of duplexes or a method of detecting Chlamydia in a test sample comprising the steps of: a) contacting the test sample with the antibody against the polypeptide to form antigen: antibody immunocomplexes, and further, b) detecting the presence of or measuring the amount of said immunocomplexes formed during step a). Further included is a method of detecting antibodies against Chlamydia in a test sample comprising the steps of: contacting the test sample with the polypeptide to form Chlamydia antigen: antibody immunocomplexes, and further, b) detecting the presence of or measuring the amount of said immunocomplexes formed during step a).

The present invention further includes a method of inducing an immune response against Chlamydia in a subject comprising administering to a subject in need thereof an effective amount of a polynucleotide, a vector, a host cell, a polypeptide, or a composition of the present invention. The immune response is an antibody response and/or a cellular immune response. The immune response can be a mucosal immune response.

In some embodiments, the invention is directed to a method to treat or prevent a Chlamydia infection in a subject comprising administering to a subject in need thereof a polynucleotide, a vector, a host cell, a polypeptide, or a composition of the present invention; or a method to attenuate or ameliorate a symptom caused by a Chlamydia infection in a subject comprising administering to a subject in need thereof a polynucleotide, a vector, a host cell, a polypeptide, or a composition of the present invention. For instance, the invention includes, but is not limited to methods of preventing a C. trachomatis or C. pneumoniae infection or disease associated with a C. trachomatis (e.g., prostatitis, urethritis, epididymitis, cervicitis, pelvic inflammatory disease, pelvic pain, newborn eye infection, newborn lung infection, infertility, proctitis, reactive arthritis and trachoma) or C. pneumoniae infection (e.g., pneumonia, acute respiratory disease, atherosclerosis, coronary artery disease, myocardial infarction, carotid artery disease, cerebrovascular disease, coronary heart disease, carotid artery stenosis, aortic aneurysm, claudication and stroke).

The invention includes a method of treating or preventing a Chlamydia infection in a subject comprising administering to a subject an attenuated, non-Chlamydia organism expressing the HtrA protein of the present invention. For instance, the invention includes a method of treating or preventing a Chlamydia infection in a subject comprising administering an attenuated gram-negative pathogen such as S. typhi or an attenuated virus such as Modified Vaccinia Virus Ankara (MVA) which expresses a Chlamydia HtrA protein or fragment.

In some embodiments, the invention is directed to a method of providing passive immunity comprising administering the antibody reactive with the Chlamydia organism to an animal in need thereof.

The sequence identifiers used herein are as follows:
SEQ ID NO: 1: C. trachomatis (L₂) HtrA nucleotide sequence.
SEQ ID NO: 2: C. trachomatis HtrA (L₂) polypeptide sequence.
SEQ ID NO: 3: C. trachomatis HtrA (L₂) chimeric nucleotide sequence.
SEQ ID NO: 4: C. trachomatis HtrA (L₂) chimeric polypeptide sequence.
SEQ ID NO: 5: C. trachomatis (D) HtrA nucleotide sequence.
SEQ ID NO: 6: C. trachomatis HtrA (D) polypeptide sequence.
SEQ ID NO: 7: C. trachomatis (MoPN) HtrA nucleotide sequence.
SEQ ID NO: 8: C. trachomatis HtrA (MoPN) polypeptide sequence.
SEQ ID NO: 9-17: primers.
SEQ ID NO: 18-29: Epitopic fragments of full length C. trachomatis HtrA polypeptide.
SEQ ID NO: 30: C. trachomatis HtrA fusion polypeptide
SEQ ID NO: 31: C. pneumoniae (TW-183) HtrA polypeptide sequence
SEQ ID NO: 32: C. pneumoniae (TW-183) HtrA polynucleotide sequence
SEQ ID NO: 33: C. pneumoniae (J138 and CWL029) HtrA polypeptide sequence
SEQ ID NO: 34: C. pneumoniae (J138 and CWL029) HtrA polynucleotide sequence
SEQ ID NO: 35: C. pneumoniae (AR39) HtrA polypeptide sequence
SEQ ID NO: 36: C. pneumoniae (AR39) HtrA polynucleotide sequence

### BRIEF DESCRIPTION OF THE DRAWINGS

FIG. 1—a conserved domain map of C. trachomatis HtrA. The structural domains are a serine protease domain and two PDZ protein-protein interaction domains.

FIG. 2—Nucleotide sequence alignment of nucleotide sequences encoding full-length HtrA polypeptide from C. trachomatis serotypes L₂, D, and MoPn (SEQ ID NOs: 1, 5, and 7).

FIG. 3—Amino acid sequence alignment of full-length HtrA polypeptide from C. trachomatis serotypes L₂, D, and MoPn (SEQ ID NOs: 2, 6, and 8).

FIG. 4(A)—Recombinant Chlamydia trachomatis HtrA protein is recognized by anti-pentaHis antibody in Western blot analysis. Lane 1: molecular weight standard; Lane 2: 4.2 μg Chlamydia trachomatis HtrA protein; and Lane 3: 5.0 mg Chlamydia trachomatis HtrA protein.

FIG. 4(B)—Recombinant Chlamydia trachomatis HtrA protein exhibits Protease Activity in Zymogram Gels. Lane 1: molecular weight standards and Lane 2: 5.7 μg Chlamydia trachomatis HtrA protein.

FIG. 4(C)—Purification of recombinant Chlamydia trachomatis HtrA protein from E. coli (DE3) (pET28-CtHtrA/L₂) Lane 1: molecular weight standard; Lane 2: 4.2 μg Chlamydia trachomatis HtrA protein; and Lane 3: 5.0 μg Chlamydia trachomatis HtrA protein.

FIG. 4(D)—Recombinant Chlamydia trachomatis HtrA protein is recognized by anti-Chlamydia pneumoniae HtrA antibody in western blot analysis. Lane 1: molecular weight standards; Lane 2: 4.2 μg Chlamydia trachomatis HtrA protein; and Lane 3: 5.0 μg Chlamydia trachomatis HtrA protein.

FIG. 5—Spleen cell proliferative response to recombinant C. trachomatis HtrA. Panel A shows stimulation index for each group of cultures restimulated with HtrA. Panel B shows stimulation index for each group of cultures challenged with ConA.

FIG. 6—Serum anti-HtrA IgG titers after immunization with either 10 or 50 μg recombinant C. trachomatis HtrA or PBS.

FIG. 7—Effect of prior immunization with recombinant C. trachomatis HtrA serovar L₂ on genital recovery of animals subject to vaginal challenge with serovar E.

FIG. 8—Effect of prior immunization with recombinant C. trachomatis HtrA serovar L₂ on total vaginal exposure to C. trachomatis serovar E during 14 days post-infection of animals subject to vaginal challenge with serovar E.

### DETAILED DESCRIPTION OF THE INVENTION

The present invention is directed to polypeptides and polynucleotides derived from the genus Chlamydia, composition comprising the polypeptides and polynucleotides, and methods of administering the composition to prevent or treat Chlamydia infection. Examples of suitable Chlamydia species include, but are not limited to, Chlamydia trachomatis, Chlamydia psittaci, Chlamydia pecorum, and Chlamydia pneumoniae. For instance, the present invention discloses that, while not identified as a “neutralizing” antigen in vitro, isolated recombinant HtrA polypeptides or polynucleotides encoded by the polypeptides from C. trachomatis induce a cellular immune response and impart protective immunity in vivo.

Methods of making and using the present invention include all conventional techniques of molecular biology, microbiology, immunology, and vaccination. Such techniques are set forth in the literature including but not limited to e.g. Sambrook Molecular Cloning; A Laboratory Manual, Second Edition (1989) and Third Edition (2001); Genetic Engineering: Principles and Methods, Volumes 1-25 (J. K. Setlow ed, 1988); DNA Cloning, Volumes I and II (D. N Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification Principles and Practice, Second Edition (Springer-Verlag, N.Y.), and Handbook of Experimental Immunology, Volumes I-IV (D. M. Weir and C. C. Blackwell eds 1986). (Sambrook et al. (1989) Molecular Cloning: A Laboratory Manual 2nd ed., Cold Spring Harbor Laboratory Press and Ausubel et al. Eds. (1997) Current Protocols in Molecular Biology, John Wiley & Sons, Inc.).

### Abbreviations

Standard abbreviations for nucleotides and amino acids are used in this specification.

| | |
|---|---|
| BCA | bicinchonic acid |
| BME | beta-mercapto-ethanol |
| BSA | Bovine serum albumin |
| EB | elementary body |
| EDTA | ethylenediaminetetraacetic acid |
| HtrA | high temperature requirement serine protease |
| IFU | Inclusion forming units |
| IPTG | Isopropyl-beta-D-thiogalactoside |
| LB | Luria Bertani (medium) |
| MES | 2(N-morpholino) ethane sulfonic acid |
| MHC | major histocompatibility complex |
| mLT | mutated E. coli heat-labile toxin (LT) |
| MOMP | Chlamydiaceae major outer membrane protein |
| PBS | phosphate buffered saline |
| PVDF | polyvinylidene difluroide |
| RB | Reticulate body |
| SSC | standard saline citrate |
| TBS | tris buffered saline |

### DEFINITIONS

It is to be noted that the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

The terms “nucleic acid” or “nucleic acid fragment” refers to any one or more nucleic acid segments, e.g., DNA or RNA fragments, present in a polynucleotide or construct. Two or more nucleic acids of the present invention can be present in a single polynucleotide construct, e.g., on a single plasmid, or in separate (non-identical) polynucleotide constructs, e.g., on separate plasmids. Furthermore, any nucleic acid or nucleic acid fragment may encode a single polypeptide, e.g., a single antigen, cytokine, or regulatory polypeptide, or may encode more than one polypeptide, e.g., a nucleic acid may encode two or more polypeptides. In addition, a nucleic acid may encode a regulatory element such as a promoter or a transcription terminator, or may encode a specialized element or motif of a polypeptide or protein, such as a secretory signal peptide or a functional domain.

The term “polynucleotide” is intended to encompass a single nucleic acid or nucleic acid fragment as well as plural nucleic acids or nucleic acid fragments, and refers to an isolated molecule or construct, e.g., a virus genome (e.g., a non-infectious viral genome), messenger RNA (mRNA), plasmid DNA (pDNA), or derivatives of pDNA (e.g., minicircles as described in (Darquet, A-M et al., Gene Therapy 4:1341-1349 (1997)) comprising a polynucleotide. A polynucleotide may be provided in linear (e.g., mRNA), circular (e.g., plasmid), or branched form as well as double-stranded or single-stranded forms. A polynucleotide may comprise a conventional phosphodiester bond or a non-conventional bond (e.g., an amide bond, such as found in peptide nucleic acids (PNA)).

As used herein, the term “polypeptide” is intended to encompass a singular “polypeptide” as well as plural “polypeptides,” and comprises any chain or chains of two or more amino acids. Thus, as used herein, terms including, but not limited to “peptide,” “dipeptide,” “tripeptide,” “protein,” “amino acid chain,” or any other term used to refer to a chain or chains of two or more amino acids, are included in the definition of a “polypeptide,” and the term “polypeptide” may be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

The term “C. trachomatis HtrA polypeptide,” as used herein, encompasses full length HtrA, serotypic, allelic, and other variants of full length HtrA, fragments of full length HtrA, serotypic, allelic, and other variants of fragments of full length HtrA, derivatives of full-length HtrA, derivatives of fragments of full-length HtrA, analogues of full-length HtrA, analogues of fragments of full-length HtrA, and chimeric and fusion polypeptides comprising full length HtrA or one or more fragments of full length HtrA.

The terms “fragment,” “analog,” “derivative,” or “variant” when referring to Chlamydia polypeptides of the present invention include any polypeptides which retain at least some of the immunogenicity or antigenicity of the naturally-occurring proteins. Fragments of Chlamydia polypeptides of the present invention include proteolytic fragments, deletion fragments and in particular, fragments of Chlamydia polypeptides which exhibit increased solubility during expression, purification, and or administration to an animal. Fragments of Chlamydia polypeptides further include proteolytic fragments or deletion fragments which exhibit reduced pathogenicity when delivered to a subject. Polypeptide fragments further include any portion of the polypeptide which comprises an antigenic or immunogenic epitope of the native polypeptide, including linear as well as three-dimensional epitopes.

An “epitopic fragment” of a polypeptide antigen is a portion of the antigen that contains an epitope. An “epitopic fragment” may, but need not, contain amino acid sequence in addition to one or more epitopes.

The term “variant,” as used herein, refers to a polypeptide that differs from the recited polypeptide due to amino acid substitutions, deletions, insertions, and/or modifications. Variants may occur naturally, such as a serotypic variant. The term “serotypic variant” is intended polypeptides or polynucleotides that are present in a different serotype of Chlamydia sp. of the present invention. The serotypic variants are naturally occurring variants, but it can also be produced using art-known mutagenesis techniques. In the case of C. trachomatis, at least 18 different strains, or “serotypic variants” (serovars) have been identified. There exists a high degree of variation between strains in some genes, for example, the genes encoding C. trachomatis HtrA polypeptide.

Non-naturally occurring variants may be produced using art-known mutagenesis techniques. In a preferred embodiment, variant polypeptides differ from an identified sequence by substitution, deletion or addition of five amino acids or fewer. Such variants may generally be identified by modifying a polypeptide sequence, and evaluating the antigenic properties of the modified polypeptide using, for example, the representative procedures described herein.

Polypeptide variants preferably exhibit at least about 60-70%, for example 75%, 80%, 85%, 90%, 94%, 95%, 96%, 97%, 98%, 99% or 99.9% sequence identity with identified polypeptides. Variant polypeptides may comprise conservative or non-conservative amino acid substitutions, deletions or additions. Derivatives of Chlamydia polypeptides of the present invention, are polypeptides which have been altered so as to exhibit additional features not found on the native polypeptide. Examples include fusion proteins. An analog is another form of a Chlamydia polypeptide of the present invention. An example is a proprotein which can be activated by cleavage of the proprotein to produce an active mature polypeptide.

Variants may also, or alternatively, contain other modifications, whereby, for example, a polypeptide may be conjugated or coupled, e.g., fused to a heterologous polypeptide, e.g., a signal (or leader) sequence at the N-terminal end of the protein which co-translationally or post-translationally directs transfer of the protein. The polypeptide may also be conjugated or produced coupled to a linker or other sequence for ease of synthesis, purification or identification of the polypeptide (e.g., 6-His), or to enhance binding of the polypeptide to a solid support. For example, a polypeptide may be conjugated or coupled to an immunoglobulin Fc region. The polypeptide may also be conjugated or coupled to a sequence that imparts or modulates the immune response to the polypeptide (e.g. a T-cell epitope, B-cell epitope, cytokine, chemokine, etc.) and/or enhances uptake and/or processing of the polypeptide by antigen presenting cells or other immune system cells. The polypeptide may also be conjugated or coupled to other polypeptides/epitopes from Chlamydia sp. and/or from other bacteria and/or other viruses to generate a hybrid immunogenic protein that alone or in combination with various adjuvants can elicit protective immunity to other pathogenic organisms.

The term “integer” as used herein refers to a number or numbers of either nucleic acids or amino acids. The term “integer” can be used to specify a range of nucleotide or peptide sequences. For example, amino acid sequences of amino acids a-483 of SEQ ID NO: 2, wherein a is any integer between 17 and 22, can be any one of the following: amino acids 17-483 of SEQ ID NO: 2; amino acids 18-483 of SEQ ID NO: 2; amino acids 19-483 of SEQ ID NO: 2; amino acids 20-483 of SEQ ID NO: 2; amino acids 21-483 of SEQ ID NO: 2; or amino acids 22-483 of SEQ ID NO: 2.

The term “sequence identity” as used herein refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window (e.g., SEQ ID NO: 2 and a homologous polypeptide from another C. trachomatis isolate). In order to optimally align sequences for comparison, the portion of a polynucleotide or polypeptide sequence in the comparison window may comprise additions or deletions termed gaps while the reference sequence (e.g. SEQ ID NO: 2) is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap dropoff (50), expect value (10) and any other required parameter including but not limited to matrix option.

The term “epitopes,” as used herein, refers to portions of a polypeptide having antigenic or immunogenic activity in an animal, for example a mammal, for example, a human. An “immunogenic epitope,” as used herein, is defined as a portion of a protein that elicits an immune response in an animal, as determined by any method known in the art. The term “antigenic epitope,” as used herein, is defined as a portion of a protein to which an antibody or T-cell receptor can immunospecifically bind its antigen as determined by any method well known in the art. Immunospecific binding excludes non-specific binding but does not necessarily exclude cross-reactivity with other antigens. Whereas all immunogenic epitopes are antigenic, antigenic epitopes need not be immunogenic.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, and the like, are outside the coding region.

The term “codon optimization” is defined herein as modifying a nucleic acid sequence for enhanced expression in the cells of the host of interest by replacing at least one, more than one, or a significant number, of codons of the native sequence with codons that are more frequently or most frequently used in the genes of that host. Various species exhibit particular bias for certain codons of a particular amino acid.

The term “pharmaceutical compositions” comprise compositions containing immunogenic peptides of the invention which are administered to an individual already suffering from a Chlamydia infection or an individual in need of immunization against Chlamydia infection.

The term “pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the polypeptide, polynucleotides, compositions, and vaccines of the present invention are pharmaceutically acceptable.

An “effective amount” is that amount the administration of which to an individual, either in a single dose or as part of a series, is effective for treatment or prevention. An amount is effective, for example, when its administration results in a reduced incidence of C. trachomatis in the lower genital tract relative to an untreated individual, as determined two weeks after challenge with infectious C. trachomatis. This amount varies depending upon the health and physical condition of the individual to be treated, the taxonomic group of individual to be treated (e.g. human, nonhuman primate, primate, etc.), the responsive capacity of the individual's immune system, the degree of protection desired, the formulation of the vaccine, a professional assessment of the medical situation, and other relevant factors. It is expected that the effective amount will fall in a relatively broad range that can be determined through routine trials. Typically a single dose is from about 10 μg to 10 mg/kg body weight of purified polypeptide or an amount of a modified carrier organism or virus, or a fragment or remnant thereof, sufficient to provide a comparable quantity of recombinantly expressed HtrA polypeptide. The term “peptide vaccine” or “subunit vaccine” refers to a composition comprising one or more polypeptides of the present invention, which when administered to an animal are useful in stimulating an immune response against Chlamydia infection.

The term “subject” is meant any subject, particularly a mammalian subject, for whom diagnosis, prognosis, immunization, or therapy is desired. Mammalian subjects include, but are not limited to, humans, domestic animals, farm animals, zoo animals such as bears, sport animals, pet animals such as dogs, cats, guinea pigs, rabbits, rats, mice, horses, cattle, bears, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the animal is a human subject.

The term “animal” is intended to encompass a singular “animal” as well as plural “animals” and comprises mammals and birds, as well as fish, reptiles, and amphibians. The term animal also encompasses model animals, e.g., disease model animals. In some embodiments, the term animal includes valuable animals, either economically or otherwise, e.g., economically important breeding stock, racing animals, show animals, heirloom animals, rare or endangered animals, or companion animals. In particular, the mammal can be a human subject, a food animal or a companion animal.

As used herein, an “subject in need thereof” refers to an individual for whom it is desirable to treat, i.e., to prevent, cure, retard, or reduce the severity of Chlamydia disease symptoms, and/or result in no worsening of Chlamydia disease over a specified period of time.

The terms “priming” or “primary” and “boost” or “boosting” as used herein to refer to the initial and subsequent immunizations, respectively, i.e., in accordance with the definitions these terms normally have in immunology. However, in certain embodiments, e.g., where the priming component and boosting component are in a single formulation, initial and subsequent immunizations may not be necessary as both the “prime” and the “boost” compositions are administered simultaneously.

The term “passive immunity” refers to the immunity to an antigen developed by a host animal, the host animal being given antibodies produced by another animal, rather than producing its own antibodies to the antigen. The term “active immunity” refers to the production of an antibody by a host animal as a result of the presence of the target antigen.

### Polynucleotides

The present invention includes a polynucleotide comprising a nucleic acid encoding a full-length or mature Chlamydia HtrA polypeptide or fragment, analog, derivative, or variant thereof. In one embodiment of the invention, the Chlamydia HtrA polypeptide or fragment, analog, derivative or variant thereof is a C. trachomatis HtrA polypeptide or fragment, analog, derivative or variant thereof. In another embodiment, the Chlamydia HtrA polypeptide or fragment, analog, derivative or variant thereof is a C. pneumoniae HtrA polypeptide or fragment, analog, derivative or variant thereof.

Polynucleotide sequences encoding Chlamydia HtrA polypeptides may be cloned from genomic DNA from any suitable Chlamydia strain (e.g., C. trachomatis or C. pneumoniae strain) by a variety of techniques known in the art. For instance, C. trachomatis can be cultured as an infected-cell culture as described in McClenaghan, et al., Infect. Immun. 45(2):384-389, 1984. Chlamydia genomic DNA can be isolated, for example, as described in Peeling, et al., Infect. Immun., 46(2):484-488, 1984. Full length HtrA genes may be cloned, for example, by PCR amplification. Suitable primers are described herein, or may be designed using techniques well known in the art. An oligonucleotide forward primer, encoding a known N-terminal sequence of Chlamydia HtrA, may be used together with an oligonucleotide reverse primer, which comprises the reverse complement of a known C-terminal sequence. Suitable primers may include a synthetic restriction enzyme cleavage site which is not found within the Chlamydia HtrA coding sequence, to facilitate manipulation of the PCR amplification clone. Suitable C. trachomatis forward primers include SEQ ID NO: 9, which includes a synthetic NcoI restriction site. Suitable reverse primers include SEQ ID NO: 10, which includes a synthetic SalI restriction site. Other suitable primers can be readily identified by one skilled in the art based on SEQ ID NO: 1 (C. trachomatis HtrA gene from serovar L₂), SEQ ID NO: 5 (a corresponding serotypic variant from serovar D) and SEQ ID NO: 7 (a corresponding serotypic variant from serovar MoPn). Alternatively, degenerate primers may be designed which permit amplification of corresponding genes despite a high degree of sequence variability. PCR conditions can also be modified to allow for a greater degree of sequence similarity, as is well known to those skilled in the art.

Alternatively, polynucleotide sequences encoding Chlamydia HtrA polypeptides, for instance, C. trachomatis HtrA polypeptides, may be obtained by screening an appropriate Chlamydia genomic library, for instance, C. trachomatis genomic library, using oligonucleotide or other polynucleotide probe sequences that encode portions of the HtrA amino acid sequence, or that represent complements of such coding sequences. Techniques to prepare genomic libraries are well known to a person of ordinary skill in the art. In the preparation of genomic libraries, DNA fragments are generated, some of which encode all or part of an HtrA polypeptide. To generate these fragments, genomic DNA can be cleaved, or partially cleaved, using specific restriction enzymes. Alternatively, DNAse in the presence of manganese may be used to fragment genomic DNA, or the DNA can be physically sheared, for example, by sonification. The genomic DNA fragments can then be separated according to size by standard techniques, including but not limited to, agarose or polyacrylamide gel electrophoresis, column chromatography, or sucrose gradient centrifugation. The genomic DNA fragments can then be inserted into suitable vectors, including but not limited to, plasmids, cosmids, bacteriophages lambda or T4, or yeast artificial chromosomes (YAC). The genomic library can then be screened by hybridization.

Alternatively, polynucleotide sequences encoding HtrA polypeptides may be obtained by screening an appropriate Chlamydia expression library. Techniques for preparation of expression libraries are well known to a person of ordinary skill in the art. For instance, DNA encoding polypeptides that are actually expressed is reverse transcribed from isolated C. trachomatis mRNA. These cDNAs are then isolated and ligated into an expression vector so that the inserted coding sequence will be expressed by a host cell into which the vector is introduced. Various screening assays can then be used to select for host cells expressing HtrA polypeptide. For example, antibodies raised against C. trachomatis HtrA can be used to identify desired clones using methods well known in the art. A cDNA sequence encoding HtrA can then be isolated from an identified expression clone.

Clones encoding C. trachomatis or C. pneumoniae HtrA polypeptides can be checked for completeness of coding sequence by a variety of techniques known in the art. For example, the cloned insert can be sequenced and its deduced, translated amino acid sequence compared with known sequences of full-length HtrAs is shown in SEQ ID NOs:2, 4, 6 or 8. Where the isolated clone appears to be only partial, complete clones may be isolated using the insert of the partial clone as a hybridization probe for further screening. Alternatively, a complete coding sequence can be reconstructed using contig assembly of overlapping partial clones, splicing overlapping partial clones with appropriate restriction enzymes at suitable shared restriction sites, and ligating the spliced inserts together.

Included within the scope of the invention are polynucleotides at least 60%, 70%, 75%, 80%, 85%, 90%, 95%, 96%, 97%, 98%, 99% or 100% identical to polynucleotides encoding C. trachomatis or C. pneumoniae HtrA polypeptides, fragments, derivatives, or variants thereof. Variants commonly occur with many genes of simple, unicellular organisms. Examples of variants are serotypic variants. Another example of variants can be found within strains. For example, even within strains, different isolates obtained from different patients and/or geographical locations can exhibit some variation. Coding nucleotide sequences and deduced amino acid sequences of outermembrane proteins can have as little as 60% sequence identity, between strains of C. trachomatis. (Yuan, et al., Infect. Immun., 57(4):1040-1049, 1989; Moroni, et al., Arch Microbiol, 165:164-168, 1996; Gomes, et al., J. Bacteriol., 186(13):4295-4306, 2004; Millman, et al., J. Bacteriol., 186(8):2457-2465, 2004).

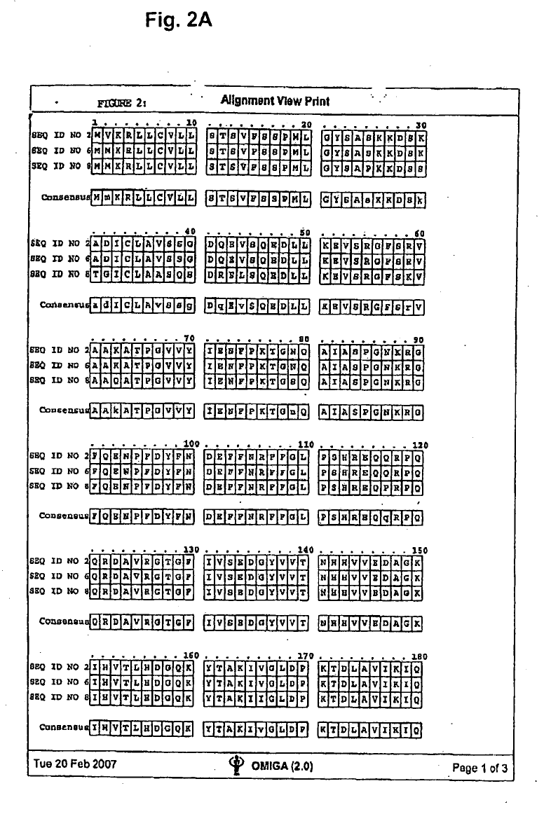

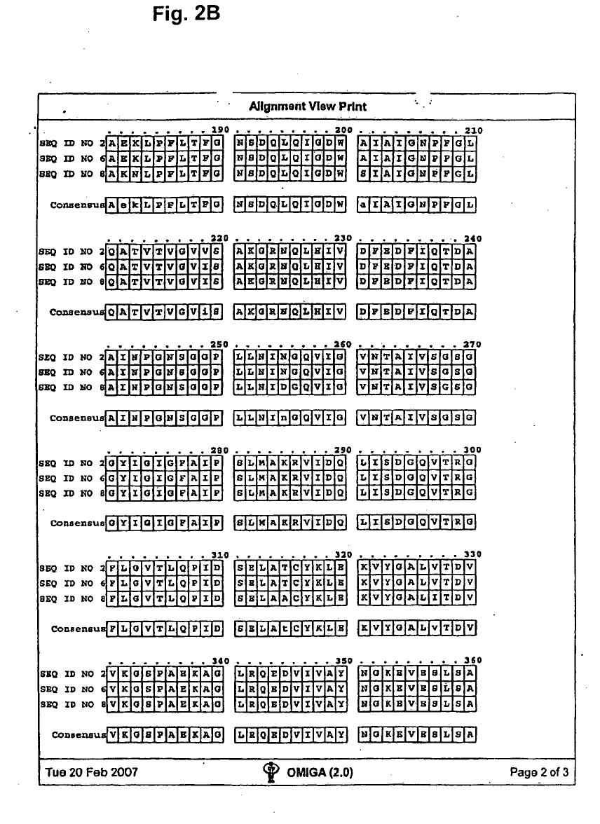

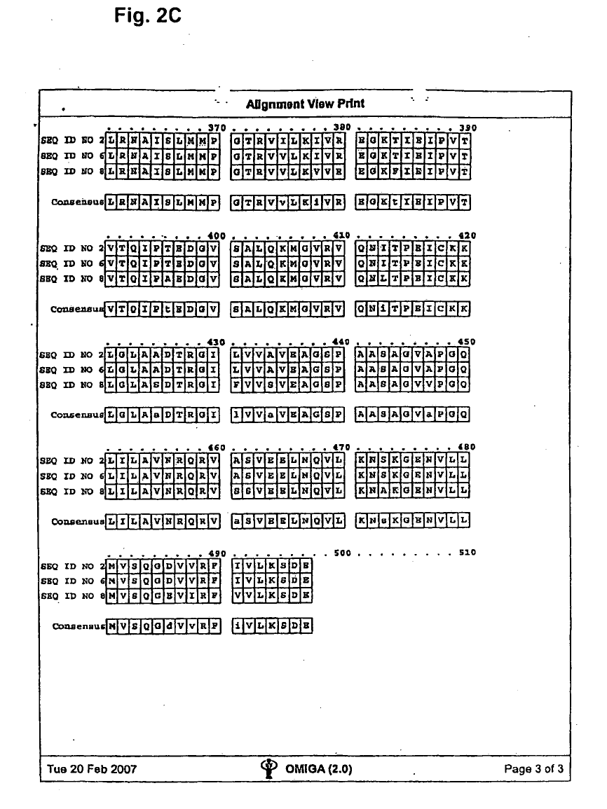

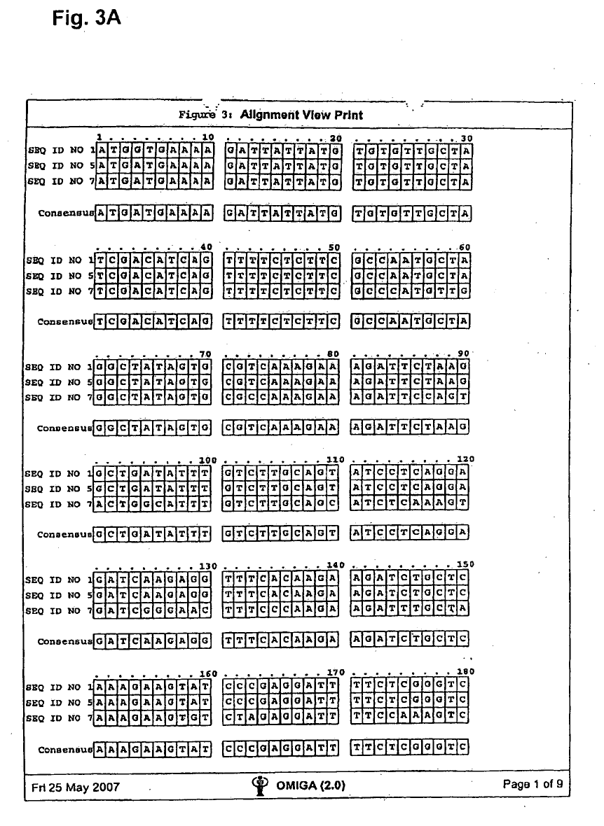

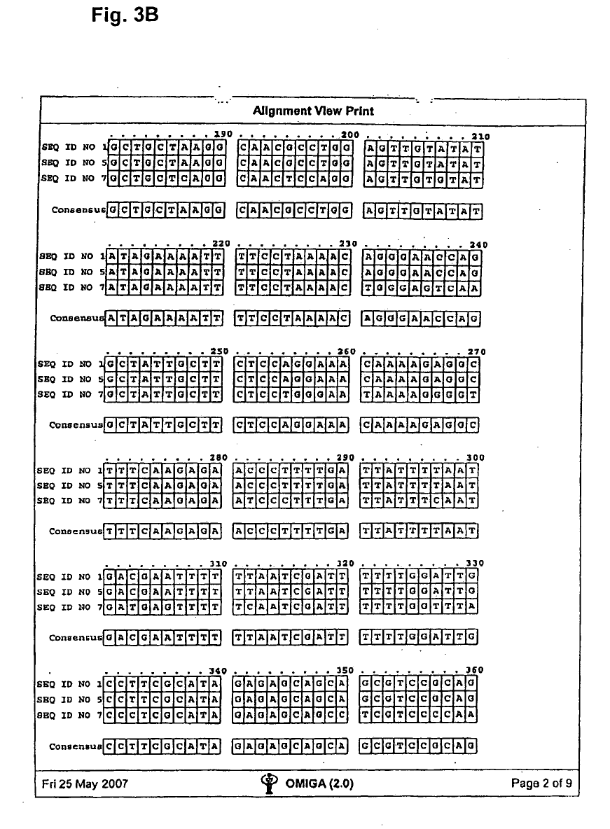

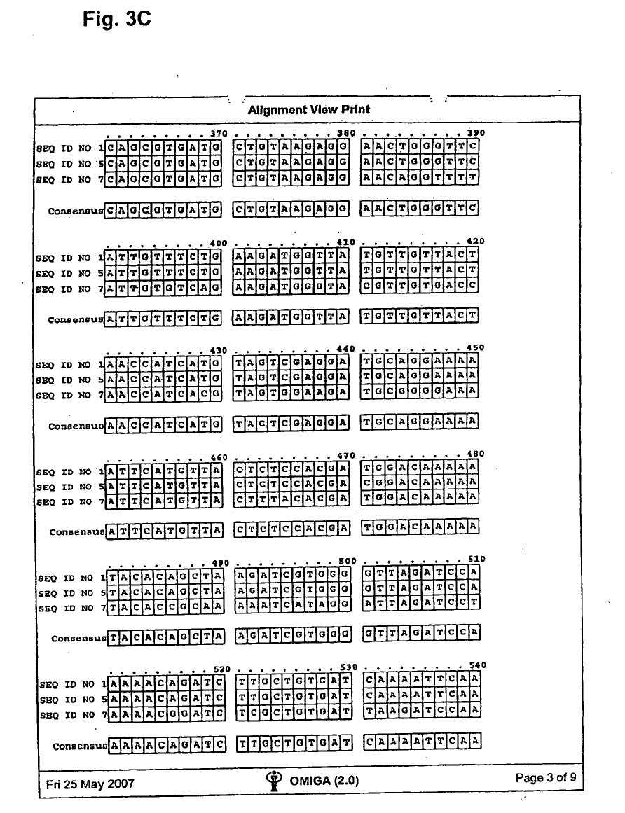

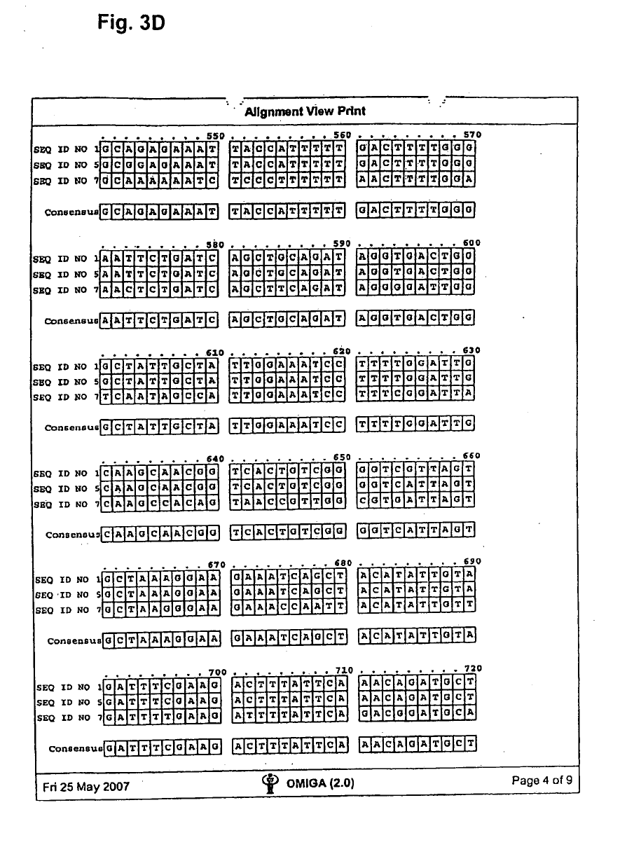

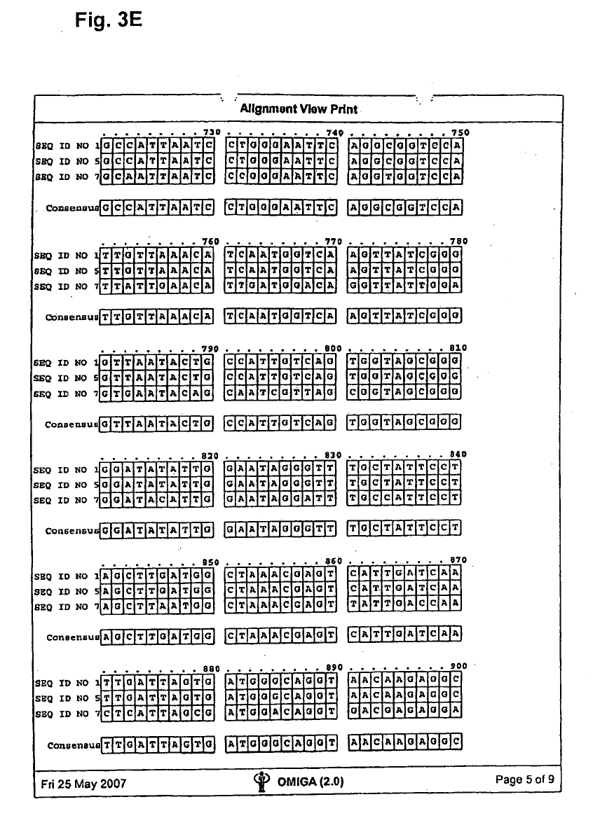

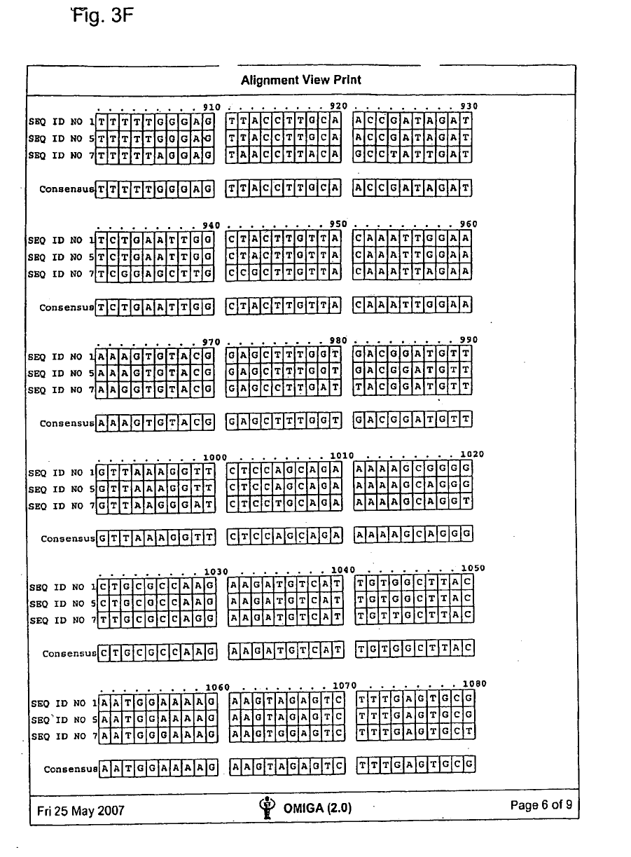

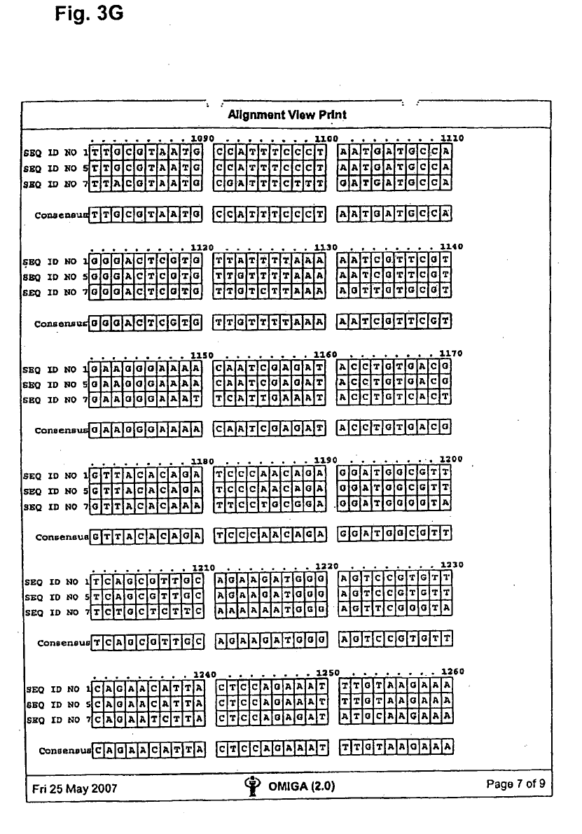

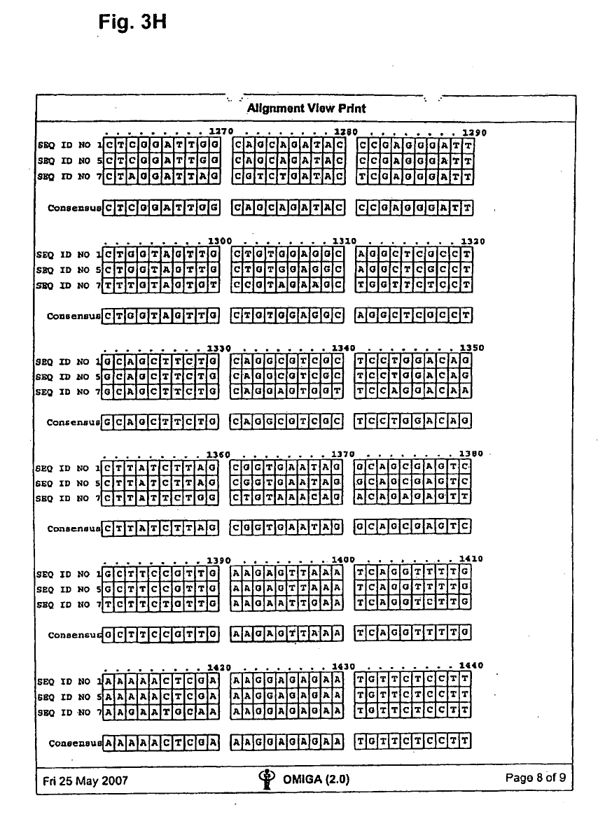

The sequences of genes encoding C. trachomatis HtrA are known from three different serovars. The nucleotide sequence from the L₂ serovar is disclosed here as SEQ ID NO: 1. SEQ ID NO: 2 is the amino acid sequence of C. trachomatis HtrA polypeptide L₂ serovar. Also known are serotypic variants from the D (Stephens, et al., Science, 282(5389):754-9, 1998) and MoPn (Read, et al., Nucleic Acids Res., 28(6):1397-406, 2000) serovars, SEQ ID NO: 5 and SEQ ID NO: 7, respectively. The corresponding translated polypeptide sequences from the L₂, D and MoPn serovars are given respectively by SEQ ID NO: 2, SEQ ID NO: 6 and SEQ ID NO: 8. As shown in Table 1, polynucleotide sequence identities of C. trachomatis HtrA genes vary by more than 17% between serovars while amino acid sequence identities vary by more than 7%. See also FIGS. 2 and 3.

TABLE 1
Serotypic variation of C. trachomatis HtrA genes

| | Serotype L₂ | Serotype D | Serotype MoPn |
|---|---|---|---|
| DNA | 100% | 99.6% | 82.1% |
| Protein | 100% | 99.4% | 92.8% |

Shown are percent sequence identity values between HtrA genes from three C. trachomatis serovars. All comparisons are vs HtrA from the L₂ serovar. Both coding nucleotide and translated amino acid sequence comparisons are shown in FIGS. 2 and 3, respectively.

Serotypic variation of HtrA is much lower than that observed with MOMP. Serotypic variants are therefore expected to be immunologically cross-reactive. The present invention thus includes HtrA polypeptides found in other Chlamydia sp, e.g., C. trachomatis serotypes such as, but not limited to, A, B, Ba, C, D, E, F, G, H, I, J, K, L₁, L₂, L₃, MoPn, or Chlamydia pneumoniae or fragments thereof. These proteins can be obtained using the methods described above or available to a person of ordinary skill in the art.

For instance, the polypeptide sequences of C. pneumoniae are publically available. Exemplary C. pneumoniae HtrA polypeptide sequences are provided in the Table 2. C. pneumoniae HtrA polypeptides can be modified by the methods disclosed herein, for instance, to reduce protease activity.

TABLE 2
C. pneumoniae HtrA polypeptide sequences

| C. pneumoniae HtrA | Amino Acid Sequence |
|---|---|
| C. pneumoniae TW- | MRSWLAVLVGSGLLALPLSGQAVGKKES |
| 183 HtrA | RVSELPQDVLLKEISGGFSKVATKATPA |
| (SEQ ID NO.: 31) | VVYIESFPKSQAVTHPSPGRRGPYENPF |
| | DYFNDEFFNRFFGLPSQREKPQSKEAVR |
| | GTGFLVSPDGYIVTNNHVVEDTGKIHVT |
| | LHDGQKYPATVIGLDPKTDLAVIKIKSQ |
| | NLPYLSFGNSDHLKVGDWAIAIGNPFGL |
| | QATVTVGVISAKGRNQLHIADFEDFIQT |
| | DAAINPGNSGGPLLNIDGQVIGVNTAIV |
| | SGSGGYIGIGFAIPSLMANRIIDQLIRD |
| | GQVTRGFLGVTLQPIDAELAACYKLEKV |
| | YGALVTDVVKGSPADKAGLKQEDVIIAY |
| | NGKEVDSLSMFRNAVSLMNPDTRIVLKV |
| | VREGKVIEIPVTVSQAPKEDGMSALQRV |
| | GIRVQNLTPETAKKLGIAPETKGILIIS |
| | VEPGSVAASSGIAPGQLILAVNRQKVSS |
| | IEDLNRTLKDSNNENILLMVSQGDVIRF |
| | IALKPEE |
| C. pneumoniae TW- | TTGCGTTCGTGGCTAGCTGTACTTGTTG |
| 183 HtrA nucleotide | GTTCAGGTCTGCTAGCTCTTCCTTTATC |
| sequence | AGGGCAAGCTGTCGGGAAAAAAGAATCC |
| (SEQ ID NO.: 32) | TGAGTTTCCGAGCTGCCTCAAGACGTTC |
| | TTCTTAAAGAGATCTCGGGAGGGTTTTC |
| | TAAGGTCGCTACCAAGGCGACTCCCGCT |
| | GTTGTGTACATAGAAAGTTTCCCAAAGA |
| | GCCAGGCTGTAACACATCCTTCTCCTGG |
| | ACGCCGTGGGCCTTATGAAAATCCTTTT |
| | GATTATTTTAATGATGAGTTTTTCAATC |
| | GTTTTTTTGGTCTACCTTCACAGAGGGA |
| | AAAACCTCAAAGTAAAGAGGCGGTTCGA |
| | GGAACAGGTTTCCTAGTATCTCCAGATG |
| | GCTATATTGTGACTAATAACCATGTTGT |
| | CGAAGATACAGGTAAGATTCACGTAACT |
| | CTTCATGATGGGCAAAAGTACCCAGCAA |
| | CTGTAATCGGACTCGATCCTAAAACAGA |
| | CCTTGCAGTCATTAAAATTAAATCCCAA |
| | AACCTCCCGTATCTTTCTTTTGGAAACT |
| | CCGACCACTTAAAAGTCGGAGATTGGGC |
| | AATTGCAATTGGAAATCCCTTCGGTCTT |
| | CAAGCTACGGTCACCGTAGGTGTCATCA |
| | GTGCTAAAGGAAGAAATCAACTCCACAT |
| | TGCAGATTTTGAAGATTTTATTCAGACA |
| | GATGCTGCGATTAATCCAGGCAACTCTG |
| | GAGGCCCTCTTCTAAATATTGATGGACA |
| | GGTCATCGGTGTTAATACTGCCATTGTC |
| | AGTGGTAGTGGTGGCTATATTGGAATCG |
| | GGTTTGCGATTCCTAGCCTTATGGCAAA |
| | TAGAATCATAGATCAGCTGATTCGTGAT |
| | GGTCAAGTTACCCGAGGATTCTTAGGAG |
| | TGACTTTACAACCTATAGATGCGGAACT |
| | CGCTGCTTGCTACAAACTCGAAAAGGTT |
| | TATGGCGCTTTAGTCACAGATGTTGTTA |
| | AAGGATCTCCAGCAGATAAAGCAGGGCT |
| | AAAACAAGAAGATGTGATCATTGCTTAT |
| | AATGGGAAAGAAGTCGATTCACTGAGTA |
| | TGTTCCGTAATGCTGTTTCTTTAATGAA |
| | TCCAGATACACGTATTGTTCTAAAGGTA |
| | GTTCGTGAAGGAAAGGTTATCGAAATAC |
| | CCGTGACAGTTTCTCAAGCTCCAAAAGA |
| | AGATGGAATGTCGGCTTTACAGCGTGTG |
| | GGAATCCGTGTGCAAAACCTAACTCCTG |
| | AAACTGCTAAGAAGCTGGGAATTGCTCC |
| | AGAGACTAAAGGCATTTTGATTATAAGT |
| | GTTGAACCAGGGTCTGTAGCAGCTTCTT |
| | CAGGAATTGCTCCTGGTCAGCTGATCCT |
| | TGCTGTGAATAGACAAAAAGTATCTTCG |
| | ATTGAAGATCTGAATAGAACGTTAAAAG |
| | ATTCTAACAATGAGAATATTCTTCTTAT |
| | GGTTTCTCAAGGAGATGTTATTCGCTTC |
| | ATTGCCCTGAAACCTGAAGAATAA |
| C. pneumoniae | MITKQLRSWLAVLVGSSLLALPLSGQAV |
| J138 and CWL029 | GKKESRVSELPQDVLLKEISGGFSKVAT |
| HtrA | KATPAVVYIESFPKSQAVTHPSPGRRGP |
| (SEQ ID NO.: 33) | YENPFDYFNDEFFNRFFGLPSQREKPQS |
| | KEAVRGTGFLVSPDGYIVTNNHVVEDTG |
| | KIHVTLHDGQKYPATVIGLDPKTDLAVI |
| | KIKSQNLPYLSFGNSDHLKVGDWAIAIG |
| | NPFGLQATVTVGVISAKGRNQLHIADFE |
| | DFIQTDAAINPGNSGGPLLNIDGQVIGV |
| | NTAIVSGSGGYIGIGFAIPSLMANRIID |
| | QLIRDGQVTRGFLGVTLQPIDAELAACY |
| | KLEKVYGALVTDVVKGSPADKAGLKQED |
| | VIIAYNGKEVDSLSMFRNAVSLMNPDTR |
| | IVLKVVREGKVIEIPVTVSQAPKEDGMS |
| | ALQRVGIRVQNLTPETAKKLGIAPETKG |
| | ILIISVEPGSVAASSGIAPGQLILAVNR |
| | QKVSSIEDLNRTLIMSNNENILLMVSQG |
| | DVIRFIALKPEE |
| C. pneumoniae | ATGATAACTAAGCAATTGCGTTCGTGGC |
| J138 and CWL029 | TAGCTGTACTTGTTGGTTCAAGTCTGCT |
| HtrA nucleotide | AGCTCTTCCTTTATCAGGGCAAGCTGTC |
| sequence | GGGAAAAAAGAATCTCGAGTTTCCGAGC |
| (SEQ ID NO.: 34) | TGCCTCAAGACGTTCTTCTTAAAGAGAT |
| | CTCGGGAGGGTTTTCTAAGGTCGCTACA |
| | ACGGCGACTCCCGCTGTTGTGTACATAG |
| | AAAGTTTCCCAAAGAGCCAGGCTGTAAC |
| | ACATCCTTCTCCTGGACGCCGTGGGCCT |
| | TATGAAAATCCTTTTGATTATTTTAATG |
| | ATGAGTTTTTCAATCGTTTTTTTGGTCT |
| | ACCTTCACAGAGGGAAAAACCTCAAAGT |
| | AAAGAGGCGGTTCGAGGAACAGGTTTCC |
| | TAGTATCTCCAGATGGCTATATTGTGAC |
| | TAATAACCATGTTGTCGAAGATACAGGT |
| | AAGATTCACGTAACTCTTCATGATGGGC |
| | AAAAGTACCCAGCAACTGTAATCGGACT |
| | CGATCCTAAAACAGACCTTGCAGTCATT |
| | AAAATTAAATCCCAAAACCTCCCGTATC |
| | TTTCTTTTGGAAACTCCGACCACTTAAA |
| | AGTCGGAGATTGGGCAATTGCAATTGGA |
| | AATCCCTTCGGTCTTCAAGCTACGGTCA |
| | CCGTAGGTGTCATCAGTGCTAAAGGAAG |
| | AAATCAACTCCACATTGCAGATTTTGAA |
| | GATTTTATTCAGACAGATGCTGCGATTA |
| | ATCCAGGCAACTCTGGAGGCCCTCTTCT |
| | AAATATTGATGGACAGGTCATCGGTGTT |
| | AATACTGCCATTGTCAGTGGTAGTGGTG |
| | GCTATATTGGAATCGGGTTTGCGATTCC |
| | TAGCCTTATGGCAAATAGAATCATAGAT |
| | CAGCTGATTCGTGATGGTCAAGTTACCC |
| | GAGGATTCTTAGGAGTGACTTTACAACC |
| | TATAGATGCGGAACTCGCTGCTTGCTAC |
| | AAACTCGAAAAGGTTTATGGCGCTTTAG |
| | TCACAGATGTTGTTAAAGGATCTCCAGC |
| | AGATAAAGCAGGGCTAAAACAAGAAGAT |
| | GTGATCATTGCTTATAATGGGAAAGAAG |
| | TCGATTCACTGAGTATGTTCCGTAATGC |
| | TGTTTCTTTAATGAATCCAGATACACGT |
| | ATTGTTCTAAAGGTAGTTCGTGAAGGAA |
| | AGGTTATCGAAATACCCGTGACAGTTTC |
| | TCAAGCTCCAAAAGAAGATGGAATGTCG |
| | GCTTTACAGCGTGTGGGAATCCGTGTGC |
| | AAAACCTAACTCCTGAAACTGCTAAGAA |
| | GCTGGGAATTGCTCCAGAGACTAAAGGC |
| | ATTTTGATTATAAGTGTTGAACCAGGGT |
| | CTGTAGCAGCTTCTTCAGGAATTGCTCC |
| | TGGTCAGCTGATCCTTGCTGTGAATAGA |
| | CAAAAAGTATCTTCGATTGAAGATCTGA |
| | ATAGAACGTTAAAAGATTCTAACAATGA |
| | GAATATTCTTCTTATGGTTTCTCAAGGA |
| | GATGTTATTCGCTTCATTGCCCTGAAAC |
| | CTGAAGAATAA |
| C. pneumoniae | MITKQLRSWLAVLVGSXLLALPLSGQAV |
| AR39 HtrA | GKKESRVSELPQDVLLKEISGGFSKVAT |
| (SEQ ID NO.: 35) | KATPAVVYIESFPKSQAVTHPSPGRRGP |
| | YENPFDYFNDEFFNRFFGLPSQREKPQS |
| | KEAVRGTGFLVSPDGYIVTNNHVVEDTG |
| | KIHVTLHDGQKYPATVIGLDPKTDLAVI |
| | KIKSQNLPYLSFGNSDHLKVGDWAIAIG |
| | NPFGLQATVTVGVISAKGRNQLHIADFE |
| | DFIQTDAAINPGNSGGPLLNIDGQVIGV |
| | NTAIVSGSGGYIGIGFAIPSLMANRIID |
| | QLIRDGQVTRGELGVTLQPIDAELAACY |
| | KLEKVYGALVTDVVKGSPADKAGLKQED |
| | VIIAYNGKEVDSLSMFRNAVSLMNPDTR |
| | IVLKVVREGKVIEIPVTVSQAPKEDGMS |
| | ALQRVGIRVQNLTPETAKKLGIAPETKG |
| | ILIISVEPGSVAASSGIAPGQLILAVNR |
| | QKVSSIEDLNRTLKDSNNENILLMVSQG |
| | DVIRFIALKPEE |
| C. pneumoniae | ATGATAACTAAGCAATTGCGTTCGTGGC |
| AR39 HtrA | TAGCTGTACTTGTTGGTTCARGTCTGCT |
| nucleotide | AGCTCTTCCTTTATCAGGGCAAGCTGTC |
| sequence | GGGAAAAAAGAATCTCGAGTTTCCGAGC |
| (SEQ ID NO.: 36) | TGCCTCAAGACGTTCTTCTTAAAGAGAT |
| | CTCGGGAGGGTTTTCTAAGGTCGCTACC |
| | AAGGCGACTCCCGCTGTTGTGTACATAG |
| | AAAGTTTCCCAAAGAGCCAGGCTGTAAC |
| | ACATCCTTCTCCTGGACGCCGTGGGCCT |
| | TATGAAAATCCTTTTGATTATTTTAATG |
| | ATGAGTTTTTCAATCGTTTTTTTGGTCT |
| | ACCTTCACAGAGGGAAAAACCTCAAAGT |
| | AAAGAGGCGGTTCGAGGAACAGGTTTCC |
| | TAGTATCTCCAGATGGCTATATTGTGAC |
| | TAATAACCATGTTGTCGAAGATACAGGT |
| | AAGATTCACGTAACTCTTCATGATGGGC |
| | AAAAGTACCCAGCAACTGTAATCGGACT |
| | CGATCCTAAAACAGACCTTGCAGTCATT |
| | AAAATTAAATCCCAAAACCTCCCGTATC |
| | TTTCTTTTGGAAACTCCGACCACTTAAA |
| | AGTCGGAGATTGGGCAATTGCAATTGGA |
| | AATCCCTTCGGTCTTCAAGCTACGGTCA |
| | CCGTAGGTGTCATCAGTGCTAAAGGAAG |
| | AAATCAACTCCACATTGCAGATTTTGAA |
| | GATTTTATTCAGACAGATGCTGCGATTA |
| | ATCCAGGCAACTCTGGAGGCCCTCTTCT |
| | AAATATTGATGGACAGGTCATCGGTGTT |
| | AATACTGCCATTGTCAGTGGTAGTGGTG |
| | GCTATATTGGAATCGGGTTTGCGATTCC |
| | TAGCCTTATGGCAAATAGAATCATAGAT |
| | CAGCTGATTCGTGATGGTCAAGTTACCC |
| | GAGGATTCTTAGGAGTGACTTTACAACC |
| | TATAGATGCGGAACTCGCTGCTTGCTAC |
| | AAACTCGAAAAGGTTTATGGCGCTTTAG |
| | TCACAGATGTTGTTAAAGGATCTCCAGC |
| | AGATAAAGCAGGGCTAAAACAAGAAGAT |
| | GTGATCATTGCTTATAATGGGAAAGAAG |
| | TCGATTCACTGAGTATGTTCCGTAATGC |
| | TGTTTCTTTAATGAATCCAGATACACGT |
| | ATTGTTCTAAAGGTAGTTCGTGAAGGAA |
| | AGGTTATCGAAATACCCGTGACAGTTTC |
| | TCAAGCTCCAAAAGAAGATGGAATGTCG |
| | GCTTTACAGCGTGTGGGAATCCGTGTGC |
| | AAAACCTAACTCCTGAAACTGCTAAGAA |
| | GCTGGGAATTGCTCCAGAGACTAAAGGC |
| | ATTTTGATTATAAGTGTTGAACCAGGGT |
| | CTGTAGCAGCTTCTTCAGGAATTGCTCC |
| | TGGTCAGCTGATCCTTGCTGTGAATAGA |
| | CAAAAAGTATCTTCGATTGAAGATCTGA |
| | ATAGAACGTTAAAAGATTCTAACAATGA |
| | GAATATTCTTCTTATGGTTTCTCAAGGA |
| | GATGTTATTCGCTTCATTGCCCTGAAAC |
| | CTGAAGAATAA |

The present invention is directed to the polynucleotide encoding full-length or mature HtrA polypeptides, analogs, variants, as well as fragments thereof described herein. Full-length HtrA consists of a signal sequence, a catalytic domain (serine protease domain), PDZ 1 domain, and PDZ 2 domain. Mature HtrA is the full-length HtrA without a signal sequence. As one of skill in the art will appreciate, the beginning and ending residues of the domains may vary depending upon the computer modeling program used or the method used for determining the domains.

Three structural domains of C. trachomatis HtrA comprising the conserved regions can thus be identified—a solvent-protected serine protease domain and two highly solvent accessible PDZ interaction domains. Examples of predicted domains of SEQ ID NO: 2 are shown in Table 3.

TABLE 3
C. trachomatis HtrA Domains (SEQ ID NOs: 2, 4, 6, and 8)

| | Signal | | | PDZ 2 |
|---|---|---|---|---|
| Programs | Seq. | Catalytic domain | PDZ 1 domain | domain |
| UniProtKB/ | 1-16aa | 128-289aa | 290-381aa | 394-485aa |
| SwissProt | | | | |
| Pfam | 1-21aa | 128-288aa | 290-379aa | 428-483aa |
| SMART | 1-21aa | 110-288aa | 300-382aa | 404-486aa |
| PROSITE | n/a | n/a | 291-381aa | 407-485aa |
| NCBI | 1-16aa | 95-297aa | 300-389aa | 404-493aa |

The signal sequence can be deduced to be, e.g., about either amino acids 1-21 or about 1-16 of SEQ ID NOs: 2, 4, 6, or 8 or intermediate fragments; the serine protease domain can be deduced to be, e.g., about amino acids 128-288 or about amino acids 95-297 of SEQ ID NOs: 2, 4, 6, or 8 or intermediate fragments; PDZ 1 domain can be either about amino acids 300-379 or about amino acids 290-389 of SEQ ID NOs: 2, 4, 6, or 8 or intermediate fragments; and PDZ 2 domain can be about either amino acids 428-483 or about amino acids 394-493 of SEQ ID NOs: 2, 4, 6, or 8 or intermediate fragments. Similar domains may be identified for the HtrA of C. pneumoniae (SEQ ID NOs: 31, 33, 35, and 37).

The present invention includes a polynucleotide comprising, consisting essentially of, or consisting of a nucleic acid encoding an amino acid sequence at least 70%, 75%, 80%, 85%, 90%, 95%, 96%, 97%, 98%, 99%, or 100% identical to a reference amino acid sequence selected from the group consisting of: amino acids a-483 of SEQ ID NO: 2; amino acids 22-b of SEQ ID NO: 2; amino acids a-b of SEQ ID NO: 2; amino acids c-379 of SEQ ID NO: 2; amino acids 300-d of SEQ ID NO: 2; amino acids c-d of SEQ ID NO: 2; amino acids e-483 of SEQ ID NO: 2; amino acids 428-b of SEQ ID NO: 2; amino acids e-b of SEQ ID NO: 2; amino acids c-483 of SEQ ID NO: 2; amino acids 300-b of SEQ ID NO: 2; amino acids c-b of SEQ ID NO: 2; amino acids f-288 of SEQ ID NO: 2; amino acids 128-g of SEQ ID NO: 2; amino acids f-g of SEQ ID NO: 2; amino acids f-379 of SEQ ID NO: 2; amino acids 128-d of SEQ ID NO: 2; amino acids f-d of SEQ ID NO: 2; amino acids f-483 of SEQ ID NO: 2; amino acids 128-b of SEQ ID NO: 2; amino acids f-b of SEQ ID NO: 2; amino acids a-379 of SEQ ID NO: 2; amino acids 22-d of SEQ ID NO: 2; amino acids a-d of SEQ ID NO: 2; amino acids a-288 of SEQ ID NO: 2; amino acids 22-g of SEQ ID NO: 2; and amino acids a-g of SEQ ID NO: 2; wherein a is any integer between 17 and 22, b is any integer between 483-497, c is any integer between 290-300, d is any integer between 379-389, e is any integer between 394-428, f is any integer between 95-128, and g is any integer between 288 and 297. In another embodiments, the present invention is directed to the polynucleotides encoding the HtrA polypeptides, fragments, derivatives, analogs, or variants thereof, which lack a nucleic acid sequence encoding an amino acid sequence selected from the group consisting of: amino acids f-288 of SEQ ID NO: 2; amino acids 128-g of SEQ ID NO: 2; amino acids f-g of SEQ ID NO: 2; amino acids c-379 of SEQ ID NO: 2; amino acids 300-d of SEQ ID NO: 2; and amino acids c-d of SEQ ID NO: 2, wherein c is any integer between 290-300, d is any integer between 379-389, f is any integer between 95-128, and g is any integer between 288-297.

In certain embodiments, the present invention includes a polynucleotide comprising, consisting essentially of, or consisting of a nucleic acid sequence at least 60%, 70%, 75%, 80%, 85%, 90%, 95%, 96%, 97%, 98%, 99%, or 100% identical to a reference nucleic acid sequence encoding an amino acid sequence selected from the group consisting of: amino acids 22-483 of SEQ ID NO: 2; amino acids 17-483 of SEQ ID NO: 2; amino acids 22-497 of SEQ ID NO: 2; amino acids 17-497 of SEQ ID NO: 2; amino acids 290-381 of SEQ ID NO: 2; amino acids 290-379 of SEQ ID NO: 2; amino acids 300-382 of SEQ ID NO: 2; amino acids 291-381 of SEQ ID NO: 2; amino acids 300-389 of SEQ ID NO: 2; amino acids 394-485 of SEQ ID NO: 2; amino acids 428-483 of SEQ ID NO: 2; amino acids 404-486 of SEQ ID NO: 2; amino acids 407-485 of SEQ ID NO: 2; amino acids 404-493 of SEQ ID NO: 2; amino acids 290-485 of SEQ ID NO: 2; amino acids 290-483 of SEQ ID NO: 2; amino acids 300-486 of SEQ ID NO: 2; amino acids 291-485 of SEQ ID NO: 2; amino acids 300-493 of SEQ ID NO: 2; amino acids 128-381 of SEQ ID NO: 2; amino acids 128-379 of SEQ ID NO: 2; amino acids 110-382 of SEQ ID NO: 2; amino acids 95-389 of SEQ ID NO: 2; amino acids 128-485 of SEQ ID NO: 2; amino acids 128-483 of SEQ ID NO: 2; amino acids 110-486 of SEQ ID NO: 2; and amino acids 95-493 of SEQ ID NO: 2.

In certain embodiments, a polynucleotide comprises, consists essentially of, or consists of a nucleic acid sequence of SEQ ID NO: 1 or fragment thereof, which encodes the polypeptide sequences of the present invention. For example, the nucleotide sequence can be residues Y₁-1491 of SEQ ID NO: 1, wherein Y₁ is an integer between 49 and 64.

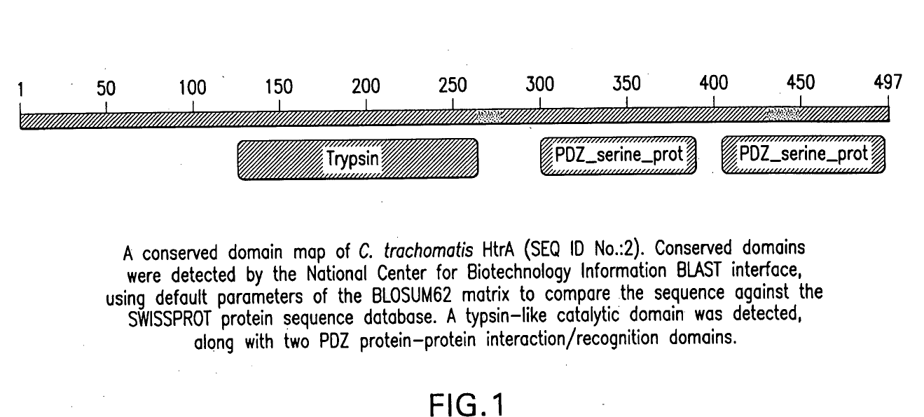

Alternatively, the domains of HtrA can be determined by comparing the C. trachomatis HtrA polypeptide sequence or C. pneumoniae HtrA polypeptide sequence to E. coli HtrA polypeptide sequence. In crystal structures, E. coli HtrA monomers have three distinct structural domains—a serine protease domain (about residues 17-259), and two PDZ protein-protein interaction domains (about residues 260-358 and 359-448). See FIG. 1. In E. coli, mouse and human HtrAs, these PDZ interaction domains provide broad substrate specificity, which is required for recognition of misfolded proteins. (Murwantoko, M., et al., Biochem. J., 381:895-904, 2004; Spiers, A., et al., J. Biological Chem., 277(42):39443-39449, 2002; Martins, L., et al., J. Biological Chem., 278(49):49417-49427, 2003). In crystal structures, E. coli HtrA forms hexamers. (Krojer, T., et al., Nature, 416:455-459, 2002). In these hexameric oligomers, HtrA serine protease domains are entirely shielded from solvent. In contrast, the PDZ domains are entirely solvent-exposed in monomers as well as hexamers. (Krojer, T., et al., Nature, 416:455-459, 2002).

The portion of C. trachomatis HtrA comprising about residues 127-497 has about 63% sequence identity with E. coli HtrA residues 114-474, as revealed by BLAST 2 SEQUENCES comparison using default parameters. A BLASTP comparison of C. trachomatis HtrA (SEQ ID NO: 2) against the SWISSPROT protein sequence database identifies a conserved trypsin-like catalytic domain (residues 135-273) along with two PDZ protein-protein interaction/recognition domains (residues 300-392 and 406-492). A conserved domain map of C. trachomatis HtrA is shown in FIG. 1.

Additional HtrA fragments of the present invention can be identified using a protease, e.g. cathepsin S. As shown in Table 4, Cathepsin S cleavage sites (Carmicle, S., et al., J. Biol. Chem., 227(1):155-160, 2002; Bromme, D., et al, Biochem J., 264(2):475-81, 1989; Plüger, E., et al., Eur. J. Immunol., 32:467-476, 2002) were identified in each of the three domains of C. trachomatis HtrA. Cathepsin S, a cysteine endoprotease, is expressed uniquely in antigen presenting cells and plays an important, and often critical, role in MHC Class II antigen processing. (Watts, C., Nature Immun., 5(7):685-692, 2004; Bromme, D., et al., Biochem J., 264(2):475-81, 1989; Plüger, E., et al., Eur. J. Immunol., 32:467-476, 2002). Initial proteolytic degradation of MHC Class II antigens is directed at solvent-exposed sites in the substrate protein, at linker regions between domains and at regions of structural disorder. (Carmicle, S., et al., J. Biol. Chem., 227(1):155-160, 2002; Novotny, J., et al., FEBS Lett., 211(2):185-9, 1987). Six Cathepsin S sites were identified in all. Sites 1, 2, 3 and 4 are within the solvent protected serine protease domain, i.e., about amino acids 17-299 of SEQ ID NO: 2. Site 5 is within the first solvent-exposed PDZ domain, i.e., about amino acids 300-392 of SEQ ID NO: 2. Site 6 is within the second solvent-exposed PDZ domain, i.e., about amino acids 393-497 of SEQ ID NO: 2. Table 4 shows Cathepsin S cleavage sites in C. trachomatis HtrA. Each cleavage site is identified by number, underlined, and expressed in bold face. Domain boundaries are also identified. The fragments that are produced by Cathepsin S include: amino acids 1-19; amino acids 20-497; amino acids 1-79; amino acids 80-497; amino acids 1-112; amino acids 113-497; amino acids 1-187; amino acids 188-497; amino acids 1-326; amino acids 327-497; amino acids 1-436; or amino acids 437-497 of SEQ ID NOs: 2, 6, or 8.

TABLE 4
Cathepsin S cleavage sites in C. trachomatis HtrA
[Serine protease domain (residues 1-299)]:
MVKRLLCVLLSTSVFSSPML¹GYSASKKDSKADICLAVSSGDQEVSQ
EDLLKEVSRGFSRVAAKATPGVVYIENFPKTGNQ²AIASPGNKRGFQ
ENPFDYFNDEFFNRFFGLPSH³REQQRPQQRDAVRGTGFIVSEDGYV
VTNHHVVEDAGKIHVTLHDGQKYTAKIVGLDPKTDLAVIKIQAEKLP
FLT⁴FGNSDQLQIGDWAIAIGNPFGLQATVTVGVVSAKGRNQLHIVD
FEDFIQTDAAINPGNSGGPLLNINGQVIGVNTAIVSGSGGYIGIGFA
IPSLMAKRVIDQLISDGQVTR
[First PDZ protein-protein interaction domain
(residues 300-392)]:
GFLGVTLQPIDSELATCYKLEKVYGALV⁵TDVVKGSPAEKAGLRQED
VIVAYNGKEVESLSALRNAISLMMPGTRVILKIVREGKTIEIPVTVT
[Second conserved PDZ protein-protein interaction
domain (residues 393-497)]:
QIPTEDGVSALQKMGVRVQNITPEICKKLGLAADTRGILVVAVEA⁶G
SPAASAGVAPGQLILAVNRQRVASVEELNQVLKNSKGENVLLMVSQG
DVVRFIVLKSDE
Three regins that contain CD4(+) Th1 cell epitopes can thus be identified:
(1) The solvent-exposed C-terminal region, beginning from Cathepsin site 5. (amino
acids 327-497) (SEQ ID No. 18);
(2) The solvent-exposed C-terminal region, beginning from Cathepsin site 6. (amino
acids 437-497) (SEQ ID No. 19);
(3) The solvent-exposed C-terminal region, between Cathepsin sites 5 and 6. (amino
acids 327-436) (SEQ ID No. 20). (Carmicle, S., et al., J. Biol. Chem., 227(1): 155-
160, 2002)

The C-terminal epitopic fragment from C. trachomatis serovar L₂, SEQ ID NO: 18, has about 99.4% or about 91.3% sequence identity with the corresponding portions of HtrA from C. trachomatis serovars D or MoPn, respectively. Amino acids 437-497 of SEQ ID NO: 2, SEQ ID NO: 19, has about 100% or about 89% sequence identity with the corresponding portions of HtrA from C. trachomatis serovars D or MoPn, respectively. Amino acids 327-436 of SEQ ID NO: 2, SEQ ID NO: 20, has about 94% or about 91% sequence identity with the corresponding portions of HtrA from C. trachomatis serovars D or MoPn, respectively.

The present invention provides an isolated polynucleotide encoding a polypeptide comprising, consisting essentially of, or consisting of an amino acid sequence at least 70%, 75%, 80%, 85%, 90%, 95%, 96%, 97%, 98%, 99%, or 100% identical to amino acids 327-497 of SEQ ID NO: 2, amino acids 437-497 of SEQ ID NO: 2, or amino acids 327-436 of SEQ ID NO: 2, provided that the amino acid sequence is not SEQ ID NO: 6 or SEQ ID NO: 8, wherein the polypeptide when administered to a subject in need thereof in a sufficient amount, induces an immune response against Chlamydia sp. The present invention also includes a polynucleotide comprising, consisting essentially of, or consisting of a nucleic acid sequence at least 60%, 70%, 75%, 80%, 85%, 90%, 95%, 96%, 97%, 98%, 99%, or 100% identical to nucleotide residues 979-1491 of SEQ ID NO: 1, nucleotide residues 1307-1491 of SEQ ID NO: 1, or nucleotide residues 976-1308 of SEQ ID NO: 1, provided that the amino acid sequence encoded by said nucleic acid sequence is not SEQ ID NO: 6, or SEQ ID NO: 8, wherein the polynucleotide encodes the polypeptide that can induce an immune response against Chlamydia sp. when administered to a subject in need thereof in a sufficient amount.

The invention further provides any combinations of fragments produced by Cathepsin S cleavage sites shown in Table 4. For example, the present invention is directed to a polynucleotide encoding a polypeptide comprising, consisting essentially of, or consisting of an amino acid sequence at least 70%, 75%, 80%, 85%, 90%, 95%, 96%, 97%, 98%, 99%, or 100% identical to amino acids 1-19, amino acids 1-79, amino acids 1-112, amino acids 1-187, amino acids 1-326, amino acids 1-436, amino acids 20-79, amino acids 20-112, amino acids 20-187, amino acids 20-326, amino acids 20-436, amino acids 20-497, amino acids 80-112, amino acids 80-187, amino acids 80-326, amino acids 80-436, amino acids 80-497, amino acids 113-187, amino acids 113-326, amino acids 113-436, amino acids 113-497, amino acids 188-326, amino acids 188-436, amino acids 188-497, amino acids 327-436, amino acids 327-497, amino acids 437-497 of SEQ ID NO: 2 or two or more such amino acid sequences in combination, provided that the amino acid sequence is not SEQ ID NOs: 6, or 8, wherein the polypeptide when administered to a subject in need thereof in a sufficient amount, induces an immune response against Chlamydia sp.

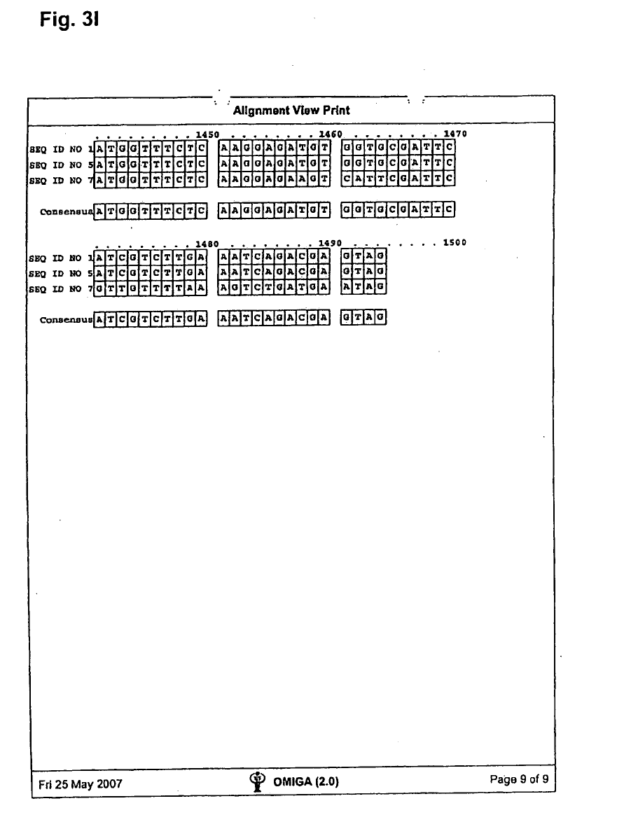

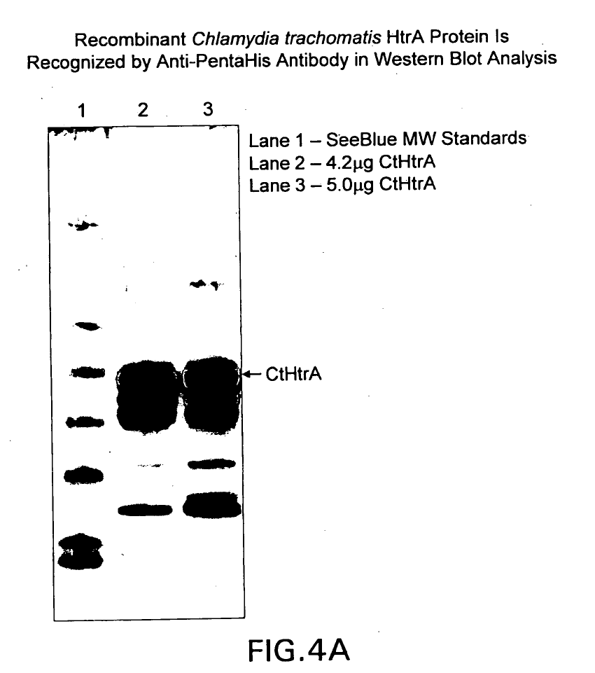

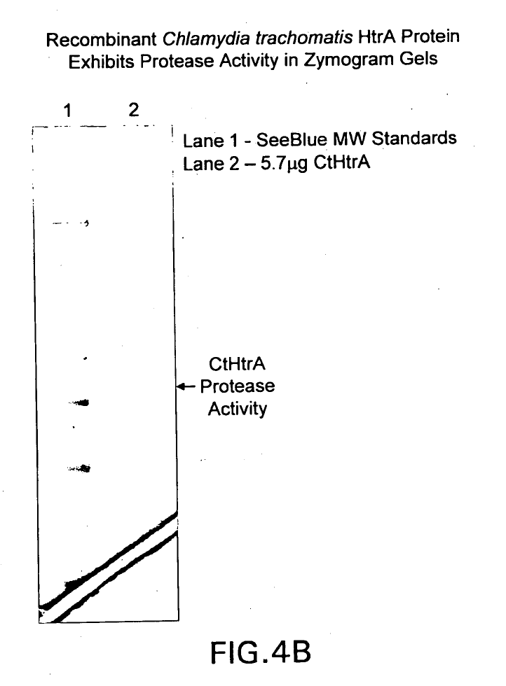

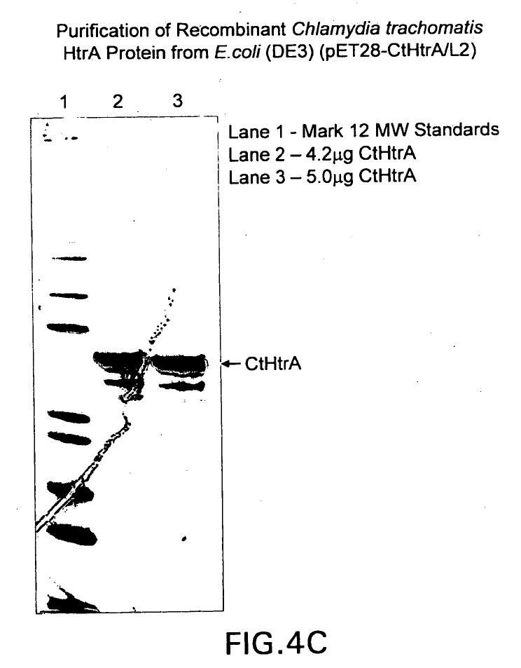

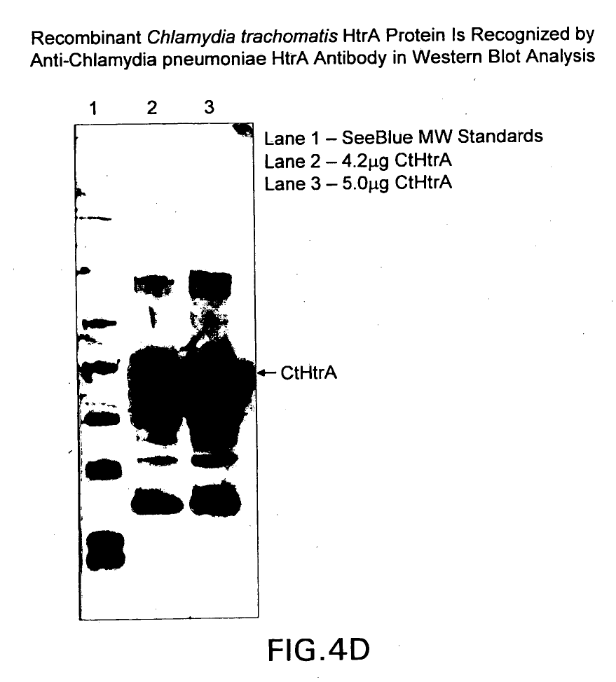

Auto-catalytic fragments of full-length HtrA which comprise B cell epitopes were observed experimentally. HtrA is well known to catalyze auto-digestion. (Krojer, T., et al., Nature, 416:455-459, 2002; Hu, S., et al., J. Biol. Chem., 273(51):34406-12, 1998; Gray, C., et al., Eur. J. Biochem., 267(18):5699-710, 2000; Clausen, T., et al., Mol. Cell., 10(3):443-55, 2002). As shown in FIG. 4(D), in a Western Blot of purified C. trachomatis HtrA immunostained with a polyclonal rabbit anti-serum (K306) raised against purified C. pneumoniae HtrA, several bands are apparent in addition to the principal band. These additional bands are autocatalytic fragments of full-length HtrA and react with antibody K306.

The predominant band in FIG. 4(D), migrating with apparent molecular weight of approximately 52 kD, corresponds to full length mature HtrA, which has a calculated molecular weight 53,209 daltons. Additional, strong immunostaining bands are observed with apparent molecular weights of approximately 47 kD and 40 kD along with faint bands of lower molecular weight, approximately 30 kD and 16 kD. These immunoreactive auto-catalytic fragments of C. trachomatis HtrA shown in Table 5 are quite similar to those observed with human HtrA.

HtrA typically cleaves at Valine-Valine (“VV”), Isoleucine-Valine (“IV”) or Leucine-Valine (“LV”) sites within an interior, solvent protected region. (Kolmar, H., et al., J. Bacteriol., 178(2):5925-5929, 1996; Jones, C., et al., J. Bacteriol., 184(20):5762-5771, 2002). As shown in Tables 5, 6 and 7, the observed apparent molecular weight of the additional immunoreactive bands in FIG. 4(D) corresponds to the expected molecular weight of immunoreactive auto-digestion fragments. HtrA auto-digestion is expected to proceed primarily against the solvent-protected serine protease domain comprising the N-terminal portion of the molecule. The C-terminal portion of the molecule, comprising highly solvent-exposed PDZ protein-protein interaction domains, is expected to contain B-cell epitopes. Table 5 shows auto-cleavage sites in C. trachomatis HtrA. Each site is underlined, bold-faced, and identified by number. Domain boundaries are also identified. Table 7 shows the correlation between observed apparent molecular weight of immunoreactive fragments and the expected molecular weight of an immunoreactive C-terminal fragment from which were used to identify the auto-catalytic fragments. Specific immunoreactive fragments are identified as follows:

SEQ ID NO: 27—amino acids 69-497 of SEQ ID NO: 2; cleavage at HtrA site 1

SEQ ID NO: 28—amino acids 145-497 of SEQ ID NO: 2; cleavage at HtrA site 2b

SEQ ID NO: 29—amino acids 219-497 of SEQ ID NO: 2; cleavage at HtrA site 3

The sequence identity of SEQ ID NOs: 27-29 are described in Table 8.

The present invention includes a polynucleotide encoding a polypeptide comprising, consisting essentially of, or consisting of an amino acid sequence at least 70%, 75%, 80%, 85%, 90%, 95%, 96%, 97%, 98%, 99%, or 100% identical to amino acids 69-497 of SEQ ID NO: 2, amino acids 145-497 of SEQ ID NO: 2, amino acids 219-497 of SEQ ID NO: 2, provided that the amino acid sequence is not SEQ ID NOs: 6 or 8, wherein the polypeptide when administered to a subject in need thereof in a sufficient amount, induces an immune response against Chlamydia sp. The present invention also includes a polynucleotide comprising, consisting essentially of, or consisting of a nucleic acid sequence at least 60%, 70%, 75%, 80%, 85%, 90%, 95%, 96%, 97%, 98%, 99%, or 100% identical to nucleotide residues 205-1491 of SEQ ID NO: 1, nucleotide residues 433-1491 of SEQ ID NO: 1, or nucleotide residues 655-1491 of SEQ ID NO: 1, provided that the amino acid sequence encoded by the nucleotide sequence is not SEQ ID NO: 6 or SEQ ID NO: 8, wherein the polynucleotide encodes a polypeptide that can induce an immune response against Chlamydia sp. when administered to a subject in need thereof in a sufficient amount.

TABLE 5
C. trachomatis HtrA self-cleavage sites

[Serine protease domain (residues 1-299)]:
MVKRLLCVLLSTSVFSSPMLGYSASKKDSKADICLAVSSGDQEVSQ
EDLLKEVSRGFSRVAAKATPGVV¹YIENFPKTGNQAIASPGNKRGF
QENPFDYFNDEFFNRFFGLPSHREQQRPQQRDAVRGTGFIVSEDGYV
V^{2a}TNHHVV^{2b}EDAGKIHVTLHDGQKYTAKIVGLDPKTDLAVIKIQA
EKLPFLTFGNSDQLQIGDWAIAIGNPFGLQATVTVGVV³SAKGRNQL
HIVDFEDFIQTDAAINPGNSGGPLLNINGQVIGVNTAIV⁴SGSGGYI
GIGFAIPSLMAKRVIDQLISDGQVTR
[First PDZ protein-protein interaction domain
(residues 300-392)]:
GFLGVTLQPIDSELATCYKLEKVYGALV^{5a}TDVV^{5b}KGSPAEKAGLR
QEDVIV⁶AYNGKEVESLSALRNAISLMMPGTRVILKIVREGKTIEIP
VTVT
[Second conserved PDZ protein-protein
interaction domain (residues 393-497)]:
QIPTEDGVSALQKMGVRVQNITPEICKKLGLAADTRGILVV⁸RFIV
LKSDE
HtrA self-cleavage sites in C. trachomatis HtrA are individually numbered and underlined in
bold. HtrA has broad specificity, but typically cleaves at VV, IV or LV sites within an interior,
solvent protected region. (Kolmar, et al., J. Bacteriol., 178(2): 5925-5929, 1996; Jones, et al., 1
Bacteriol., 184(20): 5762-5771, 2002).
(a) Site 1: amino acids 1-68; amino acids 69-497 of SEQ ID NO: 2
(b) Site 2a: amino acids 1-138; amino acids 139-497 of SEQ ID NO: 2
(c) Site 2b: amino acids 1-144; amino acids 145-497 of SEQ ID NO: 2
(d) Site 3: amino acids 1-218; amino acids 219-497 of SEQ ID NO: 2
(e) Site 4: amino acids 1-265; amino acids 266-497 of SEQ ID NO: 2
(f) Site 5a: amino acids 1-326; amino acids 327-497 of SEQ ID NO: 2
(g) Site 5b: amino acids 1-330; amino acids 331-497 of SEQ ID NO: 2
(h) Site 6: amino acids 1-347; amino acids 348-497 of SEQ ID NO: 2
(i) Site 7: amino acids 1-431; amino acids 432-497 of SEQ ID NO: 2
(j) Site 8: amino acids 1-487; amino acids 489-497 of SEQ ID NO: 2

TABLE 6
C. trachomatis HtrA N-terminal
self-cleavage fragments

1. Between N-terminus and HtrA site 1, MW 7193
MVKRLLCVLLSTSVFSSPMLGYSASKKDSKADICLAVSSGDQEVSQE
DLLKEVSRGFSRVAAKATPGV
2. Between HtrA sites 1 and 2b, MW 8821
VYIENFPKTGNQAIASPGNKRGFQENPFDYFNDEFFNRFFGLPSHRE
QQRPQQRDAVRGTGFIVSEDGYVVTNHHV
3. Between HtrA sites 2b and 3, MW 7886
VEDAGKIHVTLHDGQKYTAKIVGLDPKTDLAVIKIQAEKLPFLTFGN
SDQLQIGDWAIAIGNPFGLQATVTVGV
4. Between HtrA sites 3 and 5a, molecular weight
11,722
VSAKGRNQLHIVDFEDFIQTDAAINPGNSGGPLLNINGQVIGVNTAI
VSGSGGYI
GIGFAIPSLMAKRVIDQLISDGQVTRGFLGVTLQPIDSELATCYKLE
KVYGALVTV
Shown are N-terminal auto-cleavage fragments of C. trachomatis HtrA

TABLE 7
Correlation between observed and expected apparent molecular weights
of immunoreactive fragments of C. trachomatis HtrA

Apparent
Molecular Weight

| Observed | Expected | Immunoreactive fragment (residues) |
|---|---|---|
| 52 kD | 53 kD | 1-497 (SEQ ID NO: 2) |
| 47 kD | 46 kD | 69-497; cleavage at HtrA site 1 |
| | | (SEQ ID NO: 27) |
| 40 kD | 37 kD | 145-497; cleavage at HtrA site 2b |
| | | (SEQ ID NO: 28) |
| 30 kD | 29 kD | 219-497; cleavage at HtrA site 3 |
| | | (SEQ ID NO: 29) |
| 18 kD | 16 kD | 327-497; cleavage at HtrA site 5a |
| | | (SEQ ID NO: 30) |

Shown is the correlation between observed apparent molecular weight of immunoreactive fragments of C. trachomatis HtrA and the expected molecular weight of an immunoreactive C-terminal fragment from which HtrA auto-cleavage fragments have been removed from the N-terminal region.

While the invention has been described in conjunction with specific embodiments, it is evident that many alternatives, modifications and variations will be apparent to a person of ordinary skill in the art in light of the foregoing description. Accordingly, the present invention embraces all such alternatives, modifications and variations that fall within the spirit and scope of the appended claims. Table 8 shows some non-limiting examples of such variations of the fragments and their sequence identity to SEQ ID NO: 2.

TABLE 8
Fragments and Variations of C. trachomatis HtrA Polypeptide

| | SEQ ID | SEQ ID | SEQ ID |
|---|---|---|---|
| Sequences | NO: 2 | NO: 6 | NO: 8 |
| amino acids 1-19 (SEQ ID NO: 21) | 100% | 98% | 94% |
| amino acids 1-68 | 100% | 98% | 82% |
| amino acids 1-79 (SEQ ID NO: 22) | 100% | 99% | 83% |
| amino acids 1-112 (SEQ ID NO: 23) | 100% | 99% | 88% |
| amino acids 1-144 | 100% | 99% | 89% |
| amino acids 1-218 | 100% | 99% | 91% |
| amino acids 1-299 | 100% | 99% | 93% |
| amino acids 1-326 | 100% | 99% | 93% |
| amino acids 1-392 | 100% | 99% | 93% |
| amino acids 1-436 | 100% | 99% | 93% |
| amino acids 1-449 | 100% | 99% | 93% |
| amino acids 17-68 | 100% | 100% | 78% |
| amino acids 17-79 | 100% | 100% | 80% |
| amino acids 17-112 | 100% | 100% | 87% |
| amino acids 17-144 | 100% | 100% | 89% |
| amino acids 17-218 | 100% | 100% | 91% |
| amino acids 17-299 | 100% | 99% | 93% |
| amino acids 17-326 | 100% | 99% | 93% |
| amino acids 17-392 | 100% | 99% | 93% |
| amino acids 17-436 | 100% | 99% | 93% |
| amino acids 17-449 | 100% | 99% | 93% |
| amino acids 20-68 | 100% | 100% | 77% |
| amino acids 20-79 (SEQ ID NO: 24) | 100% | 100% | 80% |
| amino acids 20-112 (SEQ ID NO: 25) | 100% | 100% | 87% |
| amino acids 20-144 | 100% | 100% | 89% |
| amino acids 20-218 | 100% | 99% | 91% |
| amino acids 20-299 | 100% | 99% | 93% |
| amino acids 20-326 | 100% | 99% | 93% |
| amino acids 20-392 | 100% | 99% | 93% |
| amino acids 20-436 | 100% | 99% | 93% |
| amino acids 20-449 | 100% | 99% | 93% |
| amino acids 20-497 | 100% | 99% | 92% |
| amino acids 69-112 | 100% | 100% | 97% |
| amino acids 69-144 | 100% | 100% | 97% |
| amino acids 69-218 | 100% | 99% | 95% |
| amino acids 69-299 | 100% | 99% | 96% |
| amino acids 69-326 | 100% | 99% | 96% |
| amino acids 69-392 | 100% | 99% | 95% |
| amino acids 69-436 | 100% | 99% | 95% |
| amino acids 69-449 | 100% | 99% | 95% |
| amino acids 69-497 (SEQ ID NO: 27) | 100% | 99% | 94% |
| amino acids 80-112 (SEQ ID NO: 26) | 100% | 100% | 100% |
| amino acids 80-144 | 100% | 100% | 98% |
| amino acids 80-218 | 100% | 99% | 95% |
| amino acids 80-299 | 100% | 99% | 96% |
| amino acids 80-326 | 100% | 99% | 96% |
| amino acids 80-392 | 100% | 99% | 96% |
| amino acids 80-436 | 100% | 99% | 95% |
| amino acids 80-449 | 100% | 99% | 95% |
| amino acids 80-497 | 100% | 99% | 94% |
| amino acids 113-144 | 100% | 100% | 96% |
| amino acids 113-218 | 100% | 100% | 95% |
| amino acids 113-299 | 100% | 99% | 96% |
| amino acids 113-326 | 100% | 99% | 96% |
| amino acids 113-392 | 100% | 99% | 95% |
| amino acids 113-436 | 100% | 99% | 95% |
| amino acids 113-449 | 100% | 99% | 95% |
| amino acids 113-497 | 100% | 99% | 94% |
| amino acids 145-218 | 100% | 98% | 93% |
| amino acids 145-299 | 100% | 99% | 96% |
| amino acids 145-326 | 100% | 99% | 96% |
| amino acids 145-392 | 100% | 99% | 95% |
| amino acids 145-436 | 100% | 99% | 95% |
| amino acids 145-449 | 100% | 99% | 95% |
| amino acids 145-497 (SEQ ID NO: 28) | 100% | 99% | 93% |
| amino acids 219-299 | 100% | 98% | 97% |
| amino acids 219-326 | 100% | 98% | 98% |
| amino acids 219-392 | 100% | 98% | 95% |
| amino acids 219-436 | 100% | 99% | 95% |
| amino acids 219-449 | 100% | 99% | 95% |
| amino acids 219-497 (SEQ ID NO: 29) | 100% | 99% | 91% |
| amino acids 300-392 | 100% | 98% | 94% |
| amino acids 300-436 | 100% | 99% | 93% |
| amino acids 300-449 | 100% | 99% | 93% |
| amino acids 300-497 | 100% | 99% | 91% |
| amino acids 327-392 | 100% | 98% | 93% |
| amino acids 327-436 (SEQ ID NO: 20) | 100% | 94% | 91% |
| amino acids 327-449 | 100% | 99% | 93% |
| amino acids 327-497 (SEQ ID NO: 18) | 100% | 99% | 91% |
| amino acids 393-436 | 100% | 100% | 92% |
| amino acids 393-449 | 100% | 100% | 92% |
| amino acids 393-497 | 100% | 100% | 89% |
| amino acids 437-497 | 100% | 100% | 89% |
| amino acids 450-497 (SEQ ID NO: 19) | 100% | 100% | 89% |

In some embodiments, the present invention is directed to a polynucleotide which encodes a polypeptide comprising, consisting essentially of, or consisting of an amino acid sequence at least 70%, 75%, 80%, 85%, 90%, 95%, 96%, 97%, 98%, 99%, or 100% identical to a reference amino acid sequence selected from a group consisting of amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ED NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 30; and a combination of at least two of any of said polypeptide fragments or variants or derivatives thereof, provided that said amino acid sequence is not SEQ ID NOs: 6 or 8, wherein said polypeptide when administered to a subject in need thereof in a sufficient amount, induces an immune response against Chlamydia sp. The polynucleotide of the present invention can contain any polynucleotide encoding a polypeptide comprising, consisting essentially of, or consisting of an amino acid sequence at least 70%, 75%, 80%, 85%, 90%, 95%, 96%, 97%, 98%, 99%, or 100% identical to any combinations of fragments producible by the Cathepsin S cleavage sites in Table 4 and/or auto-cleavage sites of C. trachomatis HtrA shown in Table 5.

The present invention further includes a polynucleotide comprising, consisting essentially of, or consisting of a nucleic acid sequence at least 60%, 70%, 75%, 80%, 85%, 90%, 95%, 96%, 97%, 98%, 99%, or 100% identical to the corresponding nucleotide residues of SEQ ID NO: 1 encoding any fragments disclosed in Tables 3-8, or variants or derivatives thereof, provided that the amino acid sequence encoded by the nucleic acid sequence is not SEQ ID NOs: 6 or 8, wherein the polypeptide encoded by said nucleic acid sequence induces an immune response against Chlamydia sp. when administered to a subject in need thereof in a sufficient amount. A person of ordinary skill in the art can readily calculate the exact coordinate of the nucleotide residues of SEQ ID NO: 1 encoding fragments of SEQ ID NO: 2.

In some embodiments, the present invention is further directed to a polynucleotide which encodes a polypeptide comprising, consisting essentially of, or consisting of an amino acid sequence selected from a group consisting of amino acids 1-19 of SEQ ID NOs:2, 6, or 8; amino acids 1-68 of SEQ ID NOs:2, 6, or 8, amino acids 1-79 of SEQ ID NOs:2, 6, or 8; amino acids 1-112 of SEQ ID NOs:2, 6, or 8; amino acids 1-144 of SEQ ID NOs:2, 6, or 8; amino acids 1-218 of SEQ ID NOs:2, 6, or 8; amino acids 1-299 of SEQ ID NOs:2, 6, or 8; amino acids 1-326 of SEQ ID NOs:2, 6, or 8; amino acids 1-392 of SEQ ID NOs:2, 6, or 8; amino acids 1-436 of SEQ ID NOs:2, 6, or 8; amino acids 1-449 of SEQ ID NOs:2, 6, or 8; amino acids 17-68 of SEQ ID NOs:2, 6, or 8; amino acids 17-79 of SEQ ID NOs:2, 6, or 8; amino acids 17-112 of SEQ ID NOs:2, 6, or 8; amino acids 17-144 of SEQ ID NOs:2, 6, or 8; amino acids 17-218 of SEQ ID NOs:2, 6, or 8; amino acids 17-299 of SEQ ID NOs:2, 6, or 8; amino acids 17-326 of SEQ ID NOs:2, 6, or 8; amino acids 17-392 of SEQ ID NOs:2, 6, or 8; amino acids 17-436 of SEQ ID NOs:2, 6, or 8; amino acids 17-449 of SEQ ID NOs:2, 6, or 8; amino acids 20-68 of SEQ ID NOs:2, 6, or 8; amino acids 20-79 of SEQ ID NOs:2, 6, or 8; amino acids 20-112 of SEQ ID NOs:2, 6, or 8; amino acids 20-144 of SEQ ID NOs:2, 6, or 8; amino acids 20-218 of SEQ ID NOs:2, 6, or 8; amino acids 20-299 of SEQ ID NOs:2, 6, or 8; amino acids 20-326 of SEQ ID NOs:2, 6, or 8; amino acids 20-392 of SEQ ID NOs:2, 6, or 8; amino acids 20-436 of SEQ ID NOs:2, 6, or 8; amino acids 20-449 of SEQ ID NOs:2, 6, or 8; amino acids 20-497 of SEQ ID NOs:2, 6, or 8; amino acids 69-112 of SEQ ID NOs:2, 6, or 8; amino acids 69-144 of SEQ ID NOs:2, 6, or 8; amino acids 69-218 of SEQ ID NOs:2, 6, or 8; amino acids 69-299 of SEQ ID NOs:2, 6, or 8; amino acids 69-326 of SEQ ID NOs:2, 6, or 8; amino acids 69-392 of SEQ ID NOs:2, 6, or 8; amino acids 69-436 of SEQ ID NOs:2, 6, or 8; amino acids 69-449 of SEQ ID NOs:2, 6, or 8; amino acids 69-497 of SEQ ID NOs:2, 6, or 8; amino acids 80-112 of SEQ ID NOs:2, 6, or 8; amino acids 80-144 of SEQ ID NOs:2, 6, or 8; amino acids 80-218 of SEQ ID NOs:2, 6, or 8; amino acids 80-299 of SEQ ID NOs:2, 6, or 8; amino acids 80-326 of SEQ ID NOs:2, 6, or 8; amino acids 80-392 of SEQ ID NOs:2, 6, or 8; amino acids 80-436 of SEQ ID NOs:2, 6, or 8; amino acids 80-449 of SEQ ID NOs:2, 6, or 8; amino acids 80-497 of SEQ ID NOs:2, 6, or 8; amino acids 113-144 of SEQ ID NOs:2, 6, or 8; amino acids 113-218 of SEQ ID NOs:2, 6, or 8; amino acids 113-299 of SEQ ID NOs:2, 6, or 8; amino acids 113-326 of SEQ ID NOs:2, 6, or 8; amino acids 113-392 of SEQ ID NOs:2, 6, or 8; amino acids 113-436 of SEQ ID NOs:2, 6, or 8; amino acids 113-449 of SEQ ID NOs:2, 6, or 8; amino acids 113-497 of SEQ ID NOs:2, 6, or 8; amino acids 145-218 of SEQ ID NOs:2, 6, or 8; amino acids 145-299 of SEQ ID NOs:2, 6, or 8; amino acids 145-326 of SEQ ID NOs:2, 6, or 8; amino acids 145-392 of SEQ ID NOs:2, 6, or 8; amino acids 145-436 of SEQ ID NOs:2, 6, or 8; amino acids 145-449 of SEQ ID NOs:2, 6, or 8; amino acids 145-497 of SEQ ID NOs:2, 6, or 8; amino acids 219-299 of SEQ ID NOs:2, 6, or 8; amino acids 219-326 of SEQ ID NOs:2, 6, or 8; amino acids 219-392 of SEQ ID NOs:2, 6, or 8; amino acids 219-436 of SEQ ID NOs:2, 6, or 8; amino acids 219-449 of SEQ ID NOs:2, 6, or 8; amino acids 219-497 of SEQ ID NOs:2, 6, or 8; amino acids 300-392 of SEQ ID NOs:2, 6, or 8; amino acids 300-436 of SEQ ID NOs:2, 6, or 8; amino acids 300-449 of SEQ ID NOs:2, 6, or 8; amino acids 300-497 of SEQ ID NOs:2, 6, or 8; amino acids 327-392 of SEQ ID NOs:2, 6, or 8; amino acids 327-436 of SEQ ID NOs:2, 6, or 8; amino acids 327-449 of SEQ ID NOs:2, 6, or 8; amino acids 327-497 of SEQ ID NOs:2, 6, or 8; amino acids 393-436 of SEQ ID NOs:2, 6, or 8; amino acids 393-449 of SEQ ID NOs:2, 6, or 8; amino acids 393-497 of SEQ ID NOs:2, 6, or 8; amino acids 437-497 of SEQ ID NOs:2, 6, or 8; amino acids 450-497 of SEQ ID NOs:2, 6, or 8; and a combination of at least two of any of said polypeptide fragments or variants or derivatives thereof, provided that the amino acid sequence is not SEQ ID NOs: 6 or 8, wherein said polypeptide when administered to a subject in need thereof in a sufficient amount, induces an immune response against Chlamydia sp.

Polynucleotides or nucleic acid sequences defined herein are represented by one-letter symbols for the bases as follows: A (adenine) C (cytosine) G (guanine) T (thymine) U (uracil) M (A or C) R (A or G) W (A or T/U); S (C or G); Y (C or T/U); K (G or T/U); V (A or C or G; not T/U); H (A or C or T/U; not G); D (A or G or T/U; not C); B (C or G or T/U; not A); N (A or C or G or T/U) or (unknown).

In some embodiments of the present invention the polynucleotide is isolated. As used herein, the term “isolated” means that the polynucleotide or polypeptide or fragment, variant, or derivative thereof has been removed from other biological materials with which it is naturally associated. An example of an isolated polynucleotide is a recombinant polynucleotide contained in a vector. Further examples of an isolated polynucleotide include recombinant polynucleotides maintained in heterologous host cells or purified (partially or substantially) polynucleotides in solution. Isolated RNA molecules include in vivo or in vitro RNA transcripts of the polynucleotides of the present invention. Isolated polynucleotides or nucleic acids according to the present invention further include such molecules produced synthetically.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the invention. For example, a purified polypeptide of the present invention includes a polypeptide that is at least 70-100% pure, i.e., a polypeptide which is present in a composition wherein the polypeptide constitutes 70-100% by weight of the total composition. In some embodiments, the purified polypeptide of the present invention is 75%-99% by weight pure, 80%-99% by weight pure, 90-99% by weight pure, or 95% to 99% by weight pure. An example of an isolated polynucleotide is a recombinant polynucleotide contained in a vector. Further examples of an isolated polynucleotide include recombinant polynucleotides maintained in heterologous host cells or purified (partially or substantially) polynucleotides in solution. Isolated RNA molecules include in vivo or in vitro RNA transcripts of the polynucleotides of the present invention. Isolated polynucleotides or nucleic acids according to the present invention further include such molecules produced synthetically. The relative degree of purity of a polynucleotide or polypeptide of the invention is easily determined by well-known methods.

Further included is an isolated polynucleotide comprising a nucleic acid encoding an amino acid sequence at least 70%, 75%, 80%, 85%, 90%, 95%, 96%, 97%, 98%, 99%, or 100% identical to SEQ ID NO: 2, wherein the amino acid sequence has a reduced or decreased catalytic activity. The term “catalytic activity” described herein is defined as a proteolytic activity, i.e. ability to degrade membrane or periplasmic proteins, as well as ability to degrade itself. The substrate for the proteolytic activity can be any proteins found in an organism, membrane-associated or periplasmic proteins, and includes autocatalytic activity. The term “a reduced or decreased catalytic activity” further includes, for instance, C. trachomatis or C. pneumoniae HtrA polypeptides that exhibit a reduced or decreased substrate binding compared to its naturally-occurring polypeptide. Such polynucleotides encoding a C. trachomatis or C. pneumoniae HtrA polypeptide, or fragments, analogs, derivatives, or variants thereof with a reduced or decreased catalytic activity can be readily predicted or identified by a person of ordinary skill in the art using routine assays, as shown in FIG. 4(B). The polynucleotides with an ablated or reduced protease activity can be readily produced by an ordinary skilled person in the art using conventional technologies, e.g., site directed mutagenesis. For example, one or more amino acids in the catalytic domain, as shown in Table 3, can be substituted, replaced, modified, or deleted to ablate or reduce the catalytic activity of the HtrA polypeptide while the polypeptide retains immunogenicity or antigenicity.

In one embodiment, the amino acid sequence of SEQ ID NOs: 2, 4, 6, or 8, variants, derivatives, analogs, or fragments thereof, encoded by the polynucleotide of the present invention, can be substituted, replaced, modified, or deleted with one or more of amino acids at one or more amino acid residues selected from the group consisting of residue 105 of SEQ ID NOs: 2, 4, 6, or 8; residue 137 of SEQ ID NOs: 2, 4, 6, or 8, residue 143 of SEQ ID NOs: 2, 4, 6, or 8, residue 155 of SEQ ID NOs: 2, 4, 6, or 8; and residue 247 of SEQ ID NOs: 2, 4, 6, or 8. The polypeptide comprising, consisting essentially of, or consisting of the amino acid sequence retains immunogenicity or antigenicity after the substitution, replacement, modification, or deletion.

Technologies that are used to construct protease ablated/reduced HtrA polypeptide is readily available in the art. For example, it is well known in the art that many trypsin-like serine proteases contain a consensus sequence Gly-Asp-Ser-Gly-Gly-Pro-Lys. Johnson, et al., Mol. Microbiol. 5:410-7 (1991). Likewise, a similar consensus sequence, Gly-Asn-Ser-Gly-Gly, is found at amino acids 208-212 of the E. coli full-length HtrA polypeptide. (Skorko-Glonek, et al., J. Biol. Chem. 270: 11140-11146 (1995)), incorporated herein by reference in its entirety. The particular consensus sequence is known to play an important role in the catalytic activity of the HtrA polypeptides. In particular, the E. coli HtrA polypeptide variant in which serine at residue 210 is substituted with alanine shows complete lack of catalytic activity. Id. A similar mutant in which histidine at residue 105 was substituted with arginine also showed reduced catalytic activity. Id. Therefore, a person of ordinary skill in the art can, for example, calculate the residues in the C. trachomatis HtrA polypeptide corresponding to S²¹⁰ or H¹⁰⁵ of the E. coli HtrA mature polypeptides. The same consensus sequence found in the E. coli HtrA polypeptide is found in the C. trachomatis HtrA polypeptides at amino acids 245-249 of SEQ ID NO: 2 for C. trachomatis serotype L₂. The corresponding residues in SEQ ID NOs: 2, 4, 6, and 8 to S²¹⁰ and H¹⁰⁵ in E. coli HtrA are S²⁴⁷ and H¹⁴³.

The present invention thus includes a polynucleotide which encodes a C. trachomatis polypeptide, fragment, derivative, analog, or variant thereof, which has an amino acid at 247 or 143 of SEQ ID NOs: 2, 6, or 8 other than serine or histidine, respectively. For example, the amino acid at 247 of SEQ ID NOs: 2, 4, 6, or 8 or any fragments, derivatives, analogs, or variants thereof can be alanine, and the amino acid at 143 of SEQ ID NOs: 2, 4, 6, or 8 or any fragments, derivatives, analogs, or variants thereof can be arginine. Similarly, the present invention includes a C. trachomatis polynucleotide which encodes an HtrA polypeptide, which may be resistant to auto-cleavage activity. A person of ordinary skill in the art can readily make such modifications or substitutions to make the polypeptides with or without a specific activity. One skilled in the art can test the protease activity as shown in FIG. 4(B).

The present invention also includes a polynucleotide comprising, consisting essentially of, or consisting of a nucleic acid sequence encoding an amino acid sequence at least 70%, 75%, 80%, 85%, 90%, 95%, 96%, 97%, 98%, 99%, or 100% identical to the HtrA polypeptide sequence or fragment thereof without a part of or whole catalytic domain, wherein the polynucleotide has no or reduced catalytic activity. The catalytic domain, shown in Table 3, can have one or more amino acid deletions that give rise to no catalytic activity or a reduced activity.

In some embodiments, the present invention is directed to a polynucleotide further comprising a heterologous nucleic acid. The heterologous nucleic acid can, in some embodiments, encode a heterologous polypeptide fused to the polypeptide. The term “heterologous” refers to any additional biological components that are not identical with the subject biological component. The components may be host cells, genes, or regulatory regions, such as promoters. The heterologous components can function together, as when a promoter heterologous to a gene is operably linked to the gene. Another example is where a chlamydial sequence is heterologous to a mouse host cell. A further example would be two epitopes from the same or different proteins which have been assembled in a single protein. An example includes a polynucleotide which encodes an Fc portion of an antibody linked to a fragment of a C. trachomatis HtrA polypeptide of the invention. Another example is a full-length or mature HtrA polypeptide fused to a 6 histidine tag, i.e., SEQ ID NO: 4. The present invention includes a polynucleotide comprising a nucleic acid which encodes an amino acid sequence at least 70%, 75%, 80%, 85%, 90%, 95%, 99%, or 100% identical to SEQ ID NO: 2 without a signal sequence and a heterologous polypeptide, e.g., SEQ ID NO: 4, wherein said polypeptide when administered to a subject in need thereof in a sufficient amount, induces an immune response against Chlamydia sp.

Various heterologous nucleic acids can be used to encode their respective heterologous polypeptides. In some embodiments, the heterologous polypeptide is fused to the polypeptide of the present invention. Various heterologous polypeptides can be used, and can be selected from the group consisting of an N- or C-terminal peptide imparting stabilization, secretion, or simplified purification, i.e., His-tag (SEQ ID NO: 4), ubiquitin tag, NusA tag, chitin binding domain, ompT, ompA, pelB, DsbA, DsbC, c-myc, KSI, polyaspartic acid, (Ala-Trp-Trp-Pro)n (SEQ ID NO:10), polyphenyalanine, polycysteine, polyarginine, B-tag, HSB-tag, green fluorescent protein (GFP), hemagglutinin influenza virus (HAI), calmodulin binding protein (CBP), galactose-binding protein, maltose binding protein (MBP), cellulose binding domains (CBD's), dihydrofolate reductase (DHFR), glutathione-S-transferase (GST), streptococcal protein G, staphylococcal protein A, T7gene10, avidin/streptavidin/Strep-tag, trpE, chloramphenicol acetyltransferase, lacZ (β-Galactosidase), His-patch thioredoxin, thioredoxin, FLAG™ peptide (Sigma-Aldrich), S-tag, and T7-tag. See e.g., Stevens, R. C., Structure, 8:R177-R185 (2000). The heterologous polypeptides can further include any pre- and/or pro-sequences that facilitate the transport, translocations, processing and/or purification of C. trachomatis HtrA vaccine antigens from a host cell or any useful immunogenic sequence, including but not limited to sequences that encode a T-cell epitope of a microbial pathogen, or other immunogenic proteins and/or epitopes.

Other suitable heterologous polypeptides may include other Chlamydia proteins (either native proteins or variants, fragments, or derivatives thereof, e.g., MOMP, PorB, Pmp6, Pmp8, Pmp11, Pmp20, Pmp21, PmpD, PmpE, PmpG, PmpH, PmpI, OmpH, Omp4, Omp5, Omp85, MTP, OmcA, and OmcB), and in some embodiments, a leader sequence capable of directing secretion of translated protein into the periplasmic space or extracellular medium. In some embodiments, the polypeptide of the present invention can exist as a homopolymer, comprising multiple copies of the same polypeptide.

### Codon Optimization

Also included within the scope of the invention are genetically engineered polynucleotides encoding C. trachomatis HtrA variants. Modifications of nucleic acids encoding HtrA polypeptides can readily be accomplished by those skilled in the art, for example, by oligonucleotide-directed site-specific mutagenesis of a polynucleotide coding for an HtrA polypeptide. Such modified polypeptide can be encoded by a codon optimized nucleotide sequence. Such modifications impart one or more amino acid substitutions, insertions, deletions, and/or modifications to expressed HtrA polypeptides including fragments, variants, and derivatives. Such modifications may enhance the immunogenicity of HtrA antigens, for example, by increasing cellular immune responses compared with unmodified polypeptides. Such modification may enhance solubility of the polypeptides. Alternatively, such modifications may have no effect. For example, C. trachomatis HtrA may be modified by introduction, deletion or modification of particular cleavage sites for proteolytic enzymes active in antigen presenting cells, to enhance immune responses to particular epitopes.

In some embodiments, the present invention is directed to a polynucleotide comprising a nucleic acid fragment, which encodes the HtrA polypeptide from Chlamydia trachomatis, e.g., SEQ ID NO:2, SEQ ID NO: 4, SEQ ID NO: 6, or SEQ ID NO: 8 or a fragment, variant, or derivative thereof. As appreciated by one of ordinary skill in the art, various nucleic acid coding regions will encode the same polypeptide due to the redundancy of the genetic code. Deviations in the nucleotide sequence that comprise the codons encoding the amino acids of any polypeptide chain allow for variations in the sequence coding for the gene. Since each codon consists of three nucleotides, and the nucleotides comprising DNA are restricted to four specific bases, there are 64 possible combinations of nucleotides, 61 of which encode amino acids (the remaining three codons encode signals ending translation). The “genetic code” which shows which codons encode which amino acids is reproduced herein as Table 9. As a result, many amino acids are designated by more than one codon. For example, the amino acids alanine and proline are coded for by four triplets, serine and arginine by six, whereas tryptophan and methionine are coded by just one triplet. This degeneracy allows for DNA base composition to vary over a wide range without altering the amino acid sequence of the polypeptides encoded by the DNA.

TABLE 9
The Standard Genetic Code

| | T | C | A | G |
|---|---|---|---|---|
| T | TTT Phe (F) | TCT Ser (S) | TAT Tyr (Y) | TGT Cys (C) |
| | TTC Phe (F) | TCC Ser (S) | TAC Tyr (Y) | TGC |
| | TTA Leu (L) | TCA Ser (S) | TAA Ter | TGA Ter |
| | TTG Leu (L) | TCG Ser (S) | TAG Ter | TGG Trp (W) |
| C | CTT Leu (L) | CCT Pro (P) | CAT His (H) | CGT Arg (R) |
| | CTC Leu (L) | CCC Pro (P) | CAC His (H) | CGC Arg (R) |
| | CTA Leu (L) | CCA Pro (P) | CAA Gln (Q) | CGA Arg (R) |
| | CTG Leu (L) | CCG Pro (P) | CAG Gln (Q) | CGG Arg (R) |
| A | ATT Ile (I) | ACT Thr (T) | AAT Asn (N) | AGT Ser (S) |
| | ATC Ile (I) | ACC Thr (T) | AAC Asn (N) | AGC Ser (S) |
| | ATA Ile (I) | ACA Thr (T) | AAA Lys (K) | AGA Arg (R) |
| | ATG Met (M) | ACG Thr (T) | AAG Lys (K) | AGG Arg (R) |
| G | GTT Val (V) | GCT Ala (A) | GAT Asp (D) | GGT Gly (G) |
| | GTC Val (V) | GCC Ala (A) | GAC Asp (D) | GGC Gly (G) |
| | GTA Val (V) | GCA Ala (A) | GAA Glu (E) | GGA Gly (G) |
| | GTG Val (V) | GCG Ala (A) | GAG Glu (E) | GGG Gly (G) |

It is to be appreciated that any polynucleotide that encodes a polypeptide in accordance with the invention falls within the scope of this invention, irregardless of the codons used.

Many organisms display a bias for use of particular codons to code for insertion of a particular amino acid in a growing polypeptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

The present invention relates to a polynucleotide comprising, consisting essentially of, or consisting of a coding optimized coding region which encodes a polypeptide disclosed herein. For example, a polynucleotide of the present invention comprises, consisting essentially of, or consisting of an amino acid sequence at least 70%, 75%, 80%, 85%, 90%, 95%, 96%, 97%, 98%, 99%, or 100% identical to the amino acid sequences described in Tables 3-8, provided that said amino acid sequence is not SEQ ID NOs: 6, or 8, wherein said polypeptide when administered to a subject in need thereof in a sufficient amount, induces an immune response against Chlamydia sp. The present invention further relates to any polynucleotide fragments or full length HtrA described herein which is encoded fully or partly by a codon-optimized coding region. The codon usage is adapted for optimized expression in the cells of a given prokaryote or eukaryote.

The polynucleotides are prepared by incorporating codons preferred for use in the genes of a given species into the DNA sequence. Also provided are polynucleotide expression constructs, vectors, host cells comprising nucleic acid fragments of codon-optimized coding regions which encode C. trachomatis polypeptides, and various methods of using the polynucleotide expression constructs, vectors, host cells to treat or prevent Chlamydia infections in an animal.

Given the large number of gene sequences available for a wide variety of animal, plant and microbial species, it is possible to calculate the relative frequencies of codon usage. Codon usage tables are readily available, for example, at the “Codon Usage Database” available at http://www.kazusa.or.jp/codon/ (visited May 30, 2006), and these tables can be adapted in a number of ways. See Nakamura, Y., et al., “Codon usage tabulated from the international DNA sequence databases: status for the year 2000” Nucl. Acids Res. 28:292 (2000). Codon usage tables for humans, Escherichia coli, and P. fluerescens calculated from GenBank Release 151.0, are reproduced below as Tables 10-12 (from http://www.kazusa.or.jp/codon/ supra). These tables use mRNA nomenclature, and so instead of thymine (T) which is found in DNA, the tables use uracil (U) which is found in RNA. The tables have been adapted so that frequencies are calculated for each amino acid, rather than for all 64 codons.

TABLE 10
Codon Usage Table for Human Genes (Homo sapiens)

| Amino Acid | Codon | Frequency of Usage |
|---|---|---|
| Phe | UUU | 0.4525 |
| | UUC | 0.5475 |
| Leu | UUA | 0.0728 |
| | UUG | 0.1266 |
| | CUU | 0.1287 |
| | CUC | 0.1956 |
| | CUA | 0.0700 |
| | CUG | 0.4062 |
| Ile | AUU | 0.3554 |
| | AUC | 0.4850 |
| | AUA | 0.1596 |
| Met | AUG | 1.0000 |
| Val | GUU | 0.1773 |
| | GUC | 0.2380 |
| | GUA | 0.1137 |
| | GUG | 0.4710 |
| Ser | UCU | 0.1840 |
| | UCC | 0.2191 |
| | UCA | 0.1472 |
| | UCG | 0.0565 |
| | AGU | 0.1499 |
| | AGC | 0.2433 |
| Pro | CCU | 0.2834 |
| | CCC | 0.3281 |
| | CCA | 0.2736 |
| | CCG | 0.1149 |
| Thr | ACU | 0.2419 |
| | ACC | 0.3624 |
| | ACA | 0.2787 |
| | ACG | 0.1171 |
| Ala | GCU | 0.2637 |
| | GCC | 0.4037 |
| | GCA | 0.2255 |
| | GCG | 0.1071 |
| Tyr | UAU | 0.4347 |
| | UAC | 0.5653 |
| His | CAU | 0.4113 |
| | CAC | 0.5887 |
| Gln | CAA | 0.2541 |
| | CAG | 0.7459 |
| Asn | AAU | 0.4614 |
| | AAC | 0.5386 |
| Lys | AAA | 0.4212 |
| | AAG | 0.5788 |
| Asp | GAU | 0.4613 |
| | GAC | 0.5387 |
| Glu | GAA | 0.4161 |
| | GAG | 0.5839 |
| Cys | UGU | 0.4468 |
| | UGC | 0.5532 |
| Trp | UGG | 1.0000 |
| Arg | CGU | 0.0830 |
| | CGC | 0.1927 |
| | CGA | 0.1120 |
| | CGG | 0.2092 |
| | AGA | 0.2021 |
| | AGG | 0.2011 |
| Gly | GGU | 0.1632 |
| | GGC | 0.3438 |
| | GGA | 0.2459 |
| | GGG | 0.2471 |

TABLE 11
Codon Usage Table for Escherichia Coli.

| Amino Acid | Codon | Frequency of usage |
|---|---|---|
| Phe | UUU | 0.51 |
| | UUC | 0.49 |
| Leu | UUA | 0.11 |
| | UUG | 0.11 |
| | CUU | 0.10 |
| | CUC | 0.10 |
| | CUA | 0.03 |
| | CUG | 0.55 |
| Ile | AUU | 0.47 |
| | AUC | 0.46 |
| | AUA | 0.07 |
| Met | AUG | 1.00 |
| Val | GUU | 0.29 |
| | GUC | 0.20 |
| | GUA | 0.17 |
| | GUG | 0.34 |
| Ser | UCU | 0.19 |
| | UCC | 0.17 |
| | UCA | 0.12 |
| | UCG | 0.13 |
| | AGU | 0.13 |
| | AGC | 0.27 |
| Pro | CCU | 0.16 |
| | CCC | 0.10 |
| | CCA | 0.20 |
| | CCG | 0.55 |
| Thr | ACU | 0.21 |
| | ACC | 0.43 |
| | ACA | 0.30 |
| | ACG | 0.23 |
| Ala | GCU | 0.19 |
| | GCC | 0.25 |
| | GCA | 0.22 |
| | GCG | 0.34 |
| Tyr | UAU | 0.53 |
| | UAC | 0.47 |
| His | CAU | 0.52 |
| | CAC | 0.48 |
| Gln | CAA | 0.31 |
| | CAG | 0.69 |
| Asn | AAU | 0.39 |
| | AAC | 0.61 |
| Lys | AAA | 0.76 |
| | AAG | 0.24 |
| Asp | GAU | 0.59 |
| | GAC | 0.41 |
| Glu | GAA | 0.70 |
| | GAG | 0.30 |
| Cys | UGU | 0.40 |
| | UGC | 0.60 |
| Trp | UGG | 1.00 |
| Arg | CGU | 0.42 |
| | CGC | 0.37 |
| | CGA | 0.05 |
| | CGG | 0.08 |
| | AGA | 0.04 |
| | AGG | 0.03 |
| Gly | GGU | 0.38 |
| | GGC | 0.40 |
| | GGA | 0.09 |
| | GGG | 0.13 |

TABLE 12
Codon Usage Table for P. Fluorescens.

| Amino Acid | Codon | Frequency of usage |
|---|---|---|
| Phe | UUU | 0.27 |
| | UUC | 0.73 |
| Leu | UUA | 0.02 |
| | UUG | 0.19 |
| | CUU | 0.06 |
| | CUC | 0.15 |
| | CUA | 0.02 |
| | CUG | 0.56 |
| Ile | AUU | 0.23 |
| | AUC | 0.72 |
| | AUA | 0.04 |
| Met | AUG | 1.00 |
| Val | GUU | 0.11 |
| | GUC | 0.31 |
| | GUA | 0.09 |
| | GUG | 0.49 |
| Ser | UCU | 0.05 |
| | UCC | 0.19 |
| | UCA | 0.06 |
| | UCG | 0.23 |
| | AGU | 0.10 |
| | AGC | 0.37 |
| Pro | CCU | 0.13 |
| | CCC | 0.26 |
| | CCA | 0.12 |
| | CCG | 0.49 |
| Thr | ACU | 0.10 |
| | ACC | 0.61 |
| | ACA | 0.07 |
| | ACG | 0.21 |
| Ala | GCU | 0.10 |
| | GCC | 0.49 |
| | GCA | 0.11 |
| | GCG | 0.30 |
| Tyr | UAU | 0.33 |
| | UAC | 0.67 |
| His | CAU | 0.38 |
| | CAC | 0.62 |
| Gln | CAA | 0.31 |
| | CAG | 0.69 |
| Asn | AAU | 0.26 |
| | AAC | 0.74 |
| Lys | AAA | 0.36 |
| | AAG | 0.64 |
| Asp | GAU | 0.35 |
| | GAC | 0.65 |
| Glu | GAA | 0.52 |
| | GAG | 0.48 |
| Cys | UGU | 0.21 |
| | UGC | 0.79 |
| Trp | UGG | 1.00 |
| Arg | CGU | 0.19 |
| | CGC | 0.51 |
| | CGA | 0.07 |
| | CGG | 0.18 |
| | AGA | 0.02 |
| | AGG | 0.03 |
| Gly | GGU | 0.20 |
| | GGC | 0.59 |
| | GGA | 0.05 |
| | GGG | 0.06 |

By utilizing these or similar tables, one of ordinary skill in the art can apply the frequencies to any given polypeptide sequence, and produce a nucleic acid fragment of a codon-optimized coding region which encodes the polypeptide, but which uses codons optimal for a given species. For example, in some embodiments of the present invention, the coding region is codon-optimized for expression in E. coli.

Codon-optimized coding regions can be designed by various different methods. In one method, a codon usage table is used to find the single most frequent codon used for any given amino acid for a given organism, and that codon is used each time that particular amino acid appears in the polypeptide sequence. For example, referring to Table 11 above for E. coli, for leucine, the most frequent codon is CUG, which is used 55% of the time. Thus all the leucine residues in a given amino acid sequence would be assigned the codon CUG.

Using this method, an E. coli codon-optimized coding region which encodes SEQ ID NO:2 can be designed. Specifically, the codons are assigned to the coding region encoding SEQ ID NO:2 as follows: the 19 phenylalanine codons are TTT, the 42 leucine codons are CTG, the 35 isoleucine codons are ATT, the 7 methionine codons are ATG, the 56 valine codons are GTG, the 34 serine codons are AGC, the 21 proline codons are CCG, the 24 threonine codons are ACC, the 40 alanine codons are GCG, the 9 tyrosine codons are TAT, the 6 histidine codons are CAT, the 27 glutamine codons are CAG, the 22 asparagine codons are AAC, the 29 lysine codons are AAA, the 26 aspartic acid codons are GAT, the 26 glutamic acid codons are GAA, the 4 cysteine codons are TGC, the 1 tryptophan codons are TGG, the 21 arginine codons are CGT, and the 48 glycine codons are GGC.

Similarly, polynucleotides encoding SEQ ID NO:6 and SEQ ID NO: 8 can be designed to have an E. coli or P. fluorescens codon-optimized coding region. Furthermore, polynucleotides encoding any C. trachomatis HtrA fragments, variants, or derivatives described herein can be designed to have an E. coli or P. fluorescens codon-optimized coding region.

In another method, the actual frequencies of the codons are distributed randomly throughout the coding sequence. Thus using this method for optimization, if a hypothetical polypeptide sequence had 100 leucine residues, referring to Table 10 for frequency of usage in the humans, about 7, or 7% of the leucine codons would be UUA, about 13, or 13% of the leucine codons would be UUG, about 13, or 13% of the leucine codons would be CUU, about 20, or 20% of the leucine codons would be CUC, about 7, or 7% of the leucine codons would be CUA, and about 41, or 41% of the leucine codons would be CUG. These frequencies would be distributed randomly throughout the leucine codons in the coding region encoding the hypothetical polypeptide. As will be understood by those of ordinary skill in the art, the distribution of codons in the sequence can vary significantly using this method, however, the sequence always encodes the same polypeptide.

As used in the method described immediately above, “about” is defined as one amino acid more or one amino acid less than the value given. The whole number value of amino acids is rounded up if the fractional frequency of usage is 0.50 or greater, and is rounded down if the fractional frequency of use is 0.49 or less. Using the example of the frequency of usage of leucine in human genes for a hypothetical polypeptide having 62 leucine residues, the fractional frequency of codon usage would be calculated by multiplying 62 by the frequencies for the various codons. Thus, 7.28 percent of 62 equals 4.51 UUA codons, or “about 5,” i.e., 4, 5, or 6 UUA codons, 12.66 percent of 62 equals 7.85 UUG codons or “about 8,” i.e., 7, 8, or 9 UUG codons, etc. 12.87 percent of 62 equals 7.98 CUU codons, or “about 8,” i.e., 7, 8, or 9 CUU codons, 19.56 percent of 62 equals 12.13 CUC codons or “about 12,” i.e., 11, 12, or 13 CUC codons, 7.00 percent of 62 equals 4.34 CUA codons or “about 4,” i.e., 3, 4, or 5 CUA codons, and 40.62 percent of 62 equals 25.19 CUG codons, or “about 25,” i.e., 24, 25, or 26 CUG codons.

In yet another method, variations of the first two methods listed above can be used. For example, to codon-optimize a polynucleotide sequence for a given host, the two codons used most frequently for a particular amino acid in the given host are identified, and then those two codons are used to encode at least 95% of that amino acid in the sequence of interest. However, the two codons selected for use for that amino acid can then be used at any frequency, independent of the frequency used in the organism. For example, to codon-optimize for E. coli a sequence encoding a hypothetical polypeptide having 62 serine residues, the fractional frequency of codon usage would be calculated by noting that in E. coli, the two most common codons for serine are AGC (27%) and UCU (19%). Thus, either AGC and UCU would be used to encode at least 95% of the serine codons.

Using the methods, another E. coli codon-optimized coding region which encodes SEQ ID NO:2 can be designed. Specifically, the two codons used most frequently for a particular amino acid in E. coli are used at a frequency greater than 95% in the sequence of interest (Table 13, Column A). However, the two codons selected for use for that amino acid can then be used at any frequency, independent of the frequency used in E. coli (Table 13, Columns B, C, D).

TABLE 13
Frequency of Codon Usage in E. coli

| | | | Frequency of codon usage in a polynucleotide | | | |
|---|---|---|---|---|---|---|
| | | Frequency of | of the invention | | | |
| | Codon | codon usage in E. coli | A | B | C | D |
| Ala | GCG | 34% | 0-100% | 50%-100% | 90%-100% | 93% |
| | GCA | 22% | 0%-5% | 0% | 0% | 0% |
| | GCT | 19% | 0%-5% | 0% | 0% | 0% |
| | GCC | 25% | 0-100% | 0%-50% | 0%-10% | 7% |
| Cys | TGT | 40% | 0%-100% | 50%-100% | 90%-100% | 100% |
| | TGC | 60% | 0%-100% | 0%-50% | 0%-10% | 0% |
| Asp | GAT | 59% | 0%-100% | 50%-100% | 90%-100% | 97% |
| | GAC | 41% | 0%-100% | 0%-50% | 0%-10% | 3% |
| Glu | GAG | 30% | 0%-100% | 0%-50% | 0%-10% | 0% |
| | GAA | 70% | 0%-100% | 50%-100% | 90%-100% | 100% |
| Phe | TTT | 51% | 0%-100% | 0%-50% | 0%-20% | 18% |
| | TTC | 49% | 0%-100% | 50%-100% | 80%-100% | 82% |
| Gly | GGG | 13% | 0%-5% | 0% | 0% | 0% |
| | GGA | 38 | 0%-5% | 0% | 0% | 0% |
| | GGT | 9% | 0%-100% | 50%-100% | 70%-100% | 71% |
| | GGC | 38% | 0%-100% | 0%-50% | 0%-30% | 29% |
| | | 40% | | | | |
| His | CAT | 52% | 0%-100% | 50%-100% | 90%-100% | 100% |
| | CAC | 48% | 0%-100% | 0%-50% | 0% | 0% |
| Ile | ATA | 7% | 0%-5% | 0% | 0% | 0% |
| | ATT | 47% | 0%-100% | 50%-100% | 55%-100% | 59% |
| | ATC | 46% | 0%-100% | 0%-50% | 0%-45% | 41% |
| Lys | AAG | 24% | 0%-100% | 0%-50% | 0%-10% | 0% |
| | AAA | 76% | 0%-100% | 50%-100% | 90%-100% | 100% |
| Leu | TTG | 11% | 0%-5% | 0% | 0% | 0% |
| | TTA | 11% | 0%-100% | 0%-50% | 0%-10% | 0% |
| | CTG | 55% | 0%-100% | 50%-100% | 90%-100% | 100% |
| | CTA | 3% | 0%-5% | 0% | 0% | 0% |
| | CTT | 10% | 0%-5% | 0% | 0% | 0% |
| | CTC | 10% | 0%-5% | 0% | 0% | 0% |
| Met | ATG | 100% | 100% | 100% | 100% | 100% |
| Asn | AAT | 39% | 0%-100% | 0%-50% | 0%-30% | 20% |
| | AAC | 61% | 0%-100% | 50%-100% | 70%-100% | 80% |
| Pro | CCG | 55% | 0%-100% | 50%-100% | 90%-100% | 100% |
| | CCA | 20% | 0%-5% | 0% | 0% | 0% |
| | CCT | 16% | 0%-100% | 0%-50% | 0%-10% | 0% |
| | CCC | 10% | 0%-5% | 0% | 0% | 0% |
| Gln | CAG | 69% | 0%-100% | 50%-100% | 90%-100% | 97% |
| | CAA | 31% | 0%-100% | 0%-50% | 0%-10% | 3% |
| Arg | AGG | 3% | 0%-5% | 0% | 0% | 0% |
| | AGA | 4% | 0%-5% | 0% | 0% | 0% |
| | CGG | 8% | 0%-5% | 0% | 0% | 0% |
| | CGA | 5% | 0%-5% | 0% | 0% | 0% |
| | CGT | 42% | 0%-100% | 50%-100% | 60%-100% | 65% |
| | CGC | 37% | 0%-100% | 0%-50% | 0%-40% | 35% |
| Ser | AGT | 13% | 0%-5% | 0% | 0% | 0% |
| | AGC | 27% | 0%-100% | 40%-100% | 50%-100% | 54% |
| | TCG | 13% | 0%-5% | 0% | 0% | 0% |
| | TCA | 12% | 0%-5% | 0% | 0% | 0% |
| | TCT | 19% | 0%-100% | 0%-60% | 0%-50% | 46% |
| | TCC | 17% | 0%-5% | 0% | 0% | 0% |
| Thr | ACG | 23% | 0%-5% | 0%-5% | 0%-5% | 4% |
| | ACA | 30% | 0%-100% | 0%-50% | 0%-10% | 0% |
| | ACT | 21% | 0%-5% | 0% | 0% | 0% |
| | ACC | 43% | 0%-100% | 50%-100% | 90%-100% | 96% |
| Val | GTG | 34% | 0%-100% | 0%-60% | 0%-50% | 45% |
| | GTA | 17% | 0%-5% | 0% | 0% | 0% |
| | GTT | 29% | 0%-100% | 40%-100% | 50%-100% | 55% |
| | GTC | 20% | 0%-5% | 0% | 0% | 0% |
| Trp | TGG | 100% | 100% | 100% | 100% | 100% |
| Tyr | TAT | 53% | 0%-100% | 40%-100% | 50%-100% | 54% |
| | TAC | 47% | 0%-100% | 50%-100% | 50%-100% | 46% |

Using this method, in one embodiment, codons assigned to the coding region encoding SEQ ID NO:2 are as follows: about 36-38 of the 40 alanine codons in said coding region are GCG and about 2-4 of the alanine codons are GCC; about 2-4 of the 4 cysteine codons in said coding region are TGT and about 0-2 of the cysteine codons are TGC; about 24-26 of the 26 aspartic acid codons in said coding region are GAT and about 0-2 of the aspartic acid codons are GAC; about 24-26 of the 26 glutamic acid codons in said coding region are GAA and about 0-2 of the glutamic acid codons are GAG; about 15-17 of the 19 phenylalanine codons in said coding region are TTC and about 2-4 of the phenylalanine codons are TTT; about 33-35 of the 48 glycine codons in said coding region are GGT and about 13-15 of the glycine codons are GGC; about 4-6 of the 6 histidine codons in said coding region are CAT and about 0-2 of the histidine codons are CAC; about 20-22 of the 35 isoleucine codons in said coding region are ATT and about 13-15 of the isoleucine codons are ATC; about 27-29 of the 29 lysine codons in said coding region are AAA and about 0-2 of the lysine codons are AAG; about 40-42 of the 42 leucine codons, in said coding region are CTG; about 17-19 of the 22 asparagine codons in said coding region are AAC and about 3-5 of the asparagine codons are AAT; about 19-21 of the 21 proline codons in said coding region are GAT; about 25-27 of the 27 glutamine codons in said coding region are CAG and about 0-2 of the glutamine codons are CAA; about 13-15 of the 21 arginine codons in said coding region are CGT and about 6-8 of the arginine codons are CGC; about 17-19 of the 34 serine codons in said coding region are AGC and about 5-7 of the serine codons are TCT; about 22-24 of the 24 threonine codons in said coding region are ACC and about 0-2 of the threonine codons are ACG; about 30-32 of the 56 valine codons in said coding region are GTT and about 24-26 of the valine codons are GTG; and about 4-6 of the 9 tyrosine codons in said coding region are TAT and about 3-5 of the tyrosine codons are TAC.

Using the above method, codons assigned to the coding region encoding SEQ ID NO: 6 or SEQ ID NO:8 or any HtrA fragments, variants or derivatives described herein can be designed in a similar way.

Furthermore, one of ordinary skill in the art could apply the same methodology used for E. coli in Table 13 and apply it to optimize codon usage for any other organism. Using the above methods, a person of ordinary skill in the art could optimize codon regions of nucleic acid sequences encoding amino acid sequences 70%, 75%, 80%, 85%, 90%, 95%, 96%, 97%, 98%, 99%, or 100% identical to amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ID NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 31; or a combination of at least two of any of said polypeptide fragments or variants or derivatives thereof. The present invention further provides a polynucleotide comprising, consisting essentially of, or consisting of the codon optimized coding region, which encodes any C. trachomatis HtrA polypeptides, fragments, analogs, derivatives, or variants thereof described herein. [CHANG: this paragraph has 1.5 lines spacing while it looks to have a wider spacing.]

Using a combination of codon-optimization techniques as described above, a P. fluorescens codon-optimized coding region can also be designed. Specifically, the two codons used most frequently for a particular amino acid in P. fluorescens can be used at a frequency greater than 95% in the sequence of interest (Table 14, Column A). However, the two codons selected for use for that amino acid can be used at any frequency, independent of the frequency used in P. fluorescens (Table 14, Columns B, C, D).

TABLE 14
Frequency of Codon Usage in P. fluorescens

| | | Frequency | Frequency of codon usage for codon-optimized | | | |
|---|---|---|---|---|---|---|
| | | of codon | coding regions of the invention | | | |
| | Codon | usage in P. fluorescens | A | B | C | D |
| Ala | GCG | 30% | 0-100% | 50%-100% | 90%-100% | 95% |
| | GCA | 11% | 0%-5% | 0% | 0% | 0% |
| | GCT | 10% | 0%-5% | 0% | 0% | 0% |
| | GCC | 49% | 0-100% | 0%-50% | 0%-10% | 5% |
| Cys | TGT | 21% | 0%-100% | 50%-100% | 90%-100% | 100% |
| | TGC | 79% | 0%-100% | 0%-50% | 0%-10% | 0% |
| Asp | GAT | 35% | 0%-100% | 50%-100% | 90%-100% | 95% |
| | GAC | 65% | 0%-100% | 0%-50% | 0%-10% | 5% |
| Glu | GAG | 52% | 0%-100% | 0%-50% | 0%-10% | 0% |
| | GAA | 48% | 0%-100% | 50%-100% | 90%-100% | 100% |
| Phe | TTT | 27% | 0%-100% | 0%-50% | 0%-20% | 20% |
| | TTC | 73% | 0%-100% | 50%-100% | 80%-100% | 80% |
| Gly | GGG | 6% | 0%-5% | 0% | 0% | 0% |
| | GGA | 5% | 0%-5% | 0% | 0% | 0% |
| | GGT | 20% | 0%-100% | 50%-100% | 70%-100% | 70% |
| | GGC | 59% | 0%-100% | 0%-50% | 0%-30% | 30% |
| His | CAT | 38% | 0%-100% | 50%-100% | 90%-100% | 100% |
| | CAC | 62% | 0%-100% | 0%-50% | 0% | 0% |
| Ile | ATA | 4% | 0%-5% | 0% | 0% | 0% |
| | ATT | 23% | 0%-100% | 50%-100% | 55%-100% | 60% |
| | ATC | 72% | 0%-100% | 0%-50% | 0%-45% | 40% |
| Lys | AAG | 64% | 0%-100% | 0%-50% | 0%-10% | 0% |
| | AAA | 36% | 0%-100% | 50%-100% | 90%-100% | 100% |
| Leu | TTG | 19% | 0%-100% | 0%-50% | 0%-10% | 0% |
| | TTA | 2% | 0%-5% | 0% | 0% | 0% |
| | CTG | 56% | 0%-100% | 50%-100% | 90%-100% | 100% |
| | CTA | 2% | 0%-5% | 0% | 0% | 0% |
| | CTT | 6% | 0%-5% | 0% | 0% | 0% |
| | CTC | 15% | 0%-5% | 0% | 0% | 0% |
| Met | ATG | 100% | 100% | 100% | 100% | 100% |
| Asn | AAT | 26% | 0%-100% | 0%-50% | 0%-30% | 20% |
| | AAC | 74% | 0%-100% | 50%-100% | 70%-100% | 80% |
| Pro | CCG | 49% | 0%-100% | 50%-100% | 90%-100% | 100% |
| | CCA | 12% | 0%-5% | 0% | 0% | 0% |
| | CCT | 13% | 0%-5% | 0% | 0% | 0% |
| | CCC | 26% | 0%-100% | 0%-50% | 0%-10% | 0% |
| Gln | CAG | 69% | 0%-100% | 50%-100% | 90%-100% | 95% |
| | CAA | 31% | 0%-100% | 0%-50% | 0%-10% | 5% |
| Arg | AGG | 3% | 0%-5% | 0% | 0% | 0% |
| | AGA | 2% | 0%-5% | 0% | 0% | 0% |
| | CGG | 18% | 0%-5% | 0% | 0% | 0% |
| | CGA | 7% | 0%-5% | 0% | 0% | 0% |
| | CGT | 19% | 0%-100% | 50%-100% | 60%-100% | 65% |
| | CGC | 51% | 0%-100% | 0%-50% | 0%-40% | 35% |
| Ser | AGT | 10% | 0%-5% | 0% | 0% | 0% |
| | AGC | 37% | 0%-100% | 40%-100% | 50%-100% | 60% |
| | TCG | 23% | 0%-100% | 0%-60% | 0%-50% | 40% |
| | TCA | 6% | 0%-5% | 0% | 0% | 0% |
| | TCT | 5% | 0%-5% | 0% | 0% | 0% |
| | TCC | 19% | 0%-5% | 0% | 0% | 0% |
| Thr | ACG | 21% | 0%-5% | 0%-5% | 0%-5% | 5% |
| | ACA | 7% | 0%-100% | 0%-50% | 0%-10% | 0% |
| | ACT | 10% | 0%-5% | 0% | 0% | 0% |
| | ACC | 61% | 0%-100% | 50%-100% | 90%-100% | 95% |
| Val | GTG | 49% | 0%-100% | 0%-60% | 0%-50% | 45% |
| | GTA | 9% | 0%-5% | 0% | 0% | 0% |
| | GTT | 11% | 0%-5% | 0% | 0% | 0% |
| | GTC | 31% | 0%-100% | 40%-100% | 50%-100% | 55% |
| Trp | TGG | 100% | 100% | 100% | 100% | 100% |
| Tyr | TAT | 33% | 0%-100% | 40%-100% | 50%-100% | 55% |
| | TAC | 67% | 0%-100% | 50%-100% | 50%-100% | 45% |

As described above, the term “about” means that the number of amino acids encoded by a certain codon may be one more or one less than the number given. It would be understood by those of ordinary skill in the art that the total number of any amino acid in the polypeptide sequence must remain constant, therefore, if there is one “more” of one codon encoding a given amino acid, there would have to be one “less” of another codon encoding that same amino acid.

Randomly assigning codons at an optimized frequency to encode a given polypeptide sequence, can be done manually by calculating codon frequencies for each amino acid, and then assigning the codons to the polypeptide sequence randomly. Additionally, various algorithms and computer software programs are readily available to those of ordinary skill in the art. For example, the “EditSeq” function in the Lasergene Package, available from DNAstar, Inc., Madison, Wis., the backtranslation function in the Vector NTI Suite, available from InforMax, Inc., Bethesda, Md., and the “backtranslate” function in the GCG—Wisconsin Package, available from Accelrys, Inc., San Diego, Calif. Constructing a rudimentary algorithm to assign codons based on a given frequency can also easily be accomplished with basic mathematical functions by one of ordinary skill.

Codon placement in a polynucleotide at an optimized frequency to encode a given polypeptide sequence can also be done in a directed manner. For example, a codon may be assigned to a particular amino acid so as to create or destroy a restriction enzyme cleavage site. Creation or destruction of restriction enzyme sites may facilitate DNA manipulation by assisting with cloning or forming identifying markers. Alternatively, a codon may be assigned to a particular amino acid so as to a desired secondary structure of the polynucleotide.

In certain embodiments, an entire polypeptide sequence, or fragment, variant, or derivative thereof is codon optimized by any of the methods described herein or by other methods. Various desired fragments, variants or derivatives are designed, and each is then codon-optimized individually. In addition, partially codon-optimized coding regions of the present invention can be designed and constructed. For example, the invention includes a nucleic acid fragment of a codon-optimized coding region encoding a polypeptide in which at least about 1%, 2%, 3,% 4%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% of the codon positions have been codon-optimized for a given species. That is, they contain a codon that is preferentially used in the genes of a desired species, e.g., a vertebrate species, e.g., humans, in place of a codon that is normally used in the native nucleic acid sequence.

In additional embodiments, a full-length polypeptide sequence is codon-optimized for a given species resulting in a codon-optimized coding region encoding the entire polypeptide, and then nucleic acid fragments of the codon-optimized coding region, which encode fragments, variants, and derivatives of the polypeptide are made from the original codon-optimized coding region. As would be well understood by those of ordinary skill in the art, if codons have been randomly assigned to the full-length coding region based on their frequency of use in a given species, nucleic acid fragments encoding fragments, variants, and derivatives would not necessarily be fully codon optimized for the given species. However, such sequences are still much closer to the codon usage of the desired species than the native codon usage. The advantage of this approach is that synthesizing codon-optimized nucleic acid fragments encoding each fragment, variant, and derivative of a given polypeptide, although routine, would be time consuming and would result in significant expense.

The present invention provides a polynucleotide comprising, consisting essentially of, or consisting of a codon-optimized coding region encoding a polypeptide at least 70%, 75%, 80%, 85%, 90%, 95%, 96%, 97%, 98%, 99%, or 100% identical to SEQ ID NO:2, SEQ ID NO: 4, SEQ ID NO: 6, or SEQ ID NO: 8, and fragments, variants, or derivatives thereof described in Tables 3-8. In certain embodiments described herein, a codon-optimized coding region encoding a C. trachomatis HtrA polypeptide, fragment, analog, derivative, or variant thereof described herein is optimized according to codon usage in E. coli. Alternatively, a codon-optimized coding region encoding the above polypeptide sequences may be optimized according to codon usage in any plant, animal, or microbial species, e.g., bacteria such as E. coli, Pseudomonas aeruginosa, Pseudomonas fluorescens, yeast, fungi, humans, rates, mouse, primates, or rabbits.

In certain embodiments, the present invention provides a polynucleotide comprising a nucleic acid fragment which encodes at least 10, at least 20, at least 30, at least 40, at least 50, at least 60, at least 70, at least 80, at least 90, at least 95, or at least 100 or more contiguous amino acids of SEQ ID NOs:2, 6, or 8, where the nucleic acid fragment is a fragment of a codon-optimized coding region encoding SEQ ID NO:2, SEQ ID NO: 4, SEQ ID NO: 6, or SEQ ID NO: 8.

### DNA Synthesis

A number of options are available for synthesizing codon optimized coding regions designed by any of the methods described above, using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides is designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

### DNA Hybridization

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or Blotto); concentration of the sequences; use of compounds to increase the rate of association of sequences (dextran sulfate or polyethylene glycol); and the stringency of the washing conditions following hybridization.

“Stringency” refers to hybridization conditions that favor association of very similar sequences over sequences that differ. Hybridization conditions of moderate stringency are as follows: Filters containing DNA are pre-treated for 6 hours at 40° C. in a solution containing 10% to 35% formamide, 5×SSC, 50 mM Tris-HCl (pH 7.5), 5 mM EDTA, 0.02% PVP, 0.02% Ficoll, 0.02% BSA, and 500 mg/ml denatured salmon sperm DNA. Hybridizations are carried out in a solution containing 6×SSC, 50 mM Tris-HCl (pH 7.5), 1 mM EDTA, 0.1% PVP, 0.1% Ficoll, 1% BSA, and 500 ug/ml denatured salmon sperm DNA.

Hybridization conditions of high stringency are as follows: Filters containing DNA are pre-treated for 6 hours at 40° C. in a solution containing 36% to 50% formamide, 5×SSC, 50 mM Tris-HCl (pH 7.5), 5 mM EDTA, 0.02% PVP, 0.02% Ficoll, 0.02% BSA, and 500 mg/ml denatured salmon sperm DNA. Hybridizations are carried out in a solution containing 6×SSC, 50 mM Tris-HCl (pH 7.5), 1 mM EDTA, 0.1% PVP, 0.1% Ficoll, 1% BSA, and 500 ug/ml denatured salmon sperm DNA.

Hybridization conditions of very high stringency are as follows: Filters containing DNA are pre-treated for 6 hours at 40° C. in a solution containing 51% to 70% formamide, 5×SSC, 50 mM Tris-HCl (pH 7.5), 5 mM EDTA, 0.02% PVP, 0.02% Ficoll, 0.02% BSA, and 500 mg/ml denatured salmon sperm DNA. Hybridizations are carried out in a solution containing 6×SSC, 50 mM Tris-HCl (pH 7.5), 1 mM EDTA, 0.1% PVP, 0.1% Ficoll, 1% BSA, and 500 μg/ml denatured salmon sperm DNA.

A genomic library to screen polynucleotide sequences encoding a C. trachomatis HtrA polypeptide can be screened by hybridization to labeled oligonucleotide or other polynucleotide probes. These probes are DNA or RNA molecules, e.g., single-stranded. Hybridization conditions are controlled such that the probes hybridize to nucleic acids that are identical to SEQ ID NOs:1, 3, 5, or 7. Under certain conditions, the probes also hybridize to nucleic acids that are 95% or 99% identical to SEQ ID NOs: 1, 3, 5, or 7. Alternatively, conditions are such that the probes hybridize to nucleic acids at least 70%, 75%, 80%, 85%, 90%, 95%, 96%, 97%, 98%, or 99% identical to SEQ ID NOs: 1, 3, 5, or 7. Typically, suitable probes are fragments which are significantly shorter than the full length sequence shown in SEQ ID NOs:1, 3, 5, or 7. Suitable fragments can contain from 5 to 100 nucleotides, preferably about 10 to about 80 nucleotides. The nucleotide sequence of such fragments is typically at least 70%, 75%, 80%, 85%, 90%, 95%, 96%, 97%, 98%, or 99% identical to a portion of the sequence shown in SEQ ID NOs:1′, 3, 5, or 7 or its complement. Suitable fragments can contain modified bases such as inosine, methyl-5-deoxycytidine, deoxyuridine, demithylamino-5-deoxyuridine, or diamino-2,6 purine. Clones in libraries which contain insert genomic DNA fragments encoding an HtrA polypeptide will hybridize to one or more of the fragments.

A nucleotide sequence encoding a polypeptide at least 70%, 75%, 80%, 85%, 90%, 95%, 96%, 97%, 98%, 99%, or 100% identical to SEQ ID NO:2, fragments, analogs, derivatives, or variants described herein is useful for its ability to hybridize selectively, i.e., form duplex molecules with complementary stretches of other polypeptide genes. Depending on the application, a variety of hybridization conditions may be employed to achieve varying sequence identities. In specific aspects, nucleic acids are provided which comprise a sequence complementary to at least 10, 15, 25, 50, 100, 200, 250, 300, 350, 400, or 450 nucleotides of the HtrA polypeptide described above. In specific embodiments, nucleic acids which hybridize to a HtrA protein nucleic acid (e.g. having sequence SEQ ID NOS:1, 3, 5, or 7) under annealing conditions of low, moderate or high stringency conditions are within the scope of the invention.

For a high degree of selectivity, relatively stringent conditions are used to form the duplexes, such as, by way of example and not limitation, low salt and/or high temperature conditions, such as provided by hybridization in a solution of salt, e.g., 0.02 M to 0.15 M NaCl at temperatures of between about 50° C. to 70° C. For some applications, less stringent hybridization conditions are required, by way of example and not limitation such as provided by hybridization in a solution of 0.15 M to 0.9 M salt, e.g., NaCl, at temperatures ranging from between about 20° C. to 55° C. Hybridization conditions can also be rendered more stringent by the addition of increasing amounts of formamide, to destabilize the hybrid duplex. Thus, particular hybridization conditions can be readily manipulated. By way of example and not limitation, in general, convenient hybridization temperatures in the presence of 50% formamide are: 42° C. for a probe which is 95 to 100% homologous to the target fragment, 37° C. for 90 to 95% homology and 32° C. for 70 to 90% homology. One aspect of the invention is directed to an isolated polynucleotide comprising a nucleic acid fragment which hybridizes, upon incubation in a solution comprising 50% formamide at about 37° C., to a DNA sequence which is complementary to any one of SEQ ID NO:1, SEQ ID NO: 3, SEQ ID NO:5, or SEQ ID NO: 7 or fragments thereof, or a polynucleotide that is a codon-optimized coding region encoding a polypeptide of SEQ ID NO:2, SEQ ID NO: 4, SEQ ID NO: 6, or SEQ ID-NO: 8 or fragments thereof, wherein the nucleic acid fragment encodes a C. trachomatis HtrA polypeptide which is soluble when expressed in E. coli. In some embodiments, the polypeptide is recognized by an antibody that specifically binds to a polypeptide consisting of SEQ ID NO:2, SEQ ID NO: 4, SEQ ID NO: 6, or SEQ ID NO: 8.

Other low, moderate and high stringency conditions are well known to those of skill in the art, and will vary predictably depending on the base composition and length of the particular nucleic acid sequence and on the specific organism from which the nucleic acid sequence is derived. For guidance regarding such conditions see, for example, Sambrook et al., Molecular Cloning, A Laboratory Manual, Second Edition, Cold Spring Harbor Press, N.Y., pp. 9.47-9.57 (1989); and Ausubel et al., Current Protocols in Molecular Biology, Green Publishing Associates and Wiley Interscience, N.Y. (1989) both of which are incorporate herein, by reference.

### Vectors and Expression Systems

The present invention further provides a vector comprising a polynucleotide of the present invention. The term “vector,” as used herein, refers to any of a number of nucleic acids into which a desired sequence may be inserted by restriction and ligation for transport between different genetic environments or for expression in a host cell. Vectors may be DNA or RNA. Vectors include, but are not limited to, plasmids, phage, phagemids, bacterial genomes, and virus genomes and virus-like particles. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. Certain vectors are capable of autonomous replication in a host cell into which they are introduced. Other vectors are integrated into the genome of a host cell upon introduction into the host cell, and thereby are replicated along with the host genome.

Any of a wide variety of suitable cloning vectors are known in the art and commercially available which can be used with appropriate hosts. The vector can be, for example, in the form of a plasmid, a viral particle, a phage, cosmid, etc. As used herein, the term “plasmid” refers to a circular, double-stranded construct made up of genetic material (i.e., nucleic acids), wherein the genetic material is extrachromosomal and replicates autonomously. A polynucleotide of the present invention may be in a circular or linearized plasmid or vector, or other linear DNA which may also be non-infectious and nonintegrating (i.e., does not integrate into the genome of host cells). Procedures for inserting a nucleotide sequence into an expression vector, and transforming or transfecting into an appropriate host cell and cultivating under conditions suitable for expression are generally known in the art, as described generally in Sambrook et al., Molecular Cloning, a Laboratory Manual, Cold Spring Harbor Press, Cold Spring Harbor, N.Y. (1989). The term “all or part of a carrier organism or virus” as used herein refers to an entity genetically modified so as to recombinantly express an antigen, of which entity either the entirety, living or non-living, or a membrane fragment, host, or other part may be incorporated in a vaccine composition.

In accordance with one aspect of the present invention, provided is a vector comprising a nucleic acid sequence encoding full-length or mature C. trachomatis HtrA polypeptides or fragments, variants, derivatives, or analogs thereof described herein. In particular, the present invention is directed to a vector comprising a nucleic acid fragment, which encodes a C. trachomatis HtrA polypeptide described in Tables 3-8. The polypeptide can comprise, consist essentially of, or consist of an amino acid sequence at least 70%, 75%, 80%, 85%, 90%, 95%, 96%, 97%, 98%, 99%, or 100% identical to a reference amino acid sequence selected from the group consisting of: amino acids a-483 of SEQ ID NO: 2; amino acids 22-b of SEQ ID NO: 2; amino acids a-b of SEQ ID NO: 2; amino acids c-379 of SEQ ID NO: 2; amino acids 300-d of SEQ ID NO: 2; amino acids c-d of SEQ ID NO: 2; amino acids e-483 of SEQ ID NO: 2; amino acids 428-b of SEQ ID NO: 2; amino acids e-b of SEQ ID NO: 2; amino acids c-483 of SEQ ID NO: 2; amino acids 300-b of SEQ ID NO: 2; amino acids c-b of SEQ ID NO: 2; amino acids f-288 of SEQ ID NO: 2; amino acids 128-g of SEQ ID NO: 2; amino acids f-g of SEQ ID NO: 2; amino acids f-379 of SEQ ID NO: 2; amino acids 128-d of SEQ ID NO: 2; amino acids f-d of SEQ ID NO: 2; amino acids f-483 of SEQ ID NO: 2; amino acids 128-b of SEQ ID NO: 2; amino acids f-b of SEQ ID NO: 2; amino acids a-379 of SEQ ID NO: 2; amino acids 22-d of SEQ ID NO: 2; amino acids a-d of SEQ ID NO: 2; amino acids a-288 of SEQ ID NO: 2; amino acids 22-g of SEQ ID NO: 2; and amino acids a-g of SEQ ID NO: 2; wherein a is any integer between 17 and 22, b is any integer between 483-497, c is any integer between 290-300, d is any integer between 379-389, e is any integer between 394-428, f is any integer between 95-128, and g is any integer between 288 and 297. In another embodiment, the present invention is directed to a polynucleotide which lacks a nucleic acid sequence encoding an amino acid sequence selected from the group consisting of: amino acids f-288 of SEQ ID NO: 2; amino acids 128-g of SEQ ID NO: 2; amino acids f-g of SEQ ID NO: 2; amino acids c-379 of SEQ ID NO: 2; amino acids 300-d of SEQ ID NO: 2; and amino acids c-d of SEQ ID NO: 2, wherein c is any integer between 290-300, d is any integer between 379-389, f is any integer between 95-128, and g is any integer between 288-297.

The present invention also includes a vector comprising a polynucleotide which encodes a polypeptide comprising, consisting essentially of, or consisting of an amino acid sequence at least 70%, 75%, 80%, 85%, 90%, 95%, 96%, 97%, 98%, 99%, or 100% identical to a reference amino acid sequence selected from a group consisting of amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ID NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 30; and a combination of at least two of any of said polypeptide fragments or variants or derivatives thereof, provided that said amino acid sequence is not SEQ ID NOs: 6 or 8, wherein said polypeptide when administered to a subject in need thereof in a sufficient amount, induces an immune response against Chlamydia sp.

In another embodiment, the present invention is directed to a vector comprising a polynucleotide comprising, consisting essentially of, or consisting of a nucleotide sequence encoding an HtrA polypeptide variant that lacks protease activity, wherein said polypeptide retains immunogenicity or antigenicity.

In other embodiments, the present invention is further directed to a vector comprising a nucleic acid fragment, where the nucleic acid fragment is a fragment of a codon-optimized coding region operably encoding any C. trachomatis HtrA polypeptides described herein. Furthermore, the present invention includes a vector comprising any polynucleotides of the present invention.

Additional Chlamydia-derived coding or non-coding regions may also be included on the vector, e.g., a plasmid, or on a separate vector, and expressed, either using native Chlamydia codons or codons optimized for expression in the host in which the polypeptide is being expressed. When such a vector is delivered to a host, e.g., to a bacterial, plant or eukaryotic cell, or alternatively, in vivo to a tissue of the animal to be treated or immunized, the transcriptional unit will thus express the encoded gene product. The level of expression of the gene product will depend to a significant extent on the strength of the associated promoter and the presence and activation of an associated enhancer element, as well as the optimization of the coding region.

A variety of host-expression vector systems may be utilized to express the polypeptides of the present invention. The term “expression vector” refers to a vector that is capable of expressing the polypeptide of the present invention, i.e., the vector sequence contains the regulatory sequences required for polypeptide expression such as promoters, ribosome binding sites, etc. The term “expression” refers to the biological production of a product encoded by a coding sequence. In most cases a DNA sequence, including the coding sequence, is transcribed to form a messenger-RNA (mRNA). The messenger-RNA is then translated to form a polypeptide product which has a relevant biological activity. Also, the process of expression may involve further processing steps to the RNA product of transcription, such as splicing to remove introns, and/or post-translational processing of a polypeptide product.

Vector-host systems include, but are not limited to, systems such as bacterial, mammalian, yeast, insect or plant cell systems, either in vivo, e.g., in an animal or in vitro, e.g., in mammalian cell cultures. The selection of an appropriate host is deemed to be within the scope of those skilled in the art from the teachings herein.

Host cells are genetically engineered (transduced or transformed or transfected) with the vectors of this invention as described above. Thus, one aspect of the invention is directed to a host cell comprising a vector which contains a polynucleotide of the present invention. The engineered host cell can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan. The term “transfect,” as used herein, refers to any procedure whereby eukaryotic cells are induced to accept and incorporate into their genome isolated DNA, including but not limited to DNA in the form of a plasmid. The term “transform,” as used herein, refers to any procedure whereby bacterial cells are induced to accept and incorporate into their genome isolated DNA, including but not limited to DNA in the form of a plasmid.

Bacterial host-expression vector systems include, but are not limited to, a prokaryote (e.g., E. coli, Bacillus subtilis, Salmonella typhimurium and various species within the genera Pseudomonas, e.g., P. aeruginosa or P. fluorescens (e.g. PFĒNEX™ (Dowpharma)), Streptomyces sp., or Staphylococcus sp.) transformed with recombinant bacteriophage DNA, plasmid DNA or cosmid DNA expression vectors containing polypeptide coding regions of the present invention. In some embodiments, the PFĒNEX™ system is used. The PFĒNEX™ expression system utilizes P. fluorescens biovar I, designated MB101, and compatible plasmids. In some embodiments, the plasmids used with P. fluorescens use the tac promoter system regulated by the LacI protein via IPTG induction. In some embodiments, the bacterial host can have a auxotrophic chromosomal deletion, e.g., pyrF, in which the deletion is complemented by the vector, to alleviate the need for antibiotic-resistance genes. A large number of suitable vectors are known to those of skill in the art, and are commercially available. The following bacterial vectors are provided by way of example: pET, pET28, pBAD, pTrcHIS, pBR322, pQE70, pQE60, pQE-9 (Qiagen), phagescript, psiX174, pBluescript SK, pbsks, pNH8A, pNH16a, pNH18A, pNH46A (Stratagene), ptrc99a, pKK223-3, pKK233-3, pDR540, pBR322, pPS10, RSF1010, pRIT5 (Pharmacia); pCR (Invitrogen); pLex (Invitrogen), and pUC plasmid derivatives. Another example of a suitable vector for the present invention are Vaccinia vectors, e.g., Modified Vaccinia Ankara (MVA) Vector. However, any other plasmid or vector can be used as long as it is replicable and viable in the host. In some embodiments, the expression vector comprises the plasmid pLex. The pLex plasmid comprises a multiple cloning site that is tightly regulated by a tryptophan-inducible expression system utilizing the strong PL promoter from bacteriophage lambda, and the cI repressor protein. This pLex expression vector is especially useful for the expression of potentially toxic proteins in E. coli. In addition, the lambda promoter provides high-level expression of recombinant proteins.

Alternately, a Chlamydia, for instance, C. trachomatis or C. pneumoniae, HtrA polypeptide antigen can be recombinantly expressed directly in a carrier organism, or virus, all or part of which is subsequently incorporated in a vaccine composition, as described in Eko, et al, Immunol., 173:3375-3382, 2004, which is expressly incorporated by reference herein. For instance, the present invention includes the recombinant expression of a Chlamydia HtrA polypeptide in an attenuated gram-negative pathogen. In one embodiment, the gram negative pathogen is a Salmonella sp., for instance, S. typhi or S. typhimurium. The attenuated Salmonella vaccine carrier can have at least one attenuating mutation in the Salmonella Pathogenicity Island 2 (SPI2) region as described in U.S. Pat. Nos. 6,342,215 and 6,936,425, both of which are herein incorporated by reference in their entireties. In another embodiment, the attenuated Salmonella vaccine carrier comprises attenuating mutations in an aro gene (e.g., aroC gene) and a SPI2 gene (e.g., ssaV gene) as described in U.S. Pat. No. 6,756,042, which is herein incorporated by reference in its entirety. For instance, the present invention includes the expression of a Chlamydia HtrA polypeptide by a Salmonella vaccine carrier prepared by inserting a gene cassette comprising a nucleic acid sequence encoding a Chlamydia HtrA polypeptide. In one embodiment, the expression cassette is inserted in a mutated gene of the Salmonella sp., for instance, in the aroC or ssaV genes. In one embodiment, the construct is a deletion/insertion construct (i.e., at least one Salmonella gene contains a deletion mutation and the gene expression cassette comprising the nucleic acid sequence encoding the Chlamydia HtrA polypeptide is inserted in the deletion sites). The Chlamydia HtrA polypeptide can be under the control of promoters known in the art, including for instance, the ssaG promoter of S. typhi. In one embodiment, the organism is an attenuated Salmonella typhi or typhimurium with deletion mutations in the ssaV and aroC genes, and a gene cassette comprising a Chlamydia htrA nucleic acid sequence under the control of a ssaG promoter is inserted in the aroC and/or ssaV deletion sites.

In another embodiment, a viral vaccine carrier can be used to express a Chlamydia HtrA polypeptide. Such viral vaccine carriers include, but are not limited to modified vaccinia virus (e.g., MVA).

Alternatively, Chlamydia HtrA polypeptide vaccine antigens can be purified from infected-host cell culture. For instance, if a baculovirus expression system is used to express a Chlamydia HtrA polypeptide, the polypeptide can be isolated and purified from a baculovirus infected insect cell culture. Accordingly, the present invention should be understood to include a Chlamydia HtrA polypeptide expressed in insect cells using a baculovirus expression system.

A suitable expression vector contains regulatory sequences which can be operably joined to an inserted nucleotide sequence encoding a C. trachomatis HtrA polypeptide vaccine antigen. As used herein, the term “regulatory sequences” means nucleotide sequences which are necessary for or conducive to the transcription of an inserted sequence coding a C. trachomatis HtrA vaccine antigen by a host cell and/or which are necessary for or conducive to the translation by a host cell of the resulting transcript into the desired HtrA polypeptide. Regulatory sequences include, but are not limited to, 5′ sequences such as operators, promoters and ribosome binding sequences, and 3′ sequences such as polyadenylation signals. Regulatory sequences may also include enhancer sequences or upstream activator sequences.

Generally, bacterial vectors will include origins of replication and selectable markers, e.g., the ampicillin, tetracycline, kanamycin, resistance genes of E. coli, permitting transformation of the host cell and a promoter derived from a highly-expressed gene to direct transcription of a downstream structural sequence. Such promoters include, but are not limited to, the T7 promoter, lambda (λ) promoter, T5 promoter, and lac promoter, or promoters derived from operons encoding glycolytic enzymes such as 3-phosphoglycerate kinase (PGK), acid phosphatase, or heat shock proteins, among others.

Once an expression vector is selected, the polynucleotide of the invention is cloned downstream of the promoter, often in a polylinker region. This plasmid is transformed into an appropriate bacterial strain, and DNA is prepared using standard techniques. The orientation and DNA sequence of the polynucleotide as well as all other elements included in the vector, are confirmed using restriction mapping, DNA sequence analysis, and/or PCR analysis. Bacterial cells harboring the correct plasmid can be stored as cell banks.

Examples of mammalian host-expression systems include cell lines capable of expressing a compatible vector, for example, the COS, C127, 3T3, CHO, HeLa and BHK cell lines. Examples of suitable expression vectors include pWLNEO, pSV2CAT, pOG44, pXT1, pSG (Stratagene) pSVK3, pBPV, pMSG, pSVL (Pharmacia), p75.6 (Valentis), pCEP (Invitrogen), pCEI (Epimmune), pZERO, pTrc99A, pUC19, pUC18, pKK223-3, pEX1, pCAL, pET, pSPUTK, pTrxFus, pFastBac, pThioHis, pTrcHis, and pLEx, pET-17b, pET-11a, pET-24a-d(+) and pET-9a pK233 (or any of the tac family of plasmids), pT7, lambda pSKF, and pET-28(a)+, vectors useful in yeast cells, including YIp, YRp, YCP, YEp and YLp plasmids as well as viral genomes from which to construct viral vectors such as Simian virus 40 (SV40), bovine papilloma virus, pox virus such as vaccinia virus, e.g., VV MVA, and parvovirus, including adeno-associated virus, retrovirus, herpesvirus, adenovirus, retroviral, e.g., murine leukemia virus and lentiviruses (e.g., human immunodeficiency virus), alphavirus, and picornavirus. References citing methods for the in vivo introduction of non-infectious virus genomes to animal tissues are well known to those of ordinary skill in the art. Any of a variety of methods known in the art can be used to insert a nucleotide sequence coding for a C. trachomatis HtrA polypeptide vaccine antigen into a suitable expression vector.

Generally, mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters may also be derived from viral sources, such as, e.g., human cytomegalovirus (CMV-IE promoter), herpes simplex virus type-1 (HSV TK promoter), the adenovirus late promoter; and the vaccinia virus 7.5K promoter, or can be derived from the genome of mammalian cells (e.g., metallothionein promoter). Nucleic acid sequences derived from the SV40 splice and polyadenylation sites can be used to provide the required nontranscribed genetic elements. A variety of transcription control regions are known to those skilled in the art. These include, without limitation, transcription control regions which function in animal cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from animal genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins). Similarly, a variety of translation control elements are known to those of ordinary skill in the art. These include, but are not limited to ribosome binding sites, translation initiation and termination codons, elements from picornaviruses (particularly an internal ribosome entry site, or IRES, also referred to as a CITE sequence).

Yeast host-expression systems include a yeast host (e.g., Saccharomyces, Pichia, Hansenula, Kluyveromyces, Schizosaccharomyces, Schwanniomyces and Yarrowia) transformed with recombinant yeast expression vectors containing polypeptide coding sequences, employing suitable vectors and control sequences. Suitable yeast expression vectors are known to those in the art and include, but are not limited to, e.g., pAL19, paR3, pBG1, pDBlet, pDB248X, pEA500, pFL20, pIRT2, pJK148, pON163, pSP1, pSP3, pUR19, pART1, pCHY21, REP41, pYZ1N, pSLF104, pSLF172, pDS472, pSGP572, pSLF1072, REP41 MH-N, pFA6a-kanMX6, pARTCM, and pALL.

Insect host systems (e.g., Trichoplusia, Lipidotera, Spodoptera, Drosophila and Sf9) infected with recombinant expression vectors (e.g., baculovirus, pDEST™10 Vector (Invitrogen), pMT-DEST48 Vector (Invitrogen), pFastBac Dual (Invitrogen), pIE1-neo DNA (Novagen), pIEX™-1 DNA (Novagen), containing polypeptide coding sequences of the present invention are also within the scope of the invention. See e.g., O'Reilly et al., Baculovirus Expression Vectors: A Laboratory Manual. Oxford Univ Press (1994).

Plant cell systems (e.g., Arabidopsis) infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences of the present invention, containing polypeptide coding sequences are also within the scope of the invention. A list of vectors for a wide variety of plants can be found at http://www.arabidopsis.org/servlets/Order?state=catalog (viewed Jun. 20, 2006).

One of skill in the art will recognize that some of the above listed vectors are capable of replicating and expressing polypeptides in more than one type of host, e.g., the pOG44 plasmid can replicate and express polypeptides in both prokaryotic and eukaryotic cells.

### Polypeptides

Also included within the scope of the invention are full-length or mature C. trachomatis HtrA polypeptides or fragments of a full-length or mature C. trachomatis HtrA polypeptide, or of serotypic or other variants, analogs, or derivatives of C. trachomatis HtrA.

The present invention includes the C. trachomatis HtrA polypeptide of all serotypes including, but not limited to, A, B, Ba, C, D, E, F, G, H, I, J, K, L₁, L₂, L₂ₐ, L₃, or MoPn. The invention also includes HtrA polypeptide from Chlamydia pneumoniae. C. trachomatis is comprised of two human biovars: the trachoma biovar and lymphogranuloma venereum (LGV) biovar. The LGV biovar consists of four serovars, L₁, L₂, L₂ₐ, and L₃. Serovars in both C. trachomatis biovars cause trachoma, sexually transmitted disease, some forms of arthritis, and neonatal inclusion conjunctivitis and pneumonia.

In certain embodiments, the present invention is directed to a fragment which comprises one or more CD4(+) Th1 epitopes. CD4(+) Th1 epitopes are formed from contiguous amino acid residues and comprise at least 10 and typically 12-25 consecutive amino acid residues. (Watts, Nature Immun., 5(7):685-692, 2004; Sercarz, et al., Nature Reviews Immun., 3:621-629, 2003). In addition, B-cell epitopes typically comprise 8-10 amino acid residues in a distinct spatial arrangement, and can be formed from either contiguous amino acid residues, or from residues which are separated in primary sequence but spatially related in the folded protein. Epitopic fragments of SEQ ID NO: 2, SEQ ID NO: 4, SEQ ID NO: 6, and SEQ ID NO: 8 can be readily identified using techniques of epitope mapping well known in the art. See, e.g., Zhang, et al., Proc Natl Acad Sci USA, 85(11):4000-4, 1988; Conlan, et al., Mol Microbio., 2(5):673-9, 1988; and Stephens, et al., Exp Med., 167(3):817-31, 1988. (Carmicle, S., et al., J. Biol. Chem., 227(1):155-160, 2002)

Immunogenic fragments of Chlamydia, for instance, C. trachomatis, HtrA polypeptides useful in vaccine compositions may consist of at least 7, 8, 10, 12, 15, 20, 30, 33, 60, 80, 100, 115, 130, 160, 190, 200, 230, 260, 290, 320, 350, 380, 410 or 440 consecutive amino acid residues of the sequence shown in SEQ ID NOs: 2, 6, or 8. These fragments may be produced by recombinant expression of a nucleic acid consisting of at least 21, 24, 30, 33, 36, 45, 60, 90, 99, 180, 240, 300, 345, 390, 450, 480, 570, 600, 690, 780, 870, 960, 1050, 1140, 1230, or 1320 consecutive nucleotides of the sequence shown in SEQ ID NOs: 1, 5, or 7.

The present invention is also directed to a polypeptide at least 70%, 75%, 80%, 85%, 90%, 94%, 95%, 96%, 97%, 98%, 99%, or 100% identical to a full-length or mature Chlamydia, for instance, C. trachomatis or C. pneumoniae, HtrA polypeptide, or fragments, analogs, variants, or derivatives thereof. In particular, the present invention is directed to a polypeptide comprising an amino acid sequence at least 70%, 75%, 80%, 85%, 90%, 94%, 95%, 96%, 97%, 98%, 99%, or 100% % identical to a reference amino acid sequence selected from the group consisting of: amino acids a-483 of SEQ ID NO: 2; amino acids 22-b of SEQ ID NO: 2; amino acids a-b of SEQ ID NO: 2; amino acids c-379 of SEQ ID NO: 2; amino acids 300-d of SEQ ID NO: 2; amino acids c-d of SEQ ID NO: 2; amino acids e-483 of SEQ ID NO: 2; amino acids 428-b of SEQ ID NO: 2; amino acids e-b of SEQ ID NO: 2; amino acids c-483 of SEQ ID NO: 2; amino acids 300-b of SEQ ID NO: 2; amino acids c-b of SEQ ID NO: 2; amino acids f-288 of SEQ ID NO: 2; amino acids 128-g of SEQ ID NO: 2; amino acids f-g of SEQ ID NO: 2; amino acids f-379 of SEQ ID NO: 2; amino acids 128-d of SEQ ID NO: 2; amino acids f-d of SEQ ID NO: 2; amino acids f-483 of SEQ ID NO: 2; amino acids 128-b of SEQ ID NO: 2; amino acids f-b of SEQ ID NO: 2; amino acids a-379 of SEQ ID NO: 2; amino acids 22-d of SEQ ID NO: 2; amino acids a-d of SEQ ID NO: 2; amino acids a-288 of SEQ ID NO: 2; amino acids 22-g of SEQ ID NO: 2; and amino acids a-g of SEQ ID NO: 2; wherein a is any integer between 17 and 22, b is any integer between 483-497, c is any integer between 290-300, d is any integer between 379-389, e is any integer between 394-428, f is any integer between 95-128, and g is any integer between 288 and 297. In another embodiment, the present invention is directed to the polypeptide, which lacks an amino acid sequence selected from the group consisting of: amino acids f-288 of SEQ ID NO: 2; amino acids 128-g of SEQ ID NO: 2; amino acids f-g of SEQ ID NO: 2; amino acids c-379 of SEQ ID NO: 2; amino acids 300-d of SEQ ID NO: 2; and amino acids c-d of SEQ ID NO: 2, wherein c is any integer between 290-300, d is any integer between 379-389, f is any integer between 95-128, and g is any integer between 288-297. The polypeptide is not SEQ ID NOs: 6 or 8.

For example, the reference amino acid sequence is selected from the group consisting of: amino acids 22-483 of SEQ ID NO: 2; amino acids 17-483 of SEQ ID NO: 2; amino acids 22-497 of SEQ ID NO: 2; amino acids 17-497 of SEQ ID NO: 2; amino acids 290-381 of SEQ ID NO: 2; amino acids 290-379 of SEQ ID NO: 2; amino acids 300-382 of SEQ ID NO: 2; amino acids 291-381 of SEQ ID NO: 2; amino acids 300-389 of SEQ ID NO: 2; amino acids 394-485 of SEQ ID NO: 2; amino acids 428-483 of SEQ ID NO: 2; amino acids 404-486 of SEQ ID NO: 2; amino acids 407-485 of SEQ ID NO: 2; amino acids 404-493 of SEQ ID NO: 2; amino acids 290-485 of SEQ ID NO: 2; amino acids 290-483 of SEQ ID NO: 2; amino acids 300-486 of SEQ ID NO: 2; amino acids 291-485 of SEQ ID NO: 2; amino acids 300-493 of SEQ ID NO: 2; amino acids 128-381 of SEQ ID NO: 2; amino acids 128-379 of SEQ ID NO: 2; amino acids 110-382 of SEQ ID NO: 2; amino acids 95-389 of SEQ ID NO: 2; amino acids 128-485 of SEQ ID NO: 2; amino acids 128-483 of SEQ ID NO: 2; amino acids 110-486 of SEQ ID NO: 2; and amino acids 95-493 of SEQ ID NO: 2.

The present invention further provides a polypeptide comprising an amino acid sequence encoded by a polynucleotide described herein or a combination of one or more of the amino acid sequence, provided that the amino acid sequence is not SEQ ID NOs: 6, or 8, wherein said polypeptide when administered to a subject in need thereof in a sufficient amount, induces an immune response against Chlamydia sp.

The present invention provides a polypeptide comprising, consisting essentially of, or consisting of an amino acid sequence at least 70%, 75%, 80%, 85%, 90%, 95%, 96%, 97%, 98%, 99%, or 100% identical to amino acids 327-497 of SEQ ID NO: 2, amino acids 437-497 of SEQ ID NO: 2, or amino acids 327-436 of SEQ ID NO: 2, provided that the amino acid sequence is not SEQ ID NO: 6 or SEQ ID NO: 8, wherein the polypeptide when administered to a subject in need thereof in a sufficient amount, induces an immune response against Chlamydia sp. The present invention also includes a polypeptide encoded by a polynucleotide comprising, consisting essentially of, or consisting of a nucleic acid sequence at least 60%, 70%, 75%, 80%, 85%, 90%, 95%, 96%, 97%, 98%, 99%, or 100% identical to nucleotide residues 979-1491 of SEQ ID NO: 1, nucleotide residues 1307-1491 of SEQ ID NO: 1, or nucleotide residues 976-1308 of SEQ ID NO: 1, provided that the polypeptide is not SEQ ID NO: 6, or SEQ ID NO: 8, wherein the polynucleotide encodes the polypeptide that can induce an immune response against Chlamydia sp. when administered to a subject in need thereof in a sufficient amount.

The invention further provides any combinations of fragments produced by Cathepsin S cleavage sites shown in Table 4. For example, the present invention is directed to a polypeptide comprising, consisting essentially of, or consisting of an amino acid sequence at least 70%, 75%, 80%, 85%, 90%, 95%, 96%, 97%, 98%, 99%, or 100% identical to amino acids 1-19, amino acids 1-79, amino acids 1-112, amino acids 1-187, amino acids 1-326, amino acids 1-436, amino acids 20-79, amino acids 20-112, amino acids 20-187, amino acids 20-326, amino acids 20-436, amino acids 20-497, amino acids 80-112, amino acids 80-187, amino acids 80-326, amino acids 80-436, amino acids 80-497, amino acids 113-187, amino acids 113-326, amino acids 113-436, amino acids 113-497, amino acids 188-326, amino acids 188-436, amino acids 188-497, amino acids 327-436, amino acids 327-497, amino acids 437-497 of SEQ ID NO: 2 or two or more such amino acid sequences in combination, provided that the amino acid sequence is not SEQ ID NOs: 6, or 8, wherein the polypeptide when administered to a subject in need thereof in a sufficient amount, induces an immune response against Chlamydia sp.

In other embodiments, the present invention includes a polypeptide comprising, consisting essentially of, or consisting of an amino acid sequence at least 70%, 75%, 80%, 85%, 90%, 95%, 96%, 97%, 98%, 99%, or 100% identical to amino acids 69-497 of SEQ ID NO: 2, amino acids 145-497 of SEQ ID NO: 2, amino acids 219-497 of SEQ ID NO: 2, or amino acids 327-497 of SEQ ID NO: 2, provided that the amino acid sequence is not SEQ ID NOs: 6 or 8, wherein the polypeptide when administered to a subject in need thereof in a sufficient amount, induces an immune response against Chlamydia sp. The present invention also includes a polypeptide comprising, consisting essentially of, or consisting of an amino acid sequence encoded by a nucleic acid sequence at least 60%, 70%, 75%, 80%, 85%, 90%, 95%, 96%, 97%, 98%, 99%, or 100% identical to nucleotide residues 205-1491 of SEQ ID NO: 1, nucleotide residues 433-1491 of SEQ ID NO: 1, or nucleotide residues 655-1491 of SEQ ID NO: 1, provided that the amino acid sequence encoded by the nucleotide sequence is not SEQ ID NO: 6 or SEQ ID NO: 8, wherein the polynucleotide encodes a polypeptide that can induce an immune response against Chlamydia sp. when administered to a subject in need thereof in a sufficient amount.

Further included is a polypeptide comprising, consisting essentially of, or consisting of an amino acid sequence at least 70%, 75%, 80%, 85%, 90%, 94%, 95%, 96%, 97%, 98%, 99%, or 100% identical to a reference amino acid sequence selected from the group consisting of amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ID NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 30; and a combination of at least two of any of said polypeptide fragments or variants or derivatives thereof, provided that said amino acid sequence is not SEQ ID NOs: 6, or 8, wherein said polypeptide when administered to a subject in need thereof in a sufficient amount, induces an immune response against Chlamydia sp.

The present invention further includes a polypeptide comprising, consisting essentially of, or consisting of an amino acid sequence encoded by a nucleic acid sequence at least 60%, 70%, 75%, 80%, 85%, 90%, 95%, 96%, 97%, 98%, 99%, or 100% identical to the corresponding nucleotide residues of SEQ ID NO: 1 or fragments, variants, analogs, or derivatives thereof, provided that the amino acid sequence encoded by the nucleic acid sequence is not SEQ ID NOs: 6 or 8, wherein the polypeptide encoded by said nucleic acid sequence induces an immune response against Chlamydia sp. when administered to a subject in need thereof in a sufficient amount. A person of ordinary skill in the art can readily calculate the exact coordinate of the nucleotide residues of SEQ ID NO: 1 encoding fragments of SEQ ID NO: 2.

In some embodiments, the present invention is further directed to a polypeptide comprising, consisting essentially of, or consisting of an amino acid sequence selected from a group consisting of amino acids 1-19 of SEQ ID NOs:2, 6, or 8; amino acids 1-68 of SEQ ID NOs:2, 6, or 8, amino acids 1-79 of SEQ ID NOs:2, 6, or 8; amino acids 1-112 of SEQ ID NOs:2, 6, or 8; amino acids 1-144 of SEQ ID NOs:2, 6, or 8; amino acids 1-218 of SEQ ID NOs:2, 6, or 8; amino acids 1-299 of SEQ ID NOs:2, 6, or 8; amino acids 1-326 of SEQ ID NOs:2, 6, or 8; amino acids 1-392 of SEQ ID NOs:2, 6, or 8; amino acids 1-436 of SEQ ID NOs:2, 6, or 8; amino acids 1-449 of SEQ ID NOs:2, 6, or 8; amino acids 17-68 of SEQ ID NOs:2, 6, or 8; amino acids 17-79 of SEQ ID NOs:2, 6, or 8; amino acids 17-112 of SEQ ID NOs:2, 6, or 8; amino acids 17-144 of SEQ ID NOs:2, 6, or 8; amino acids 17-218 of SEQ ID NOs:2, 6, or 8; amino acids 17-299 of SEQ ID NOs:2, 6, or 8; amino acids 17-326 of SEQ ID NOs:2, 6, or 8; amino acids 17-392 of SEQ ID NOs:2, 6, or 8; amino acids 17-436 of SEQ ID NOs:2, 6, or 8; amino acids 17-449 of SEQ ID NOs:2, 6, or 8; amino acids 20-68 of SEQ ID NOs:2, 6, or 8; amino acids 20-79 of SEQ ID NOs:2, 6, or 8; amino acids 20-112 of SEQ ID NOs:2, 6, or 8; amino acids 20-144 of SEQ ID NOs:2, 6, or 8; amino acids 20-218 of SEQ ID NOs:2, 6, or 8; amino acids 20-299 of SEQ ID NOs:2, 6, or 8; amino acids 20-326 of SEQ ID NOs:2, 6, or 8; amino acids 20-392 of SEQ ID NOs:2, 6, or 8; amino acids 20-436 of SEQ ID NOs:2, 6, or 8; amino acids 20-449 of SEQ ID NOs:2, 6, or 8; amino acids 20-497 of SEQ ID NOs:2, 6, or 8; amino acids 69-112 of SEQ ID NOs:2, 6, or 8; amino acids 69-144 of SEQ ID NOs:2, 6, or 8; amino acids 69-218 of SEQ ID NOs:2, 6, or 8; amino acids 69-299 of SEQ ID NOs:2, 6, or 8; amino acids 69-326 of SEQ ID NOs:2, 6, or 8; amino acids 69-392 of SEQ ID NOs:2, 6, or 8; amino acids 69-436 of SEQ ID NOs:2, 6, or 8; amino acids 69-449 of SEQ ID NOs:2, 6, or 8; amino acids 69-497 of SEQ ID NOs:2, 6, or 8; amino acids 80-112 of SEQ ID NOs:2, 6, or 8; amino acids 80-144 of SEQ ID NOs:2, 6, or 8; amino acids 80-218 of SEQ ID NOs:2, 6, or 8; amino acids 80-299 of SEQ ID NOs:2, 6, or 8; amino acids 80-326 of SEQ ID NOs:2, 6, or 8; amino acids 80-392 of SEQ ID NOs:2, 6, or 8; amino acids 80-436 of SEQ ID NOs:2, 6, or 8; amino acids 80-449 of SEQ ID NOs:2, 6, or 8; amino acids 80-497 of SEQ ID NOs:2, 6, or 8; amino acids 113-144 of SEQ ID NOs:2, 6, or 8; amino acids 113-218 of SEQ ID NOs:2, 6, or 8; amino acids 113-299 of SEQ ID NOs:2, 6, or 8; amino acids 113-326 of SEQ ID NOs:2, 6, or 8; amino acids 113-392 of SEQ ID NOs:2, 6, or 8; amino acids 113-436 of SEQ ID NOs:2, 6, or 8; amino acids 113-449 of SEQ ID NOs:2, 6, or 8; amino acids 113-497 of SEQ ID NOs:2, 6, or 8; amino acids 145-218 of SEQ ID NOs:2, 6, or 8; amino acids 145-299 of SEQ ID NOs:2, 6, or 8; amino acids 145-326 of SEQ ID NOs:2, 6, or 8; amino acids 145-392 of SEQ ID NOs:2, 6, or 8; amino acids 145-436 of SEQ ID NOs:2, 6, or 8; amino acids 145-449 of SEQ ID NOs:2, 6, or 8; amino acids 145-497 of SEQ ID NOs:2, 6, or 8; amino acids 219-299 of SEQ ID NOs:2, 6, or 8; amino acids 219-326 of SEQ ID NOs:2, 6, or 8; amino acids 219-392 of SEQ ID NOs:2, 6, or 8; amino acids 219-436 of SEQ ID NOs:2, 6, or 8; amino acids 219-449 of SEQ ID NOs:2, 6, or 8; amino acids 219-497 of SEQ ID NOs:2, 6, or 8; amino acids 300-392 of SEQ ID NOs:2, 6, or 8; amino acids 300-436 of SEQ ID NOs:2, 6, or 8; amino acids 300-449 of SEQ ID NOs:2, 6, or 8; amino acids 300-497 of SEQ ID NOs:2, 6, or 8; amino acids 327-392 of SEQ ID NOs:2, 6, or 8; amino acids 327-436 of SEQ ID NOs:2, 6, or 8; amino acids 327-449 of SEQ ID NOs:2, 6, or 8; amino acids 327-497 of SEQ ID NOs:2, 6, or 8; amino acids 393-436 of SEQ ID NOs:2, 6, or 8; amino acids 393-449 of SEQ ID NOs:2, 6, or 8; amino acids 393-497 of SEQ ID NOs:2, 6, or 8; amino acids 437-497 of SEQ ID NOs:2, 6, or 8; amino acids 450-497 of SEQ ID NOs:2, 6, or 8; and a combination of at least two of any of said polypeptide fragments or variants or derivatives thereof, provided that the amino acid sequence is not SEQ ID NOs: 6 or 8, wherein said polypeptide when administered to a subject in need thereof in a sufficient amount, induces an immune response against Chlamydia sp.

In other embodiments, the present invention is directed to any C. trachomatis HtrA polypeptides described herein, or fragments, derivatives, or variants thereof which are encoded by codon-optimized polynucleotides. For example, in certain embodiments, the polypeptide is encoded by a human codon-optimized coding region and is expressed in human cells, e.g., via a viral vector. In addition, a polypeptide comprising an amino acid sequence at least 70%, 75%, 80%, 85%, 90%, 91%, 92%, 93%, 94%, 95%, 96%, 97%, 98%, 99%, or 100% identical to SEQ ID NO:2, or fragments, derivatives, or variants thereof, wherein the amino acid sequence is encoded by an E. coli or P. fluorescens codon optimized coding region, are also within the scope of the invention.

In other embodiments, the present invention includes a polypeptide fused with a heterologous polypeptide. The heterologous polypeptide can be translated from various heterologous nucleic acids. Various heterologous polypeptides can be used, and can be selected from the group consisting of an N- or C-terminal peptide imparting stabilization, secretion, or simplified purification, i.e., His-tag (SEQ ID NO: 4), ubiquitin tag, NusA tag, chitin binding domain, ompT, ompA, pelB, DsbA, DsbC, c-myc, KSI, polyaspartic acid, (Ala-Trp-Trp-Pro)n, polyphenyalanine, polycysteine, polyarginine, B-tag, HSB-tag, green fluorescent protein (GFP), hemagglutinin influenza virus (HAI), calmodulin binding protein (CBP), galactose-binding protein, maltose binding protein (MBP), cellulose binding domains (CBD's), dihydrofolate reductase (DHFR), glutathione-S-transferase (GST), streptococcal protein G, staphylococcal protein A, T7gene10, avidin/streptavidin/Strep-tag, trpE, chloramphenicol acetyltransferase, lacZ (β-Galactosidase), His-patch thioredoxin, thioredoxin, FLAG™ peptide (Sigma-Aldrich), S-tag, and T7-tag. See e.g., Stevens, R. C., Structure, 8:R177-R185 (2000). The heterologous polypeptides can further include any pre- and/or pro-sequences that facilitate the transport, translocations, processing and/or purification of C. trachomatis HtrA vaccine antigens from a host cell or any useful immunogenic sequence, including but not limited to sequences that encode a T-cell epitope of a microbial pathogen, or other immunogenic proteins and/or epitopes.

Other Chlamydia proteins (either native proteins or variants, fragments, or derivatives thereof, e.g., MOMP, PorB, Pmp6, Pmp8, Pmp11, Pmp20, Pmp21, PmpD, PmpE, PmpG, PmpH, PmpI, OmpH, Omp4, Omp5, Omp85, MIP, OmcA, and OmcB) may be fused to a Chlamydia HtrA polypeptide of the present invention. For instance, the heterologous polypeptide may comprise a PmpG polypeptide fragment such as CT84 as described in U.S. Application 60/929,129, filed Jun. 14, 2008, which is herein incorporated by reference in its entirety. Accordingly, the present invention includes a HtrA and PmpG fusion protein. In some embodiments, the fusion protein may further comprise a leader sequence capable of directing secretion of translated protein into the periplasmic space or extracellular medium.

In some embodiments, the polypeptide of the present invention can exist as a homopolymer, comprising multiple copies of the same polypeptide.

Optionally, a HtrA polypeptide fused with a heterologous polypeptide or other Chlamydia polypeptide can include a peptide linker sequence joining sequences that comprise two or more epitopes. Suitable peptide linker sequences may be chosen based on their ability to adopt a flexible, extended conformation, or a secondary structure that could interact with joined epitopes, or based on their ability to increase overall solubility of the fusion polypeptide, or based on their lack of electrostatic or water-interaction effects that influence joined epitopes. An example is a polypeptide comprising the sequence of SEQ ID NO: 30. This comprises two “copies” of the T-cell epitope of SEQ ID NO: 19, joined with the flexible linker GGSGGS, described by Wang, et al., Haematologica, 87(10): 1087-1094, 2002.

Peptide and polypeptide sequences defined herein are represented by one-letter symbols for amino acid residues as follows: A (alanine); R (arginine); N (asparagine); D (aspartic acid); C (cysteine); Q (glutamine); E (glutamic acid); G (glycine); H (histidine); I (isoleucine); L (leucine); K (lysine); M (methionine); F (phenylalanine); P (proline); S (serine); T (threonine); W (tryptophan); Y (tyrosine); and V (valine).

In some embodiments, the polypeptides of the present invention are isolated. No particular level of purification is required. Recombinantly produced Chlamydia polypeptides and proteins expressed in host cells are considered isolated for purposes of the invention, as are native or recombinant Chlamydia polypeptides which have been separated, fractionated, or partially or substantially purified by any suitable technique, including by filtration, chromatography, centrifugation, and the like.

Polypeptides, and fragments, derivatives, analogs, or variants thereof of the present invention can be antigenic and immunogenic Chlamydia polypeptides, which are used to prevent or treat, i.e., cure, ameliorate, lessen the severity of, or prevent or reduce contagion of infectious disease caused by C. trachomatis, or other species as disclosed herein. The term “antigens” and its related term “antigenic” as used herein and in the claims refer to a substance that binds specifically to an antibody or T-cell receptor. In some embodiments the antigens are immunogenic. The term “immunogen,” as used herein, refers to an antigen capable of inducing an immune response directed against itself when administered to a subject.

In certain aspects of the present invention, antigenic epitopes can contain a sequence of at least 4, at least 5, at least 6, at least 7, at least 8, at least 9, at least 10, at least 15, at least 20, at least 25, or between about 15 to about 30 amino acids contained within the amino acid sequence of a polypeptide of the invention. Certain polypeptides comprising immunogenic or antigenic epitopes are at least 10, 15, 20, 25, 30, 35, 40, 45, 50, 55, 60, 65, 70, 75, 80, 85, 90, 95, or 100 or more amino acid residues in length. Antigenic as well as immunogenic epitopes may be linear, i.e., be comprised of contiguous amino acids in a polypeptide, or may be three dimensional, i.e., where an epitope is comprised of non-contiguous amino acids which come together due to the secondary or tertiary structure of the polypeptide, thereby forming an epitope.

Peptides or polypeptides, e.g., immunogenic epitopes, capable of eliciting an immunogenic response are frequently represented in the primary sequence of a protein, can be characterized by a set of simple chemical rules, and are confined neither to immunodominant regions of intact proteins nor to the amino or carboxyl terminals. Polypeptides that are extremely hydrophobic and those of six or fewer residues generally are ineffective at inducing antibodies, but may still bind antibodies raised against larger portions of the polypeptide; longer peptides, especially those containing proline residues, usually are effective (Sutcliffe, J. G., et al., Science 219:660-666 (1983)).

Production of the polypeptides of the present invention can be achieved by culturing the host cells, expressing the polynucleotides of the present invention, and recovering the polypeptides. Determining conditions for culturing the host cells and expressing the polynucleotide are generally specific to the host cell and the expression system and are within the knowledge of one of skill in the art. Likewise, appropriate methods for recovering the polypeptide of interest are known to those in the art, and include, but are not limited to, chromatography, filtration, precipitation, or centrifugation.

### Compositions

Compositions, e.g., vaccine compositions, that contain an immunologically effective amount of one or more polypeptides or polynucleotides of the invention are a further embodiment of the invention. Such compositions may include, for example, lipopeptides (e.g., Vitiello, A. et al., J. Clin. Invest. 95:341, 1995), polypeptides encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995); polypeptide compositions contained in immune stimulating complexes (ISCOMS) (see, e.g., Takahashi et al., Nature 344:873-875, 1990; Hu, et al., Clin Exp Immunol. 113:235-243, 1998); multiple antigen peptide systems (MAPs) (see e.g., Tam, J. P., Proc. Natl. Acad. Sci. U.S.A. 85:5409-5413, 1988; Tam, J. P., J. Immunol. Methods 196:17-32, 1996); particles of viral or synthetic origin (e.g., Kofler, N. et al., J. Immunol. Methods. 192:25, 1996; Eldridge, J. H. et al., Sem. Hematol. 30:16, 1993; Falo, L. D., Jr. et al., Nature Med. 7:649, 1995); adjuvants (e.g., incomplete Freund's adjuvant) (Warren, H. S., Vogel, F. R., and Chedid, L. A. Annu. Rev. Immunol. 4:369, 1986; Gupta, R. K. et al., Vaccine 11:293, 1993); or liposomes (Reddy, R. et al., J. Immunol. 148:1585, 1992; Rock, K. L., Immunol. Today 17:131, 1996). The compositions can be pharmaceutical, antigenic, immunogenic, or vaccine compositions.

Compositions, e.g, vaccine compositions, of the present invention can be formulated according to known methods. Suitable preparation methods are described, for example, in Remington's Pharmaceutical Sciences, 16th Edition, A. Osol, ed., Mack Publishing Co., Easton, Pa. (1980), and Remington's Pharmaceutical Sciences, 19th Edition, A. R. Gennaro, ed., Mack Publishing Co., Easton, Pa. (1995), both of which are incorporated herein by reference in their entireties. Although the composition may be administered as an aqueous solution, it can also be formulated as an emulsion, gel, solution, suspension, lyophilized form, or any other form known in the art. In addition, the composition may contain pharmaceutically acceptable additives including, for example, diluents, binders, stabilizers, and preservatives. Once formulated, the compositions of the invention can be administered directly to the subject. The subjects to be treated can be animals; in particular, human subjects can be treated.

The concentration of polypeptides of the invention in the compositions of the invention can vary widely, i.e., from less than about 0.1%, usually at or at least about 2% to as much as 20% to 50% or more by weight, and will be selected primarily by fluid volumes, viscosities, etc., in accordance with the particular mode of administration selected.

Furthermore, compositions in accordance with the invention can comprise more than one polypeptide. In some embodiments, a composition, e.g., a vaccine composition of the present invention can further include a polypeptide selected from the group consisting of, but not limited to, MOMP, PorB, Pmp6, Pmp8, Pmp11, Pmp20, Pmp21, PmpD, PmpE, PmpG, PmpH, PmpI, OmpH, Omp4, Omp5, Omp85, MIP, OmcA, and OmcB; as well as more than one HtrA polypeptide described herein.

In other embodiments, the present invention includes a composition comprising a polypeptide fused with a heterologous polypeptide described herein. For example, in certain embodiments, a composition, e.g., a vaccine composition of the present invention comprises a polypeptide having the sequence of SEQ ID NO: 30. SEQ ID NO: 30 is an example of a Chlamydia HtrA polypeptide, i.e., amino acids 450-497 of SEQ ID NO: 2, fused to one or more a heterologous polypeptides.

In some embodiments, a host cell having a vector expressing the polypeptide of the present invention is incorporated in a composition, as described in Eko, et al., J. Immunol., 173:3375-3382, 2004.

The present invention is also directed to a method of producing a polypeptide vaccine composition effective against Chlamydia infection. In some embodiments, the method of producing the composition comprises (a) isolating the polypeptide of the present invention; and (b) adding an adjuvant, carrier and/or excipient to the isolated polypeptide. As the person of ordinary skill in the art would appreciate, the terms “adjuvant,” “carrier,” and “excipient” overlap to a significant extent. For example, a compound which acts as an “adjuvant” may also be a “carrier,” as well as an “excipient,” and certain other compounds normally thought of, e.g., as carriers, may also function as an adjuvant. The term “adjuvant” refers to any material having the ability to (1) alter or increase the immune response to a particular antigen or (2) increase or aid an effect of a pharmacological agent. Any compound which may increase the expression, antigenicity or immunogenicity of the polypeptide is a potential adjuvant. The term “immunogenic carrier” as used herein refers to a first polypeptide or fragment, variant, or derivative thereof which enhances the immunogenicity of a second polypeptide or fragment, variant, or derivative thereof.

In some embodiments, the present invention provides a composition comprising a polypeptide at least 70%, 75%, 80%, 85%, 90%, 95%, 96%, 97%, 98%, 99%, or 100% identical to any Chlamydia, for instance C. trachomatis or C. pneumoniae, HtrA sequences, fragments, analogs, derivatives, or variants described herein, e.g., in Tables 2-8 and a carrier. Carriers that can be used with compositions of the invention are well known in the art, and include, without limitation, e.g., thyroglobulin, albumins such as human serum albumin, tetanus toxoid, polyamino acids such as poly L-lysine, poly L-glutamic acid, influenza, hepatitis B virus core protein, and the like. A variety of aqueous carriers may be used, e.g., water, buffered water, 0.8% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the carrier is an immunogenic carrier. Suitably, an immunogenic carrier may be fused to or conjugated to a desired polypeptide or fragment thereof. See, e.g., European Patent No. EP 0385610 B1, which is incorporated herein by reference in its entirety.

Certain compositions can further include one or more adjuvants before, after, or concurrently with the polypeptide. A great variety of materials have been shown to have adjuvant activity through a variety of mechanisms. Potential adjuvants which may be screened for their ability to enhance the immune response according to the present invention include, but are not limited to: inert carriers, such as alum, bentonite, latex, and acrylic particles; pluronic block polymers, such as TITERMAX® (block copolymer CRL-8941, squalene (a metabolizable oil) and a microparticulate silica stabilizer), depot formers, such as Freund's adjuvant, surface active materials, such as saponin, lysolecithin, retinal, Quil A, liposomes, and pluronic polymer formulations; macrophage stimulators, such as bacterial lipopolysaccharide; polycationic polymers such as chitosan; alternate pathway complement activators, such as insulin, zymosan, endotoxin, and levamisole; and non-ionic surfactants, such as poloxamers, poly(oxyethylene)-poly(oxypropylene) tri-block copolymers, cytokines and growth factors; bacterial components (e.g., endotoxins, in particular superantigens, exotoxins and cell wall components); aluminum-based salts such as aluminum hydroxide; calcium-based salts; silica; polynucleotides; toxoids; serum proteins, viruses and virally-derived materials, poisons, venoms, imidazoquiniline compounds, poloxamers, mLT, and cationic lipids. International Patent Application, PCT/US95/09005 incorporated herein by reference describes use of a mutated form of heat labile toxin of enterotoxigenic E. coli (“mLT”) as an adjuvant. U.S. Pat. No. 5,057,540, incorporated herein by reference, describes the adjuvant, Qs21. In some embodiments, the adjuvant is a toll-like receptor (TLR) stimulating adjuvant. See e.g., Science 312:184-187 (2006). TLR adjuvants include compounds that stimulate the TLRs (e.g., TLR1-TLR17), resulting in an increased immune system response to the vaccine composition of the present invention. TLR adjuvants include, but are not limited to CpG (Coley Pharmaceutical Group Inc.) and MPL (Corixa). One example of a CpG adjuvant is CpG7909, described in WO 98/018810, US Patent Application Publication. No. 2002/0164341 A, U.S. Pat. No. 6,727,230, and WO98/32462, which are incorporated herein by reference in their entireties.

Dosages of the adjuvants can vary according to the specific adjuvants. For example, in some aspects, dosage ranges can include: 10 μg/dose to 500 μg/dose, or 50 μg/dose to 200 μg/dose for CpG. Dosage ranges can include: 2 μg/dose to 100 μg/dose, or 10 μg/dose to 30 μg/dose for MPL. Dosage ranges can include: 10 μg/close to 500 μg/dose, or 50 mg/dose to 100 μg/dose for aluminum hydroxide. In a prime-boost regimen, as described elsewhere herein, an adjuvant may be used with either the priming immunization, the booster immunization, or both.

In certain adjuvant compositions, the adjuvant is a cytokine. Certain compositions of the present invention comprise one or more cytokines, chemokines, or compounds that induce the production of cytokines and chemokines, or a polynucleotide encoding one or more cytokines, chemokines, or compounds that induce the production of cytokines and chemokines. Examples of cytokines include, but are not limited to granulocyte macrophage colony stimulating factor (GM-CSF), granulocyte colony stimulating factor (G-CSF), macrophage colony stimulating factor (M-CSF), colony stimulating factor (CSF), erythropoietin (EPO), interleukin 2 (IL-2), interleukin-3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-10), interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interferon alpha (IFN), interferon beta (IFN), interferon gamma (IFN), interferon omega (IFN), interferon tau (IFN), interferon gamma inducing factor I (IGIF), transforming growth factor beta (TGF-), RANTES (regulated upon activation, normal T-cell expressed and presumably secreted), macrophage inflammatory proteins (e.g., MIP-1 alpha and MIP-1 beta), Leishmania elongation initiating factor (LEIF), and Flt-3 ligand.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th₂ response into a primarily cellular, or Th₁ response. Immune responses to a given antigen may be tested by various immunoassays well known to those of ordinary skill in the art, and/or described elsewhere herein.

Furthermore, the HtrA antigen may be conjugated to a bacterial toxoid, such as a toxoid from diphtheria, tetanus, cholera, H. pylori, or other pathogen. Furthermore, the HtrA antigen may be conjugated to a bacterial polysaccharide, such as the capsular polysaccharide from Neisseria sp., Streptococcus pneumoniae sp. or Haemophilus influenzae type-b bacteria.

In one embodiment of the invention, a polypeptide of the invention is expressed by an attenuated bacterial (e.g., Salmonella sp.) or viral vaccine carrier (e.g., MVA). The present invention thus includes compositions comprising an attenuated bacterial or viral vaccine carrier which expresses a Chlamydia HtrA polypeptide. The nucleic acid encoding the HtrA polypeptide may be in a vector or may be incorporated into the genome of the host carrier (for instance, by homologous recombination). In one embodiment of the invention, the vaccine composition comprises an attenuated Salmonella vaccine carrier comprising an attenuating mutation in a Salmonella Pathogenicity Island 2 region and, optionally, one or more additional attenuating mutations (for instance, a mutation in one or more of the aroA, aroC or sod genes) and a nucleic acid encoding a Chlamydia HtrA polypeptide. In another embodiment, the pharmaceutical composition comprises MVA comprising a nucleic acid encoding a Chlamydia HtrA polypeptide. The attenuated live vaccine compositions of the present invention can further comprise, for instance, a pharmaceutically acceptable carrier, diluent and/or adjuvant. See for instance, U.S. Pat. Nos. 6,342,215; 6,936,425; 6,756,042; and 7,211,264 which are herein incorporated by reference in their entireties.

The polypeptides of the invention can also be administered via liposome carriers, which serve to target the polypeptides to a particular tissue, such as lymphoid tissue, or to target selectively to infected cells, as well as to increase the half-life of the polypeptide composition. Liposomes include emulsions, foams, micelles, insoluble monolayers, liquid crystals, phospholipid dispersions, lamellar layers and the like. In these preparations, the polypeptide to be delivered is incorporated as part of a liposome, alone or in conjunction with a molecule which binds to a receptor prevalent among lymphoid cells (such as monoclonal antibodies which bind to the CD45 antigen or other costimulatory factor) or with other therapeutic or immunogenic compositions. Thus, liposomes either filled or decorated with a desired polypeptide of the invention can be directed to the site of lymphoid cells, where the liposomes then deliver the polypeptide compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), and U.S. Pat. Nos. 4,235,871, 4,501,728, 4,837,028, and 5,019,369. A liposome suspension containing a polypeptide of the invention may be administered intravenously, locally, topically, etc. in a dose which varies according to, inter alia, the manner of administration, the polypeptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more polypeptides of the invention, often at a concentration of 25%-75%.

For aerosol or mucosal administration, the immunogenic polypeptides can be supplied in finely divided form, optionally along with a surfactant and, propellant and/or a mucoadhesive, e.g., chitosan. Typical percentages of polypeptides are 0.01%-20% by weight, often 1%-10%. The surfactant must, of course, be pharmaceutically acceptable, and in some embodiments soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, in some embodiments 0.25-5% by weight. The balance of the composition is ordinarily propellant, although an atomizer may be used in which no propellant is necessary and other percentages are adjusted accordingly. In some embodiments, the immunogenic polypeptides can be incorporated within an aerodynamically light particle, such as those particles described in U.S. Pat. No. 6,942,868 or U.S. Pat. Pub. No. 2005/0008633. A carrier can also be included, e.g., lecithin for intranasal delivery.

In some embodiments, the present invention is directed to a multivalent vaccine. For example, a multivalent vaccine of the present invention can comprise a polypeptide at least 70%, 75%, 80%, 85%, 90%, 95%, 96%, 97%, 98%, or 99% identical to C. trachomatis HtrA sequences, fragments, derivatives, analogs, or variants described herein, e.g., in Tables 3-8 or any combinations of sequences thereof, wherein the polypeptide when administered to a subject in need thereof in a sufficient amount, induces an immune response against Chlamydia sp. and a polypeptide that elicits an immune reaction to one or more additional organisms and/or viruses, e.g., Haemophilus influenzae type b, Hepatitis B virus, Hepatitis A virus, Hepatitis C virus, Corynebacterium diphtheriae, Clostridium tetani, Polio virus, Influenza virus, Rubeola virus, Rubella virus, myxovirus, Neisseria, e.g., N. gonnorrheae, Haemophilus ducrey, Granuloma inguinale, Calymmatobacterium granulomatis, human papilloma virus (HPV) type I and II, Ureaplasma urealyticum, Mycoplasma hominis, Treponema pallidum, Poxvirus of the Molluscipox virus genus, Human Immunodeficiency Virus (HIV), Epstein-Barr virus (EBV), herpes simplex virus, varicella-zoster virus, or other Chlamydia species.

In some embodiments, the multivalent vaccine of the present invention can comprise a polypeptide of the present invention and a compatible vaccine, wherein both the vaccine of the present invention and the compatible vaccine are targeted for a similar patient population, e.g., an adolescent population. Examples of multivalent vaccines targeted for a specific patient population include, but are not limited to a vaccine for administration to an adolescent comprising a polypeptide of the present invention and a polypeptide that elicits an immune response to one or more of Hepatitis B virus, Hepatitis C virus, Neisseria, e.g., N. gonorrheae, Epstein-Barr virus (EBV), varicella-zoster virus, herpes simplex virus, human papilloma virus, or other Chlamydia species.

### Polynucleotide Vaccines

In some embodiments, the present invention is also directed to a polynucleotide vaccine. The term “DNA vaccine” or “polynucleotide vaccine” refers to a composition comprising the polypeptides of the present invention which when administered to an animal, e.g., in a viral vector, express one or more polypeptides of the present invention in the cells of the animal, thereby stimulating an immune response against Chlamydia infection.

Such polynucleotide vaccine compositions can include those adjuvants, carriers, excipients, or modes of administration listed herein for polypeptide vaccines. In some embodiments, if the adjuvant, carrier, or excipient is a polypeptide, the polynucleotide vaccine composition can comprise a nucleic acid fragment which encodes the adjuvant, carrier or excipient polypeptide. Polynucleotide vaccine compositions can also include, for example, naked DNA, DNA formulated with PVP, DNA in cationic lipid formulations; DNA encapsulated e.g., in poly(DL-lactide-co-glycolide) (“PLG”) microspheres (see, e.g., Eldridge, et al., Molec. Immunol. 28:287-294, 1991: Alonso et al., Vaccine 12:299-306, 1994; Jones et al., Vaccine 13:675-681, 1995), viral, bacterial, or, fungal delivery vectors (Perkus, M. E. et al., In: Concepts in vaccine development, Kaufmann, S. H. E., ed., p. 379, 1996; Chakrabarti, S. et al., Nature 320:535, 1986; Hu, S. L. et al., Nature 320:537, 1986; Kieny, M.-P. et al., AIDS Bio/Technology 4:790, 1986; Top, F. H. et al., J. Infect. Dis. 124:148, 1971; Chanda, P. K. et al., Virology 175:535, 1990); or particle-absorbed DNA (Ulmer, J. B. et al., Science 259:1745, 1993; Robinson, H. L., Hunt, L. A., and Webster, R. G., Vaccine 11:957, 1993; Shiver, J. W. et al., In: Concepts in vaccine development, Kaufmann, S. H. E., ed., p. 423, 1996; Cease, K. B., and Berzofsky, J. A., Annu. Rev. Immunol. 12:923, 1994 and Eldridge, J. H. et al., Sem. Hematol. 30:16, 1993), etc.

The present invention provides compositions comprising a polynucleotide encoding a polypeptide at least 70%, 75%, 80%, 85%, 90%, 95%, 96%, 97%, 98%, 99%, or 100% identical to C. trachomatis HtrA sequences, fragments, derivatives, analogs, or variants described herein. For example, the present invention includes a polynucleotide comprising a nucleic acid encoding an amino acid sequence described in Tables 3, 4, 5, 6, 7, or 8. A polynucleotide-based vaccine, or polynucleotide vaccine, of the present invention is capable of eliciting, without more, an immune response in an animal against a Chlamydia species, e.g., C. trachomatis, when administered to that animal.

Polynucleotide-based vaccines compositions of the invention include nucleic acid-mediated modalities. DNA or RNA encoding one or more of the polypeptides of the invention can also be administered to a patient. This approach is described, for instance, in Wolff et. al., Science 247: 1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA,” facilitated (bupivicaine, polymers, peptide-mediated) delivery, cationic lipid complexes, and particle-mediated (“gene gun”) or pressure-mediated delivery (see, e.g., U.S. Pat. No. 5,922,687).

In some embodiments, the polynucleotide-based vaccines are prepared and administered in such a manner that the encoded gene products are optimally expressed in the particular animal to which the composition is administered. As a result, these compositions and methods are useful in stimulating an immune response against Chlamydia infection as the coding sequence encodes a polypeptide which stimulates the immune system to respond to Chlamydia infection. Also included in the invention are expression systems, delivery systems, and codon-optimized Chlamydia coding sequences, e.g., viral vectors. Vaccinia vectors and methods useful in immunization protocols are described in, e.g., U.S. Pat. No. 4,722,848. Another vector is BCG (Bacille Calmette Guerin). BCG vectors are described in Stover et al., Nature 351:456-460 (1991). A wide variety of other vectors useful for therapeutic administration or immunization of the polypeptides of the invention, e.g. adeno and adeno-associated virus vectors, retroviral vectors, Salmonella typhi vectors, detoxified anthrax toxin vectors, and the like, will be apparent to those skilled in the art from the description herein.

In certain embodiments, the polynucleotides are complexed in a liposome preparation (Feigner et al., Proc. Natl. Acad. Sci. USA 84:74137416 (1987); Malone et al., Proc. Natl. Acad. Sci. USA 86:60776081 (1989)). Furthermore, polynucleotide-vaccine compositions of the present invention may include one or more transfection facilitating compounds that facilitate delivery of polynucleotides to the interior of a cell, and/or to a desired location within a cell.

In other embodiments, the polynucleotide itself may function as an adjuvant as is the case when the polynucleotides of the invention are derived, in whole or in part, from bacterial DNA. Bacterial DNA containing motifs of unmethylated CpG-dinucleotides (CpG-DNA) triggers innate immune cells in animals through a pattern recognition receptor (including toll receptors such as TLR 9) and thus possesses potent immunostimulatory effects on macrophages, dendritic cells and B-lymphocytes. See, e.g., Wagner, H., Curr. Opin. Microbiol. 5:62-69 (2002); Jung, J. et al., J. Immunol. 169: 2368-73 (2002); see also Klinman, D. M. et al., Proc. Natl. Acad. Sci. U.S.A. 93:2879-83 (1996). Methods of using unmethylated CpG-dinucleotides as adjuvants are described in, for example, U.S. Pat. Nos. 6,207,646, 6,406,705, and 6,429,199, the disclosures of which are herein incorporated by reference.

Compositions comprising polynucleotides of the present invention may include various salts, excipients, delivery vehicles and/or auxiliary agents as are disclosed, e.g., in U.S. Pat. No. 6,875,748, which is incorporated herein by reference in its entirety.

Embodiments of the vaccine compositions of the present invention also include, but are not limited to, a polypeptide encoded by a polynucleotide selected from the group consisting of:
(a) SEQ ID NO: 1;
(b) a sequence that hybridizes to SEQ ID NO: 1 or its complement under conditions of moderate stringency;
(c) a sequence that hybridizes to SEQ ID NO: 1 or its complement under conditions of high stringency; and
(d) a sequence that hybridizes to SEQ ID NO: 1 or its complement under conditions of very high stringency,
   provided that the polypeptide is not SEQ ID NO: 6 or SEQ ID NO: 8.

Direct delivery of the compositions will generally be accomplished by injection, either subcutaneously, intraperitoneally, intravenously, intranasally, intramuscularly, intravaginal, or intrarectal delivered to the interstitial space of a tissue, or administered to a mucosal surface, for example, intranasally. The compositions can also be administered into a lesion. Other modes of administration include oral and pulmonary administration, suppositories, and transdermal or transcutaneous applications. Dosage treatment may be a single dose schedule or a multiple dose schedule.

### Methods of Treatment/Prevention and Regimens

Also provided is a method to treat or prevent a Chlamydia infection in a subject comprising: administering to the animal in need thereof a composition containing any one or more of the polypeptides, polynucleotides, vectors, or host cells of the present invention. In certain embodiments, the subject is a vertebrate, e.g., a mammal, e.g., a primate, e.g., a human. In some embodiments, the invention is directed to a method of inducing an immune response against Chlamydia in a subject, e.g., a host animal comprising administering an effective amount a composition containing any one or more of the polypeptides, polynucleotides, vectors, or host cells of the present invention.

In some embodiments, an animal can be treated with the compositions, polypeptides, polynucleotides, vectors, or host cells prophylactically, e.g., as a prophylactic vaccine, to establish or enhance immunity to one or more Chlamydia species, e.g., Chlamydia trachomatis or Chlamydia pneumoniae, in a healthy animal prior to exposure to Chlamydia or contraction of a Chlamydia symptom, thus preventing the disease or reducing the severity of disease symptoms. One or more compositions, polypeptides, polynucleotides, vectors, or host cells of the present invention may also be used to treat an animal already exposed to Chlamydia, or already suffering from Chlamydia-related symptom to further stimulate the immune system of the animal, thus reducing or eliminating the symptoms associated with that exposure. As defined herein, “treatment of an animal” refers to the use of one or more compositions, polypeptides, polynucleotides, vectors, or host cells of the present invention to prevent, cure, retard, or reduce the severity of Chlamydia symptoms in an animal, and/or result in no worsening of Chlamydia symptoms over a specified period of time. It is not required that any composition, polypeptide, polynucleotide, a vector, or a host cell of the present invention provides total protection against Chlamydia infection or totally cure or eliminate all Chlamydia symptoms. As used herein, “an animal in need of therapeutic and/or preventative immunity” refers to an animal which it is desirable to treat, i.e., to prevent, cure, retard, or reduce the severity of Chlamydia symptoms, and/or result in no worsening of Chlamydia symptoms over a specified period of time.

In some embodiments, an antibody specifically reactive with a Chlamydia organism is isolated from the serum of the host animal which has been administered a polypeptide or polynucleotide of the present invention. In some embodiments, the invention is directed to a method of providing passive immunity comprising administering the antibody specifically reactive with a Chlamydia organism (which was isolated from the serum of a host animal) to an animal in need thereof.

Treatment with pharmaceutical compositions comprising the immunogenic compositions, polypeptides or polynucleotides of the present inventions can occur separately or in conjunction with other treatments, as appropriate.

In therapeutic applications, compositions, polypeptides or polynucleotides are administered to a patient in an amount sufficient to elicit an effective CTL response to the Chlamydia-derived polypeptide to cure or at least partially arrest symptoms and/or complications. An amount adequate to accomplish this is defined as “therapeutically effective dose” or “unit dose.” Amounts effective for this use will depend on, e.g., the polypeptide or polynucleotide composition, the manner of administration, the stage and severity of the disease being treated, the weight and general state of health of the patient, and the judgment of the prescribing physician, but generally range for the initial immunization for polypeptide vaccines is (that is for therapeutic or prophylactic administration) from about 1.0 μg to about 5000 μg of polypeptide, in some embodiments about 10 μg to about 30 μg, for a 70 kg patient, followed by boosting dosages of from about 1.0 μg to about 1000 μg, in some embodiments 10 μg to about 30 μg, of polypeptide pursuant to a boosting regimen over weeks to months depending upon the patient's response and condition by measuring specific CTL activity in the patient's blood. In alternative embodiments, generally for humans the dose range for the initial immunization (that is for therapeutic or prophylactic administration) is from about 1.0 μg to about 20,000 μg of polypeptide for a 70 kg patient, in some embodiments 2 μg-, 5-10 μg-, 15 μg-, 20 μg-, 25 μg-, 30 μg-, 40 μg-, or 50 μg-2000 μg, followed by boosting dosages in the same dose range pursuant to a boosting regimen over weeks to months depending upon the patient's response and condition by measuring specific CTL (cytotoxic T lymphocytes) activity in the patient's blood. In a specific, non-limiting embodiment of the invention, approximately 0.01 to 2000 μg, or in some embodiments 2 μg to 200 μg or 10 μg to 30 μg, of a polypeptide or polynucleotide of the present invention, or its fragment, derivative variant, or analog is administered to a host.

In embodiments where DNA vaccine administration is used, the amount of polynucleotide in the initial immunization (that is for therapeutic or prophylactic administration) depends upon a number of factors including, for example, the antigen being expressed, the expression vector being used, the age and weight of the subject, the precise condition requiring treatment and its severity, and the route of administration. Based on the above factors, determining the precise amount, number of doses, and timing of doses are within the ordinary skill in the art and will be readily determined by the attending physician or veterinarian. In some embodiments, doses for nucleic acids encoding polypeptides range from about 10 ng to 1 g, 100 ng to 100 mg, 1 μg to 10 mg, or 30-300 μg DNA or RNA per patient.

In non-limiting embodiments of the invention, an effective amount of a composition of the invention produces an elevation of antibody titer to at least three times the antibody titer prior to administration.

It must be kept in mind that the polypeptides and compositions of the present invention may generally be employed in serious disease states, that is, life-threatening or potentially life threatening situations. In such cases, in view of the minimization of extraneous substances and the relative nontoxic nature of the polypeptides, it is possible and may be felt desirable by the treating physician to administer substantial excesses of these polypeptide compositions.

For therapeutic use, administration should begin at the first sign of Chlamydia infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

More specifically, the compositions of the present invention may be administered to any tissue of an animal, including, but not limited to, muscle, skin, brain tissue, lung tissue, liver tissue, spleen tissue, bone marrow tissue, thymus tissue, heart tissue, e.g., myocardium, endocardium, and pericardium, lymph tissue, blood tissue, bone tissue, pancreas tissue, kidney tissue, gall bladder tissue, stomach tissue, intestinal tissue, testicular tissue, ovarian tissue, uterine tissue, vaginal tissue, rectal tissue, nervous system tissue, eye tissue, glandular tissue, tongue tissue, and connective tissue, e.g., cartilage.

Furthermore, the compositions of the present invention may be administered to any internal cavity of a vertebrate, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, the ocular cavities, the lumen of a duct of a salivary gland or a liver. When the compositions of the present invention is administered to the lumen of a duct of a salivary gland or a liver, the desired polypeptide is encoded in each of the salivary gland and the liver such that the polypeptide is delivered into the blood stream of the vertebrate from each of the salivary gland and the liver. Certain modes for administration to secretory organs of a gastrointestinal system using the salivary gland, liver and pancreas to release a desired polypeptide into the bloodstream is disclosed in U.S. Pat. Nos. 5,837,693 and 6,004,944, both of which are incorporated herein by reference in their entireties.

In one embodiment, the compositions are administered to muscle, either skeletal muscle or cardiac muscle, or lung tissue. Specific, but non-limiting modes for administration to lung tissue are disclosed in Wheeler, C. J., et al., Proc. Natl. Acad. Sci. USA 93:11454-11459 (1996), which is incorporated herein by reference in its entirety.

In certain embodiments, one or more compositions of the present invention are delivered to an animal by methods described herein, thereby achieving an effective immune response, and/or an effective therapeutic or preventative immune response. Any mode of administration can be used so long as the mode results in the delivery and/or expression of the desired polypeptide in the desired tissue, in an amount sufficient to generate an immune response to Chlamydia, e.g., C. trachomatis, and/or to generate a prophylactically or therapeutically effective immune response to Chlamydia, e.g., C. trachomatis, in an animal in need of such response. According to the disclosed methods, compositions of the present invention can be administered by mucosal delivery, transdermal delivery, subcutaneous injection, intravenous injection, oral administration, pulmonary administration, intramuscular (i.m.) administration, or via intradural injection. Other suitable routes of administration include, but not limited to intratracheal, transdermal, intraocular, intranasal, inhalation, intracavity, intraductal (e.g., into the pancreas) and intraparenchymal (i.e., into any tissue) administration. Transdermal delivery includes, but not limited to intradermal (e.g., into the dermis or epidermis), transdermal (e.g., percutaneous) and transmucosal administration (i.e., into or through skin or mucosal tissue). Intracavity administration includes, but not limited to administration into oral, vaginal, rectal, nasal, peritoneal, or intestinal cavities as well as, intrathecal (i.e., into spinal canal), intraventricular (i.e., into the brain ventricles or the heart ventricles), intraatrial (i.e., into the heart atrium) and sub arachnoid (i.e., into the sub arachnoid spaces of the brain) administration.

Any mode of administration can be used so long as the mode results in the delivery and/or expression of the desired polypeptide in the desired tissue, in an amount sufficient to generate an immune response to Chlamydia, e.g., C. trachomatis, and/or to generate a prophylactically or therapeutically effective immune response to Chlamydia, e.g., C. trachomatis, in an animal in need of such response. Administration means of the present invention include needle injection, catheter infusion, biolistic injectors, particle accelerators (e.g., “gene guns” or pneumatic “needleless” injectors) Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet (Linmayer, I., et al., Diabetes Care 9:294-297 (1986)), Medi-jector (Martins, J., and Roedl, E. J. Occup. Med. 21:821-824 (1979)), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, Y., et al., Life Sciences 65, 2193-2203 (1999)) or topical applications during surgery. Certain modes of administration are intramuscular needle-based injection and pulmonary application via catheter infusion. Each of the references cited in this paragraph is incorporated herein by reference in its entirety.

Upon immunization with a polypeptide or polynucleotide composition in accordance with the invention, the immune system of the host responds to the vaccine by producing large amounts of HTLs (helper T lymphocytes) and/or CTLs (cytotoxic T lymphocytes) specific for the desired antigen. Consequently, the host becomes at least partially immune to later infection, or at least partially resistant to developing an ongoing chronic infection.

In some embodiments, polypeptides or polynucleotides of the present invention stimulate a cell-mediated immune response sufficient for protection of an animal against Chlamydia infection. In other embodiments, polypeptides or polynucleotides of the present invention stimulate both a humoral and a cell-mediated response, the combination of which is sufficient for protection of an animal against Chlamydia protection.

In certain embodiments, components that induce T cell responses are combined with components that induce antibody responses to the target antigen of interest. Thus, in certain embodiments of the invention, vaccine compositions of the invention are combined with polypeptides or polynucleotides which induce or facilitate neutralizing antibody responses to the target antigen of interest. One embodiment of such a composition comprises a class I epitope in accordance with the invention, along with a PADRE® (Epimmune, San Diego, Calif.) molecule (described, for example, in U.S. Pat. No. 5,736,142, which is incorporated herein by reference in its entirety.).

The polynucleotides of the present invention, or vectors containing the polynucleotides of the present invention, can be incorporated into the cells of the animal in vivo, and an antigenic amount of the C. trachomatis-derived polypeptide, or fragments, variants, or derivatives thereof, is produced in vivo. Upon administration of the composition according to this method, the C. trachomatis-derived polypeptide is expressed in the animal in an amount sufficient to elicit an immune response. Such an immune response might be used, for example, to generate antibodies to C. trachomatis for use in diagnostic assays or as laboratory reagents.

The present invention further provides a method for generating, enhancing, or modulating a protective and/or therapeutic immune response to C. trachomatis in an animal, comprising administering to the animal in need of therapeutic and/or preventative immunity one or more of the compositions described herein. In some embodiments, the composition includes an isolated polynucleotide comprising a codon-optimized coding region encoding a polypeptide of the present invention, optimized for expression in a given host organism, e.g., a human, or a nucleic acid fragment of such a coding region encoding a fragment, variant, or derivative thereof. The polynucleotides are incorporated into the cells of the animal in vivo, and an immunologically effective amount of the C. trachomatis polypeptide, or fragment or variant is produced in vivo. Upon administration of the composition according to this method, the C. trachomatis-derived polypeptide is expressed in the animal in a therapeutically or prophylactically effective amount.

The compositions of the present invention can be administered to an animal at any time during the lifecycle of the animal to which it is being administered. For example, the composition can be given shortly after birth. In humans, administration of the composition of the present invention can occur while other vaccines are being administered, e.g., at birth, 2 months, 4 months, 6 months, 9 months, at 1 year, at 5 years, or at the onset of puberty. In some embodiments, administration of the composition of the present invention can occur when the human become sexually active.

Furthermore, the compositions of the invention can be used in any desired immunization or administration regimen; e.g., in a single administration or alternatively as part of periodic vaccinations such as annual vaccinations, or as in a prime-boost regime wherein the polypeptide or polynucleotide of the present invention is administered either before or after the administration of the same or of a different polypeptide or polynucleotide.

Recent studies have indicated that a prime-boost protocol is often a suitable method of administering vaccines. In a prime-boost protocol, one or more compositions of the present invention can be utilized in a “prime boost” regimen. An example of a “prime boost” regimen may be found in Yang, Z. et al. J. Virol. 77:799-803 (2002), which is incorporated herein by reference in its entirety. In a non-limiting example, one or more polynucleotide vaccine compositions of the present invention are delivered to an animal, thereby priming the immune response of the animal to a Chlamydia polypeptide of the invention, and then a second immunogenic composition is utilized as a boost vaccination. One or more compositions of the present invention are used to prime immunity, and then a second immunogenic composition, e.g., a recombinant viral vaccine or vaccines, e.g., a recombinant vaccine virus, e.g., a recombinant MVA vaccinia virus, a different polynucleotide vaccine, or one or more purified subunit of the Chlamydia polypeptides or fragments, variants or derivatives thereof is used to boost the anti-Chlamydia immune response.

In another non-limiting example, a priming composition and a boosting composition are combined in a single composition or single formulation. For example, a single composition may comprise an isolated Chlamydia polypeptide or a fragment, variant, or derivative thereof as the priming component and a polynucleotide encoding a Chlamydia polypeptide as the boosting component. In this embodiment, the compositions may be contained in a single vial where the priming component and boosting component are mixed together. In general, because the peak levels of expression of polypeptide from the polynucleotide does not occur until later (e.g., 7-10 days) after administration, the polynucleotide component may provide a boost to the isolated polypeptide component. Compositions comprising both a priming component and a boosting component are referred to herein as “combinatorial vaccine compositions” or “single formulation heterologous prime-boost vaccine compositions.” In addition, the priming composition may be administered before the boosting composition, or even after the boosting composition, if the boosting composition is expected to take longer to act.

In another embodiment, the priming composition may be administered simultaneously with the boosting composition, but in separate formulations where the priming component and the boosting component are separated.

### Kits

The polypeptide or polynucleotide vaccine compositions of this invention can be provided in kit form together with a means for administering the polypeptide, polynucleotide, or composition of the present invention. In some embodiments, the kit can further comprise instructions for vaccine administration.

Typically the kit would include desired composition(s) of the invention in a container, e.g., in unit dosage form and instructions for administration. Means for administering the composition of the present invention can include, for example, a sterile syringe, an aerosol applicator (e.g., an inhaler or any other means of nasal or pulmonary administration), a gel, a cream, a transdermal patch, transmucosal patch (or any other means of buccal or sublingual administration), or an oral tablet. In some embodiments, the kit of the present invention contains two or more means for administering the polypeptides, polynucleotides, vectors, or compositions of the present inventions, e.g., two or more syringes.

In some embodiments, the kit may comprise more than one container comprising the polypeptide, polynucleotide, or composition of the present invention. For example, in some embodiments the kit may comprise a container containing a priming component of the present invention, and a separate container comprising the boosting component of the present invention.

Optionally associated with such container(s) can be a notice or printed instructions. For example, such printed instructions can be in a form prescribed by a governmental agency regulating the manufacture, use or sale of pharmaceuticals or biological products, which notice reflects approval by the agency of the manufacture, use or sale for human administration of the present invention. “Printed instructions” can be, for example, one of a book, booklet, brochure or leaflet.

The kit can also include a storage unit for storing the components (e.g., means of administering, containers comprising the polypeptides, polynucleotides, or compositions of the present inventions, printed instructions, etc.) of the kit. The storage unit can be, for example, a bag, box, envelope or any other container that would be suitable for use in the present invention. Preferably, the storage unit is large enough to accommodate each component that may be necessary for administering the methods of the present invention.

The present invention can also include a method of delivering a polypeptide, polynucleotide, or composition of the present invention to an animal such as a human in need thereof, the method comprising (a) registering in a computer readable medium the identity of an administrator (e.g., a physician, physician assistant, nurse practitioner, pharmacist, veterinarian) permitted to administer the polypeptide, polynucleotide, vector, or composition of the present invention; (b) providing the human with counseling information concerning the risks attendant the polypeptide, polynucleotide, vector, or composition of the present invention; (c) obtaining informed consent from the human to receive the polypeptide, polynucleotide, vector, or composition of the present invention despite the attendant risks; and (e) permitting the human access to the polypeptide, polynucleotide, vector, or composition of the present invention.

### Immunoassays

The present invention includes a method of detecting Chlamydia in a test sample. In certain embodiments, the presence of Chlamydia species can be detected by contacting the test sample with the antibody against C. trachomatis HtrA polypeptides, fragments, derivatives, analogs, or variants thereof to form antigen:antibody immunocomplexes and further detecting the presence of or measuring the amount of said immunocomplexes formed during the step. The present invention also provides assays for detecting or measuring an immune response to C. trachomatis HtrA polypeptides of the present invention. In some embodiments, the immune response of an organism can be determined by comparing the sera from an organism that is unvaccinated, or that has not been exposed to an antigen originating from Chlamydia (preimmune sera), to the sera from an organism that has been vaccinated, or that has been exposed to an antigen originating from Chlamydia (immune sera). As used herein, “a detectable immune response” refers to an immunogenic response to the polynucleotides and polypeptides of the present invention, which can be measured or observed by standard protocols. For example, the immune response can be either antibody response or T-cell response. Thus, the present invention provides a method of detecting antibodies against Chlamydia in a test sample comprising the steps of contacting the test sample with C. trachomatis HtrA polypeptides to form antigen: antibody immunocomplexes, and further detecting the presence of or measuring the amount of the immunocomplexes formed during the step. The present invention also provides a method for determining the presence of nucleic acids of Chlamydia sp. in a test sample, comprising the steps of contacting the test sample with the polynucleotide described herein or complement sequence thereof to produce duplexes and determining the production of duplexes. The test sample can be a patient specimen, e.g., tissues, blood, serum, saliva, mucous, and any organs or the patient itself.

Standard protocols for detecting an immune response include, but are not limited to, immunoblot analysis (western), fluorescence-activated cell sorting (FACS), radioimmunoassays, ELISA (enzyme linked immunosorbent assay), “sandwich” immunoassays, immunoprecipitation analysis, cytolytic T-cell response, ELISPOT, and chromium release assay. An immune response may also be “detected” through challenge of immunized animals with a virulent Chlamydia species, either before or after vaccination. Standard chromium release assays are used to measure specific cytotoxic T lymphocyte (CTL) activity against the Chlamydia antigens. More sensitive techniques such as the ELISPOT assay, intracellular cytokine staining, and tetramer staining have become available in the art to determine lymphocyte antigen responsiveness. It is estimated that these newer methods are 10- to 100-fold more sensitive than the common CTL and HTL assays (Murali-Krishna et al., Immunity, 8:177-87 (1998)), because the traditional methods measure only the subset of T cells that can proliferate in vitro, and may, in fact, be representative of only a fraction of the memory T cell compartment (Ogg G. S., McMichael A. J., Curr Opin Immunol, 10:393-6 (1998)).

Western blot analysis generally comprises preparing protein samples, e.g., polypeptides of the present invention, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-TWEEN 20™), blocking the membrane with primary antibody, e.g., serum from vaccinated individuals, preimmune sera and control positive antibodies, diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. A person of ordinary skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York. Vol. 1 (1994) at 10.8.1.

ELISAs comprise preparing polypeptide of the invention, coating the well of a 96 well microtiter plate with a polypeptide of the invention, adding test antibodies (e.g., from immune sera in serial dilutions) and control antibodies (e.g., from preimmune sera) to the microtiter plate as described above, and incubating for a period of time. Then a second antibody (which recognizes the antibody of interest) conjugated to a detectable compound may be added to the well, wherein the second antibody is conjugated to a detectable compound such as an enzymatic substrate. A person of ordinary skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunology 4^{th} ed. Ed. Richard A. Goldsby, Thomas J. Kindt and Barbara A. Osborne, H. Freemand & Co. (2000); Roitt, I., Brostoff, J. and Male D, Immunology, 6^{th} ed. London: Mosby (2001); Abbas A., Abul, A. and Lichtman, A., Cellular and Molecular Immunology, Ed. 5, Elsevier Health Sciences Division (2005); Kontermann and Dubel, Antibody Engineering, Springer Verlan (2001); Sambrook and Russell, Molecular Cloning: A Laboratory Manual, Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer, Cold Spring Harbor Press (2003).

### Biological Deposits

Biological deposits will be made with the American Type Culture Collection (ATCC) located at 10801 University Boulevard, Manassas, Va. 20110-2209, U.S.A., pursuant to the Budapest Treaty and pursuant to 37 C.F.R. §1.808. Samples of the deposited materials will become available to the public upon grant of a patent. The invention described and claimed herein is not to be limited by the scope of the strain deposited, since the deposited embodiment is intended only as an illustration of the invention.

| | ATCC | |
|---|---|---|
| strain | Accession No. | Date Deposited |
| E. coli BL21(DE3)(pET28-CtHtrA/L2) | PTA-8868 | Jan. 10, 2008 |

### EXAMPLES

### Example 1

### Cloning and Expression of C. trachomatis HtrA

A full length gene encoding C. trachomatis HtrA was isolated by PCR amplification of genomic DNA from C. trachomatis strain L₂ Bu434. Primers used for PCR amplification included synthetic restriction sites, NcoI, shown underlined in the forward primer, SEQ ID NO: 9, and SalI, shown underlined in the reverse primer, SEQ ID NO: 10. The reverse primer further included an artificial sequence coding 6 histidine residues, to facilitate detection and purification of the recombinant protein.

SEQ ID No. 9:
CCCGG CCATGG TG AAA AGA TTA TGT GTG TTG
SEQ ID No. 10:
CCGGGGTCGAC CTA GTG GTG GTG GTG GTG CTC GTC TGA
TTT CAA GAC GAT GAA

The amplification product was 1500 bp. After purification using a PCR column, the amplification product, containing a C. trachomatis HtrA gene, and also expression vector pET-28a(+) were digested with NcoI and SalI restriction enzymes and purified using agarose gel. The digested cloned C. trachomatis HtrA gene and pET-28a(+) vector were then ligated using T4 ligase. After ligation, the recombinant expression construct was transformed into BL21 (DE3) E. coli cells for clone selection. Antibiotic-resistant clones were picked at random and screened for the presence of HtrA-encoding inserts in the proper orientation for expression by conventional restriction endonuclease digestion.

To confirm successful expression of recombinant HtrA, BL21 (DE3) cells with or without pET28 constructs were cultured in LB medium at 37° C. for 5 hours. The cell cultures were then induced with 1 mmol IPTG for an additional 5 hours. The cells were collected and IPTG-inducible expressed proteins and separated based on molecular size by SDS-PAGE using commercially available NuPAGE™ gels. The proteins were then transferred to cellulose membrane and subjected to Western blot analysis. FIG. 4(A) shows the Western Blot of C. trachomatis HtrA polypeptide purified from E. coli BL21 cells using anti-PentaHis monoclonal antibody. Aliquots from two lots of purified recombinant C. trachomatis HtrA from the E. coli strain BL21 (DE3) (pET28-CtHtrA/L2) were electrophoresed in a 4-12% NuPAGE™ (Novex) gel, electroblotted onto a PVDF membrane, and subjected to immunoblotting using rabbit anti-PentaHis monoclonal sera as the primary antibody (1/5,000 dilution) (Qiagen, Valencia, Calif.) and a goat anti-mouse alkaline phosphatase-labelled secondary antibody (1/1,000 dilution) (Qiagen, Valencia, Calif.).

The sequence of the pET28 construct was determined using an AB310 sequencer with the following primers:

| | |
|---|---|
| SEQ ID NO: 11: | HtrA 1, forward ATGATGAAAAGATTATTA |
| SEQ ID NO: 12: | HtrA 500, forward TAGATCCAAAAACAGA |
| SEQ ID NO: 13: | HtrA 1060, forward AAGTAGAGTCTTTGA |
| | GTG |
| SEQ ID NO: 14: | HtrA 200, reverse ATATACAACTCCAGGC |
| | GT |
| SEQ ID NO: 15: | HtrA 550, reverse AAATGGTAATTTCTCC |
| | GC |
| SEQ ID NO: 16: | HtrA 1090, reverse GGAAATGGCATTACG |
| | CAA |
| SEQ ID NO: 17 | HtrA 1490, reverse CTACTCGTCTGATTT |
| | CAA |

The sequence of the HtrA gene inserted in pET28 contained 1494 by and is shown without the added 6 histidine tag as SEQ ID NO: 1. The deduced amino acid sequence of the open reading frame is shown without the added 6 histidine tag as SEQ ID NO: 2. The nucleotide sequence of the HtrA chimeric containing 6 histidine tag is shown as SEQ ID NO: 3, and the corresponding deduced amino acid sequence as SEQ ID NO: 4.

To further characterize the expressed product, recombinant 6 histidine tagged C. trachomatis HtrA polypeptide was purified from E. coli culture, subject to SDS-PAGE analysis and assayed for viable protease activity. Cell pellet from a 250 ml culture was resuspended in 20 ml 20 mM Tris, pH 8.0, 8 M urea, and lysed by sonification. The lysate was clarified by centrifugation at 25,000×g, 30 min, 4° C. Supernatant containing recombinant HtrA polypeptides was loaded at 5.0 ml/min onto a 5 ml HITRAP™ chelate column equilibrated with 20 mM Tris, pH 8.0, 8 M urea, 10 mM imidazole, washed with 6 column volumes of equilibration buffer, then further washed with three column volumes of the same buffer containing 20 mM imidazole and, finally, eluted with a 200 mM imidazole step gradient. Elution fractions of approximately 2.5 ml were collected and applied to a NuPAGE™ gel.

FIG. 4(B) shows protease activity of recombinant C. trachomatis HtrA in a zymograph gels. An aliquot of purified recombinant C. trachomatis HtrA from the E. coli strain BL21 (DE3) (pET28-CtHtrA/L2) was electrophoresed in a 4-16% BlueCasein zymogram gel (Novex) and developed overnight at 37° C. according to the manufacturers instructions. Recombinant C. trachomatis HtrA degradation (i.e. proteolysis) of the blue casein substrate is visualized as a zone of clearing at ˜49 kDa.

### Example 2

### Purification of Recombinant C. trachomatis HtrA Polypeptides for Use in Vaccine Compositions

Recombinant C. trachomatis HtrA polypeptides were expressed in BL21 E. coli cells with expression vector pET28 as described in Example 1. Cell paste (3 g) was suspended in 60 ml 25 mM Tris, pH 8.0 and sonicated at 50% duty cycle for 2 min on ice. Lysate was centrifuged at 25,000×g for 20 min at 4° C. Supernatant containing recombinant C. trachomatis HtrA was filtered with a 0.2 μM filter and applied at 1.5 ml/min in 25 mM Tris, pH 8.0, to a Ni-chelating column equilibrated with three column volumes of 25 mM Tris, pH8.0, containing 200 mM imidazole and again with three column volumes of 25 mM Tris, pH 8.0. The column was washed with seven column volumes of 25 mM Tris, pH 8.0, containing 20 mM imidazole, and eluted with 25 mM Tris, pH 8.0, containing 200 mM imidazole. Elution fractions of 2.0 ml were collected. Recombinant C. trachomatis HtrA eluted in fractions #4 through #7.

Ni-chelate elution fractions #4 through #7 were pooled, an equal volume added of 25 mM Na-acetate, pH 5.2, and pH adjusted to 5.2 using 1M HCl. This material was further purified by cation exchange using an HI-TRAP™ 1 ml column. Sample was applied at 1 ml/min to a column equilibrated in 25 mM Tris, pH 8.0, containing 200 mM imidazole, washed with six column volumes 25 mM Na-acetate, pH 5.2, and eluted at 1 ml/min with a step gradient at 35% of 25 mM Na-acetate, pH 5.2, containing 1M NaCl for 10 ml followed by a linear gradient of 36-100% in 30 ml. Low molecular weight impurities elute at the step gradient, while recombinant C. trachomatis HtrA elutes in the linear gradient.

Residual host cell endotoxin was removed by centrifugation. A sufficient quantity of chilled stock of 10% Triton X-114 in water was added to HI-TRAP™ eluates to reach final concentration about 2.5% Triton. The sample was allowed to stand on ice for 30 min, agitated once every 10 min, then warmed to room temperature. The sample was then centrifuged at room temperature at 17,000×g for 15 min in 1.5 ml EPPENDORF™ tubes or at 20,000×g for 20 min in 50 ml polypropylene centrifuge tubes. Clear supernatant containing recombinant C. trachomatis HtrA was dialysed once for three hours and once overnight against 100× volume of water.

Total protein was determined using a micro BCA protein assay. Endotoxin content was measured using a commercial spectrophotometric LAL endotoxin assay (Cape Code Associates). After endotoxin removal, the recombinant C. trachomatis HtrA preparations are substantially purified, at lest about 95% pure by weight (Data not shown.

FIG. 4(C) shows Coomassie blue stained SDS-PAGE of purified recombinant C. trachomatis HtrA samples suitable for use in vaccine compositions. Lane 1 is MARK-12™ molecular weight standards. Lane 2 is 4.2 μg HtrA. Lane 3 is 5.0 μg HtrA.

For Western blots, aliquots of purified HtrA were rehydrated in sample buffer, 6% BME, loaded on NuPAGE™ gels and run 40 min at 200V. The NuPAGE™ gel was transferred to a PVDF membrane which was incubated 1 hour at room temperature in blocking buffer consisting of 1% BSA in 1×TBS prior to immunostaining. The membrane was incubated for 2 hours at room temperature with agitation in first antibody, K306, a polyclonal rabbit anti-serum raised against recombinant C. pneumoniae HtrA prepared previously, at 1:50,000 dilution in blocking buffer. The membrane was washed in three 5 min washes in 1×TBS. After washing, a second antibody was added, alkaline-phosphatase labeled goat-anti-rabbit serum, at 1:1,000 dilution in blocking buffer. This solution was agitated 1 hour at room temperature, then subject to the same wash procedure. After final wash, the membrane was incubated in alkaline-phosphatase substrate for 10-15 minutes.

FIG. 4(D) shows a Western blot using rabbit polyclonal serum raised against C. pneumoniae HtrA. Aliquots from two lots of purified recombinant C. trachomatis HtrA from the E. coli strain BL21 (DE3) (pET28-CtHtrA/L2) were electrophoresed in a 4-12% NuPAGE™ (Novex) gel, electroblotted onto a PVDF membrane and subjected to immunoblotting using rabbit anti-Chlamydia pneumoniae polyclonal sera (K306) as the primary antibody (1/50,000 dilution) and a goat anti-rabbit alkaline phosphate-labelled secondary antibody (1/1,000 dilution). Lane 1 is SEEBLUE™ molecular weight standards. Lane 2 is recombinant C. trachomatis 4.2 μg HtrA and Lane 3 is 5.0 μg of recombinant C. trachomatis HtrA.

### Example 3

### Cellular Immune Response to Recombinant C. trachomatis HtrA

C. trachomatis E serovar EBs for use in immunization and challenge experiments were prepared as stock solutions and stored at −80° C. Infected cell cultures grown in culture flasks were ruptured by sonication and a supernatant enriched in EBs obtained by centrifugation of cell lysate for 500×g for 15 minutes at 4° C. The EB enriched supernatant was further purified by renografin (diatrizoate meglumine and diatrizoate sodium) density gradient centrifugation. An aliquot (2 ml) of EB enriched supernatant was gently added to the top of an 11 ml renografin gradient (2.5 ml of 54%, 4 ml of 44% and 1.5 ml of 40%) and centrifuged at 4° C. 1 hr at 18,000×g in a SORVALL™ OTD65B rotor. The EB-enriched layer usually between 44-54% renografin was collected, washed with PBS, and pelleted by centrifugation at 4° C. for 30 min at 13,000×g in a SORVALL™ RC5C rotor. The EB stock solution consisted of this pellet resuspended in 1.5 ml of SPG buffer (220 mM sucrose, 4 mM potassium phosphate, 9 mM sodium phosphate, 5 mM L-glutamic acid, pH 7.5) by brief sonication. Inclusion forming units (IFU) were determined per volume of live EB stock solution, or per vaginal swab experimental sample, using an in vitro cell infectivity assay employing McCoy cells. Monolayers of McCoy cells were grown in 96-well plates overnight at 37° C. in Dulbecco's Modified Eagle's medium (DMEM)-10. Culture medium was discarded and monolayers pre-treated for 20 minutes with 200 μl of DEAE-dextran in Hanks' balanced salt solution. This solution was discarded and aliquots of about 50 μl diluted EB stock or vaginal swab samples in SPG buffer were added. Plates were agitated by tilting every 20 minutes and cultured for two hours at 37° C. EB or vaginal swab samples were then removed, and replaced with 200 μl DMEM-10 with 50 μg/ml gentamycin and 1 μg/ml cycloheximide. Plates were incubated 1 to 2 days at 37° C. Culture medium was discarded and plates gently washed once with PBS. An aliquot of 100 μl of 0.01% hydrogen peroxide in absolute methanol was then added. Killed cells were washed with PBS. An aliquot of 100 μl of primary antibody, mouse anti-C. trachomatis serum at 1:100 dilution, was added and cells incubated for 1 hour at room temperature or overnight at 4° C. Cells were again washed with PBS. An aliquot of 50 μl of secondary antibody, goat anti-mouse conjugated to horseradish peroxidase at 1:200 dilution, was added and cells incubated for 1 hour at room temperature or overnight at 4° C. Cells were again washed with PBS. An aliquot of 50 μl of substrate composed of PBS with 0.1% chloro-naphthol in methanol was added. The substrate was removed and cells again washed with PBS. Stained C. trachomatis inclusion bodies were counted in five fields using light microscopy. IFU were determined as an extrapolation from the average count across five fields.

Immunogenic activity of recombinant C. trachomatis HtrA was evaluated in female C3H/HeOuJ mice, aged 6 to 8 weeks. All mice used in the study were tagged and bled 5 days before immunization. Groups of 10 mice were immunized three times with either 10 μg recombinant HtrA, 50 μg recombinant HtrA, or either 10 μg or 50 μg recombinant HtrA and 5 μg of mLT (used as an adjuvant candidate). Groups of 10 control mice were immunized three times with either PBS or live elementary bodies from C. trachomatis serovar E as controls.

All immunizations were intranasal with a total volume of 20 μl/dose. Mice in each experimental and control group were immunized on day 0, 14 and 22 of the study. Mice were non-fatally bled on day 21 and day 26, and serum samples stored at −20° C. Mouse vaginal tracts were lavaged using 60 μl PBS and samples stored at −20° C. on two occasions.

In each group, 4 mice were sacrificed at day 38 for spleen cell proliferation assays. Spleens were removed and spleen cell cultures maintained separately for tissues from each experimental animal.

At day 14 after the last immunization, subsets of mice were sacrificed for immunological evaluation. Spleen cell suspensions were cultured for cell proliferation assays and cytokine assays. For proliferation assays, cells (1.25×10⁶ cells/ml) were cultured in 96-well plates for 5 days in the presence or absence of Chlamydia antigen recombinant HtrA protein or UV-inactivated inclusion bodies at 1, 3, 10μ/ml. Concanavalin A (5 μg/ml, Sigma-Aldrich, St. Louis, Mo.) was used as a positive control in the assay. Methyl-3H thymidine (Amersham Biosciences Co., Piscataway, N.J.) at 1 μCi/well was added to the cultures during the last 18 to 24 hours of the day culture period. Pulsed cells were harvested and counted by liquid scintillation counting. Cell proliferation was expressed as the stimulation index (SI; CPM of triplicate antigen-restimulated wells/CPM of triplicate background wells for each animal).

For cytokine assays, spleen cells from individual mice were cultured at 2×10⁶ cells/ml in the presence or absence of Chlamydia antigen recombinant HtrA protein or UV-inactivated inclusion bodies at 1, 3, 10 μg/ml for 4 days. Culture supernatants were harvested and assayed for cytokine IFN-γ and IL-5 were purchased from BD PharMingen (San Diego, Calif.).

One-way ANOVA test was used to determine the significance of differences between groups using Prism software program version 3.0 (GraphPad Software Inc., San Diego, Calif.).

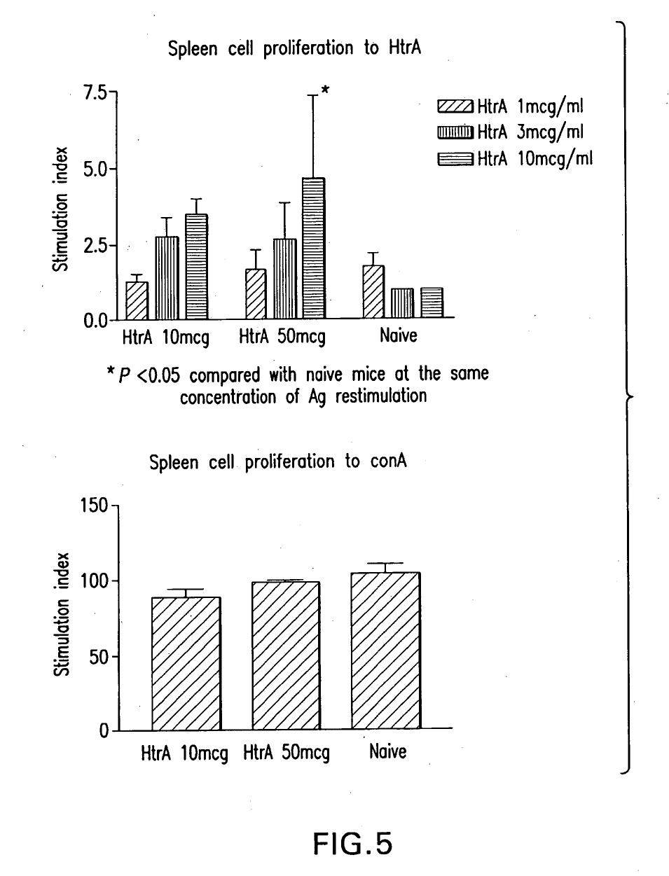

FIG. 5 shows spleen cell proliferative response to recombinant C. trachomatis HtrA. Spleens were removed and splenocyte cell cultures maintained separately for tissues from animals immunized with either 10 or 50 μg recombinant HtrA. Cultures were restimulated in vitro with either 1, 3, or 10 μg/ml of HtrA or ConA, as a negative control. Panel A shows stimulation index for each group of cultures restimulated with HtrA. Panel B shows stimulation index for each group of cultures challenged with ConA. * P<0.05 compared with naive mice at same concentration of antigen restimulation.

As shown in FIG. 5, spleen cell proliferation in response to restimulation with recombinant HtrA was enhanced relative to naive controls in tissues from animals previously immunized with either 10 or 50 μg HtrA. While the lowest restimulation concentration had no apparent effect, at both 3 μg/ml and 5 μg/ml HtrA, proliferation was enhanced relative to naive controls in tissues from animals immunized with either 10 or 50 μg HtrA. The effect was statistically significant at the highest restimulation dose of tissues from animals immunized with the highest dose of HtrA (P<0.05,). Spleen cell isolated from animals immunized with either 10 or 50 μg recombinant HtrA did not differ from those of naive animals in response to ConA, which was used as a negative control.

### Example 4

### Humoral Immune Response to Recombinant C. trachomatis HtrA

Mice were immunized as described in Example 3. Serum samples from each experimental animal obtained on day 26 were assayed for anti-HtrA IgG levels. Each well of ELISA plates were incubated overnight at 4° C. with 100 μl recombinant C. trachomatis HtrA (2 μg/ml) in 0.1 M NaHCO₃ buffer. Plates were washed four times with 0.01% TWEEN-20™ in PBS then incubated with PBS containing 2% BSA for 2 hours at room temperature. Serum samples were diluted in series in PBS containing 2% BSA. Beginning with an initial dilution of 1:100, samples were diluted ten (10) consecutive times, two to four-fold per dilution, and dispensed into separate wells. Plates were washed four times with 0.01% TWEEN-20™ in PBS then incubated for 1 hour at room temperature with 100 μl horseradish peroxidase conjugated goat anti-mouse IgGγ at 1:10,000 dilution in PBS containing 2% BSA. Plates were washed four times with 0.01% TWEEN-20™ in PBS then incubated for 30 min at room temperature with TMB peroxidase substrate A. TMB stop solution was added and the ELISA plate read at OD₄₅₀. Where OD₄₅₀ values were out of range, repeat dilution series were performed at higher dilutions. ELISA antibody titers of anti-HtrA IgG were expressed as the reciprocal of the serum dilution at the end point of titration, OD₄₅₀ value of 0.10.

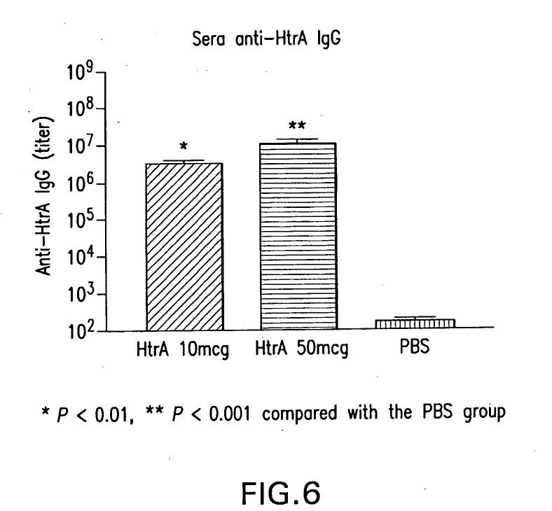

FIG. 6 shows serum anti-HtrA IgG titers after immunization with either 10 or 50 μg recombinant C. trachomatis HtrA or PBS. ELISA plate wells were coated with HtrA then incubated with a dilution series of serum samples. IgG binding was detected by horseradish peroxidase conjugated second antibody binding. Antibody titers are expressed as the reciprocal of serum dilution at the end point of titration. * P<0.01, ** P<0.001 compared with PBS controls.

As shown in FIG. 6, serum anti-HtrA IgG was significantly elevated in animals immunized with either 10 or 50 μg recombinant HtrA, relative to PBS controls. This suggests that C. trachomatis HtrA is strongly immunogenic.

### Example 5

### Immunoprotective Effect of Immunization with Recombinant C. trachomatis HtrA

Mice were immunized as described in Example 3. At day 28, 6 mice from each group were administered a 2.5 mg subcutaneous injection of depo-provera. At day 35, the same 6 mice from each group were vaginally challenged with 3×10⁶ IFU of C. trachomatis serovar E in 20 μl SPG and, also, administered a second 2.5 mg subcutaneous injection of depo-provera. Vaginal samples were collected using swabs at day 3, 7, 10 and 14-post challenge. IFU were determined for each vaginal swab sample, as described in Example 3.

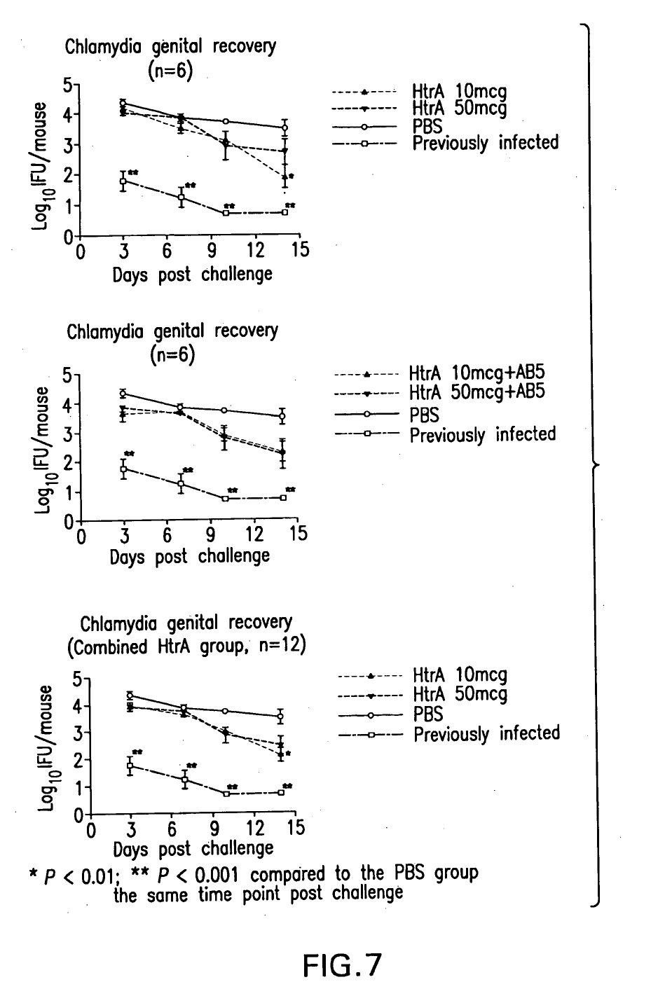

FIG. 7 shows the effect of prior immunization with recombinant C. trachomatis HtrA on the level of genital tract infection after vaginal challenge with serovar E. IFU C. trachomatis in vaginal samples taken at days 3, 7, 10 and 14 after challenge are shown on log scale for animals immunized with 10 or 50 μg recombinant HtrA alone (panel A); with 10 or 50 μg recombinant HtrA and 5 μg mLT (panel B); with PBS or with C. trachomatis serovar E (both panels A and B). * P<0.01; ** P<0.001 compared to PBS controls at the same time point post infection.

As shown in FIG. 7, experimental animals immunized nasally with recombinant C. trachomatis HtrA had less bacterial shedding at day 10 and 14 post infection, compared with PBS controls. Mice immunized with both HtrA and mLT (i.e., AB5), an adjuvant candidate, similarly had less bacterial shedding at day 10 and 14 post infection. Mice recovered from previous infection with C. trachomatis serovar E had nearly complete protection against reinfection, as expected.

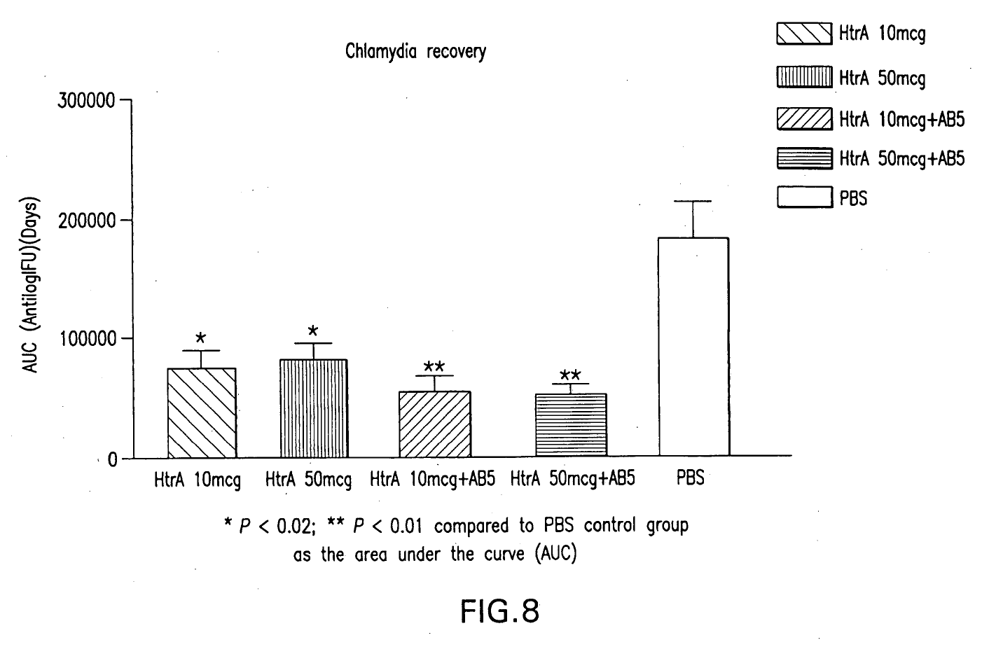

FIG. 8 shows effect of prior immunization with recombinant C. trachomatis HtrA on total vaginal exposure to C. trachomatis during 14 days post-infection of animals subject to vaginal challenge with serovar E. Total vaginal exposure was calculated as the sum across all measurement days of IFU C. trachomatis detected in vaginal samples x days elapsed since last measurement and is shown for PBS controls and for animals immunized with 10 or 50 μg HtrA alone, or with 10 or 50 μg HtrA and 5 μg mLT (i.e., AB5). * P<0.01; ** P<0.001 compared to PBS.

As shown in FIG. 8, when data are expressed as total vaginal exposure, or area under the genital recovery curve (AUC), a slight adjuvant effect is observed. Total exposure (AUC) for each experimental animal was calculated as the sum across all four measurements at days 3, 7, 10 and 14 post-infection of IFU x days elapsed since last measurement. Mice immunized with either 10 or 50 μg recombinant HtrA either with or without mLT (i.e., AB5) had significantly reduced total exposure to infectious C. trachomatis, compared with PBS controls. Mice immunized with both HtrA and mLT had slightly lower total exposure than mice immunized with HtrA alone.

These results demonstrate that recombinant C. trachomatis HtrA induces protective immunity in vivo.

### Example 6

### Inactivation of Inherent C. trachomatis HtrA Protease Activity

To extend the utility of the recombinant HtrA protein to prevent and/or treat Chlamydia sp. infection and disease, the protein's inherent protease activity can be ablated or reduced without adversely affecting the immunogenicity or antigenicity of the protein using a variety of standard genetic and molecular biology methodologies. Protease-deficient HtrA forms would be more amenable for combination with other protein antigens and/or other vaccines containing protein antigens (e.g. the Merck HPV vaccine, Guardisil) to produce a stable multivalent Chlamydia vaccines and/or stable multi-agent vaccines.

Protease-deficient HtrA forms can be produced using conventional site-directed mutagenesis techniques to change one or more of the three serine protease catalytic residues in the protein (e.g. His137, Asp155, Ser247 in SEQ NO: 2) to non-catalytically active residues (e.g. substituting Ala for His at residue position 137; H137A). Other residues within the HtrA protein may also be changed to reduce or eliminate protease activity. Similarly, one or more of the catalytic triad residues or other residues or various segments within the HtrA protein could be deleted to ablate protease activity and generate a highly stable vaccine component.

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

1-59. (canceled)

60. An isolated polynucleotide which encodes a polypeptide comprising an amino acid sequence at least 75% identical to a reference amino acid sequence selected from a group consisting of amino acids 1-19 of SEQ ID NO: 2; amino acids 1-68 of SEQ ID NO: 2, amino acids 1-79 of SEQ ID NO: 2; amino acids 1-112 of SEQ ID NO: 2; amino acids 1-144 of SEQ ID NO: 2; amino acids 1-218 of SEQ ID NO: 2; amino acids 1-299 of SEQ ID NO: 2; amino acids 1-326 of SEQ ID NO: 2; amino acids 1-392 of SEQ ID NO: 2; amino acids 1-436 of SEQ ID NO: 2; amino acids 1-449 of SEQ ID NO: 2; amino acids 17-68 of SEQ ID NO: 2; amino acids 17-79 of SEQ ID NO: 2; amino acids 17-112 of SEQ ID NO: 2; amino acids 17-144 of SEQ ID NO: 2; amino acids 17-218 of SEQ ID NO: 2; amino acids 17-299 of SEQ ID NO: 2; amino acids 17-326 of SEQ ID NO: 2; amino acids 17-392 of SEQ ID NO: 2; amino acids 17-436 of SEQ ID NO: 2; amino acids 17-449 of SEQ ID NO: 2; amino acids 20-68 of SEQ ID NO: 2; amino acids 20-79 of SEQ ID NO: 2; amino acids 20-112 of SEQ ID NO: 2; amino acids 20-144 of SEQ ID NO: 2; amino acids 20-218 of SEQ ID NO: 2; amino acids 20-299 of SEQ ID NO: 2; amino acids 20-326 of SEQ ID NO: 2; amino acids 20-392 of SEQ ID NO: 2; amino acids 20-436 of SEQ ID NO: 2; amino acids 20-449 of SEQ ID NO: 2; amino acids 20-497 of SEQ ID NO: 2; amino acids 69-112 of SEQ ID NO: 2; amino acids 69-144 of SEQ ID NO: 2; amino acids 69-218 of SEQ ID NO: 2; amino acids 69-299 of SEQ ID NO: 2; amino acids 69-326 of SEQ ID NO: 2; amino acids 69-392 of SEQ ID NO: 2; amino acids 69-436 of SEQ ID NO: 2; amino acids 69-449 of SEQ ID NO: 2; amino acids 69-497 of SEQ ID NO: 2; amino acids 80-112 of SEQ ID NO: 2; amino acids 80-144 of SEQ ID NO: 2; amino acids 80-218 of SEQ ID NO: 2; amino acids 80-299 of SEQ ID NO: 2; amino acids 80-326 of SEQ ID NO: 2; amino acids 80-392 of SEQ ID NO: 2; amino acids 80-436 of SEQ ID NO: 2; amino acids 80-449 of SEQ ID NO: 2; amino acids 80-497 of SEQ ID NO: 2; amino acids 113-144 of SEQ ID NO: 2; amino acids 113-218 of SEQ ID NO: 2; amino acids 113-299 of SEQ ID NO: 2; amino acids 113-326 of SEQ ID NO: 2; amino acids 113-392 of SEQ ID NO: 2; amino acids 113-436 of SEQ ID NO: 2; amino acids 113-449 of SEQ ID NO: 2; amino acids 113-497 of SEQ ID NO: 2; amino acids 145-218 of SEQ ID NO: 2; amino acids 145-299 of SEQ ID NO: 2; amino acids 145-326 of SEQ ID NO: 2; amino acids 145-392 of SEQ ID NO: 2; amino acids 145-436 of SEQ ID NO: 2; amino acids 145-449 of SEQ ID NO: 2; amino acids 145-497 of SEQ ID NO: 2; amino acids 219-299 of SEQ ID NO: 2; amino acids 219-326 of SEQ ID NO: 2; amino acids 219-392 of SEQ ID NO: 2; amino acids 219-436 of SEQ ID NO: 2; amino acids 219-449 of SEQ ID NO: 2; amino acids 219-497 of SEQ ID NO: 2; amino acids 300-392 of SEQ ID NO: 2; amino acids 300-436 of SEQ ID NO: 2; amino acids 300-449 of SEQ ID NO: 2; amino acids 300-497 of SEQ ID NO: 2; amino acids 327-392 of SEQ ID NO: 2; amino acids 327-436 of SEQ ID NO: 2; amino acids 327-449 of SEQ ID NO: 2; amino acids 327-497 of SEQ ID NO: 2; amino acids 393-436 of SEQ ID NO: 2; amino acids 393-449 of SEQ ID NO: 2; amino acids 393-497 of SEQ ID NO: 2; amino acids 437-497 of SEQ ID NO: 2; amino acids 450-497 of SEQ ID NO: 2; SEQ ID NO: 30; and a combination of at least two of any of said polypeptide fragments or variants or derivatives thereof,
provided that said amino acid sequence is not SEQ ID NO: 6, or SEQ ID NO: 8,
wherein said polynucleotide or polypeptide when administered to a subject in need thereof in a sufficient amount, induces an immune response against Chlamydia sp.

61. The polynucleotide of claim 60, wherein said polypeptide has one or more amino acid substitutions at a residue selected from the group consisting of:
residue 105 of SEQ ID NO: 2;
residue 137 of SEQ ID NO: 2;
residue 143 of SEQ ID NO: 2;
residue 155 of SEQ ID NO: 2;
residue 247 of SEQ ID NO: 2; and,
a combination of two or more of said residues.

62. The polynucleotide of claim 60, which encodes SEQ ID NO: 2 or amino acids 21-503 of SEQ ID NO: 4.

63. The polynucleotide of claim 60, further comprising a heterologous nucleic acid.

64. The polynucleotide of claim 60, wherein the coding region encoding said polypeptide is codon-optimized.

65. The polynucleotide of claim 64, wherein said coding region is codon-optimized for expression in E. coli, P. fluorescens, or human.

66. A vector comprising the polynucleotide of claim 60.

67. A vector of claim 66, wherein said vector is a vaccinia virus vector.

68. A host cell comprising the vector of claim 66.

69. An isolated polypeptide encoded by the polynucleotide of claim 60.

70. A composition comprising the polynucleotide of claim 60 and a pharmaceutically acceptable carrier.

71. The composition of claim 70, further comprising an adjuvant.

72. A composition comprising the polypeptide of claim 70 and a pharmaceutically acceptable carrier.

73. The composition of claim 72, further comprising an adjuvant.

74. A method of producing a polypeptide, comprising culturing the host cell of claim 68, and recovering said polypeptide.

75. A method of inducing an immune response against Chlamydia in a subject comprising administering to said subject in need thereof an effective amount of the polynucleotide of claim 60.

76. A method of inducing an immune response against Chlamydia in a subject comprising administering to said subject in need thereof an effective amount of the polypeptide of claim 69.

77. A method to treat, prevent, attenuate or ameliorate a Chlamydia infection in a subject comprising administering to said subject in need thereof the polypeptide of claim 69.

78. The method of claim 75, wherein said Chlamydia infection is Chlamydia bacterial infection, trachoma, conjunctivitis, urethritis, lymphogranuloma venereum (LGV), cervicitis, epididymitis, endometritis, pelvic inflammatory disease (PID), salpingitis, tubal occlusion, infertility, cervical cancer, and artherosclerosis.

79. A method to attenuate or ameliorate a symptom caused by a Chlamydia infection in a subject comprising administering to said subject in need thereof the polynucleotide of claim 60.

80. A method to attenuate or ameliorate a symptom caused by a Chlamydia infection in a subject comprising administering to said subject in need thereof the polypeptide of claim 69.

81. A method for determining the presence of nucleic acids of Chlamydia sp. in a test sample, comprising the steps of: a) contacting the test sample with the polynucleotide of claim 60 or complement sequence thereof to produce duplexes; and b) determining the production of duplexes.

82. A method of detecting Chlamydia in a test sample comprising the steps of:
(a) contacting the test sample with the antibody against the polypeptide of claim 69 to form Chlamydia antigen: antibody immunocomplexes, and further, b) detecting the presence of or measuring the amount of said immunocomplexes formed during step a).
